(19) United States
(12) Patent Application Publication
(10) Pub. No.: US 2023/0265105 A1
(43) Pub. Date: Aug. 24, 2023

(54) MACROCYCLIC 7-PYRAZOL-5-YL-INDOLE DERIVATIVES AS INHIBITORS OF MCL-1

(71) Applicant: Janssen Pharmaceutica NV, Beerse (BE)

(72) Inventors: Frederik Jan Rita ROMBOUTS, Wilrijk (BE); Aldo PESCHIULLI, Antwerp (BE); Samuël Dominique DEMIN, Mechelen (BE); Adriana-Ingrid VELTER, Antwerpen (BE); Matthieu Dominique JOUFFROY, Berchem (BE)

(21) Appl. No.: 17/998,957

(22) PCT Filed: May 27, 2021
(86) PCT No.: PCT/EP2021/064162

§ 371 Date: Nov. 16, 2022

(30) Foreign Application Priority Data
May 29, 2020 (EP) 20177468.4
Oct. 26, 2020 (EP) 20203934.3

(51) Int. Cl. C07D 515/22

(52) U.S. Cl. CPC C07D 515/22

(57) ABSTRACT

The present invention relates to pharmaceutical agents useful for therapy and/or prophylaxis in a subject, pharmaceutical composition comprising such compounds, and their use as MCL-1 inhibitors, useful for treating diseases such as cancer.

### FIELD OF THE INVENTION

The present invention relates to pharmaceutical agents useful for therapy and/or prophylaxis in a subject, pharmaceutical composition comprising such compounds, and their use as MCL-1 inhibitors, useful for treating or preventing diseases such as cancer.

### BACKGROUND OF THE INVENTION

Cellular apoptosis or programmed cell death is critical to the development and homeostasis of many organs including the hematopoietic system. Apoptosis can be initiated via the extrinsic pathway, which is mediated by death receptors, or by the intrinsic pathway using the B cell lymphoma (BCL-2) family of proteins. Myeloid cell leukemia-1 (MCL-1) is a member of the BCL-2 family of cell survival regulators and is a critical mediator of the intrinsic apoptosis pathway. MCL-1 is one of five principal anti-apoptotic BCL-2 proteins (MCL-1, BCL-2, BCL-XL, BCL-w, and BFL1/A1) responsible for maintaining cell survival. MCL-1 continuously and directly represses the activity of the pro-apoptotic BCL-2 family proteins Bak and Bax and indirectly blocks apoptosis by sequestering BH3 only apoptotic sensitizer proteins such as Bim and Noxa. The activation of Bak/Bax following various types of cellular stress leads to aggregation on the mitochondrial outer membrane and this aggregation facilitates pore formation, loss of mitochondrial outer membrane potential, and subsequent release of cytochrome C into the cytosol. Cytosolic cytochrome C binds Apaf-1 and initiates recruitment of procaspase 9 to form apoptosome structures (Cheng et al. eLife 2016; 5: e17755). The assembly of apoptosomes activates the executioner cysteine proteases 3/7 and these effector caspases then cleave a variety of cytoplasmic and nuclear proteins to induce cell death (Julian et al. Cell Death and Differentiation 2017; 24, 1380-1389).

Avoiding apoptosis is an established hallmark of cancer development and facilitates the survival of tumor cells that would otherwise be eliminated due to oncogenic stresses, growth factor deprivation, or DNA damage (Hanahan and Weinberg. Cell 2011; 1-44). Thus, unsurprisingly, MCL-1 is highly upregulated in many solid and hematologic cancers relative to normal non-transformed tissue counterparts. The overexpression of MCL-1 has been implicated in the pathogenesis of several cancers where it correlated with poor outcome, relapse, and aggressive disease. Additionally, overexpression of MCL-1 has been implicated in the pathogenesis of the following cancers: prostate, lung, pancreatic, breast, ovarian, cervical, melanoma, B-cell chronic lymphocytic leukemia (CLL), acute myeloid leukemia (AML), and acute lymphoblastic leukemia (ALL). The human MCL-1 genetic locus (1q21) is frequently amplified in tumors and quantitatively increases total MCL-1 protein levels (Beroukhim et al. Nature 2010; 463 (7283) 899-905). MCL-1 also mediates resistance to conventional cancer therapeutics and is transcriptionally upregulated in response to inhibition of BCL-2 function (Yecies et al. Blood 2010; 115 (16)3304-3313).

A small molecule BH3 inhibitor of BCL-2 has demonstrated clinical efficacy in patients with chronic lymphocytic leukemia and is FDA approved for patients with CLL or AML (Roberts et al. NEJM 2016; 374:311-322). The clinical success of BCL-2 antagonism led to the development of several MCL-1 BH3 mimetics that show efficacy in preclinical models of both hematologic malignancies and solid tumors (Kotschy et al. Nature 2016; 538 477-486, Merino et al. Sci. Transl. Med; 2017 (9)).

MCL-1 regulates several cellular processes in addition to its canonical role in mediating cell survival including mitochondrial integrity and non-homologous end joining following DNA damage (Chen et al. JCI 2018; 128(1):500-516). The genetic loss of MCL-1 shows a range of phenotypes depending on the developmental timing and tissue deletion. MCL-1 knockout models reveal there are multiple roles for MCL-1 and loss of function impacts a wide range of phenotypes. Global MCL-1-deficient mice display embryonic lethality and studies using conditional genetic deletion have reported mitochondrial dysfunction, impaired activation of autophagy, reductions in B and T lymphocytes, increased B and T cell apoptosis, and the development of heart failure/cardiomyopathy (Wang et al. Genes and Dev 2013; 27 1351-1364, Steimer et al. Blood 2009(113) 2805-2815).

WO2018178226 discloses MCL-1 inhibitors and methods of use thereof.

WO2017182625 discloses macrocyclic MCL-1 inhibitors for treating cancer.

WO2018178227 discloses the synthesis of MCL-1 inhibitors.

WO2007008627 discloses substituted phenyl derivatives as inhibitors of the activity of anti-apoptotic MCL-1 protein.

WO2008130970 discloses 7-nonsubstituted indole MCL-1 inhibitors.

WO2008131000 discloses 7-substituted indole MCL-1 inhibitors.

WO2020063792 discloses indole macrocyclic derivatives.

WO2020103864 discloses macrocyclic indoles as MCL-1 inhibitors.

WO2020151738 discloses macrocyclic fused pyrrazoles as MCL-1 inhibitors.

WO2020185606 discloses macrocyclic compounds as MCL-1 inhibitors.

There remains a need for MCL-1 inhibitors, useful for the treatment or prevention of cancers such as prostate, lung, pancreatic, breast, ovarian, cervical, melanoma, B-cell chronic lymphocytic leukemia (CLL), acute myeloid leukemia (AML), and acute lymphoblastic leukemia (ALL).

### SUMMARY OF THE INVENTION

The present invention concerns novel compounds of Formula (I)

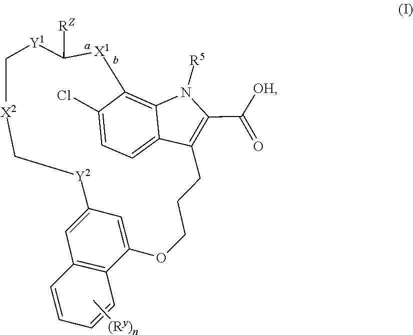

and the tautomers and the stereoisomeric forms thereof, wherein
X¹ represents

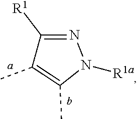

wherein ‘a’ and ‘b’ indicate how variable X¹ is attached to the remainder of the molecule;
R^{y} represents halo;
n represents 0, 1 or 2;
R^{z} means hydrogen or C₁₋₄alkyl;
X² represents

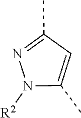

which can be attached to the remainder of the molecule in both directions;
R¹ represents hydrogen; or C₁₋₆alkyl optionally substituted with one substituent selected from the group consisting of Het¹, NR^{6a}R^{6b}, and —OR³;
R² represents hydrogen; methyl; or C₂₋₆alkyl optionally substituted with one substituent selected from the group consisting of Het¹, NR^{6a}R^{6b}, and —OR³;
R^{1a} represents methyl or ethyl;
R³ represents hydrogen, C₁₋₄alkyl, or —C₂₋₄alkyl-O—C₁₋₄alkyl;
R⁵ represents hydrogen; or C₁₋₄alkyl optionally substituted with one substituent selected from the group consisting of C₃₋₆cycloalkyl, Het¹, OR⁴, and NR^{6a}R^{6b};
Het¹ represents morpholinyl or tetrahydropyranyl;
R⁴ represents hydrogen, C₁₋₄alkyl, or —C₂₋₄alkyl-O—C₁₋₄alkyl;
R^{6a} and R^{6b} each independently represent hydrogen or C₁₋₄alkyl;
Y¹ represents —S—, —O—, —CH₂—;
Y² represents —(CH₂)ₘ— or —S—;
m represents 1 or 2;
and the pharmaceutically acceptable salts and the solvates thereof.

The present invention also relates to a pharmaceutical composition comprising a therapeutically effective amount of a compound of Formula (I), a pharmaceutically acceptable salt, or a solvate thereof, and a pharmaceutically acceptable carrier or excipient.

Additionally, the invention relates to a compound of Formula (I), a pharmaceutically acceptable salt, or a solvate thereof, for use as a medicament, and to a compound of Formula (I), a pharmaceutically acceptable salt, or a solvate thereof, for use in the treatment or in the prevention of cancer.

In a particular embodiment, the invention relates to a compound of Formula (I), a pharmaceutically acceptable salt, or a solvate thereof, for use in the treatment or in the prevention of cancer.

The invention also relates to the use of a compound of Formula (I), a pharmaceutically acceptable salt, or a solvate thereof, in combination with an additional pharmaceutical agent for use in the treatment or prevention of cancer.

Furthermore, the invention relates to a process for preparing a pharmaceutical composition according to the invention, characterized in that a pharmaceutically acceptable carrier is intimately mixed with a therapeutically effective amount of a compound of Formula (I), a pharmaceutically acceptable salt, or a solvate thereof.

The invention also relates to a product comprising a compound of Formula (I), a pharmaceutically acceptable salt, or a solvate thereof, and an additional pharmaceutical agent, as a combined preparation for simultaneous, separate or sequential use in the treatment or prevention of cancer.

Additionally, the invention relates to a method of treating or preventing a cell proliferative disease in a subject which comprises administering to the said subject an effective amount of a compound of Formula (I), a pharmaceutically acceptable salt, or a solvate thereof, as defined herein, or a pharmaceutical composition or combination as defined herein.

### DETAILED DESCRIPTION OF THE INVENTION

The term ‘halo’ or ‘halogen’ as used herein represents fluoro, chloro, bromo and iodo.

The prefix ‘C_{x-y}’ (where x and y are integers) as used herein refers to the number of carbon atoms in a given group. Thus, a C₁₋₆alkyl group contains from 1 to 6 carbon atoms, and so on.

The term ‘C₁₋₄alkyl’ as used herein as a group or part of a group represents a straight or branched chain fully saturated hydrocarbon radical having from 1 to 4 carbon atoms, such as methyl, ethyl, n-propyl, isopropyl, n-butyl, s-butyl, t-butyl and the like.

The term ‘C₁₋₆alkyl’ as used herein as a group or part of a group represents a straight or branched chain fully saturated hydrocarbon radical having from 1 to 6 carbon atoms, such as methyl, ethyl, n-propyl, isopropyl, n-butyl, s-butyl, t-butyl, n-pentyl, n-hexyl and the like.

The term ‘C₂₋₄alkyl’ as used herein as a group or part of a group represents a straight or branched chain fully saturated hydrocarbon radical having from 2 to 4 carbon atoms, such as ethyl, n-propyl, isopropyl, n-butyl, s-butyl, t-butyl and the like.

The term ‘C₂₋₆alkyl’ as used herein as a group or part of a group represents a straight or branched chain fully saturated hydrocarbon radical having from 2 to 6 carbon atoms, such as ethyl, n-propyl, isopropyl, n-butyl, s-butyl, t-butyl, n-pentyl, n-hexyl and the like.

The term ‘C₃₋₆cycloalkyl’ as used herein as a group or part of a group defines a fully saturated, cyclic hydrocarbon radical having from 3 to 6 carbon atoms, such as cyclopropyl, cyclobutyl, cyclopentyl and cyclohexyl.

It will be clear for the skilled person that CO or C(═O) represents a carbonyl moiety.

In general, whenever the term ‘substituted’ is used in the present invention, it is meant, unless otherwise indicated or clear from the context, to indicate that one or more hydrogens, in particular from 1 to 4 hydrogens, more in particular from 1 to 3 hydrogens, preferably 1 or 2 hydrogens, more preferably 1 hydrogen, on the atom or radical indicated in the expression using ‘substituted’ are replaced with a selection from the indicated group, provided that the normal valency is not exceeded, and that the substitution results in a chemically stable compound, i.e. a compound that is sufficiently robust to survive isolation to a useful degree of purity from a reaction mixture.

Combinations of substituents and/or variables are permissible only if such combinations result in chemically stable compounds. ‘Stable compound’ is meant to indicate a compound that is sufficiently robust to survive isolation to a useful degree of purity from a reaction mixture.

The skilled person will understand that the term ‘optionally substituted’ means that the atom or radical indicated in the expression using ‘optionally substituted’ may or may not be substituted (this means substituted or unsubstituted respectively).

When two or more substituents are present on a moiety they may, where possible and unless otherwise indicated or clear from the context, replace hydrogens on the same atom or they may replace hydrogen atoms on different atoms in the moiety.

Het¹ may be attached to the remainder of the molecule of Formula (I) through any available ring carbon or nitrogen atom as appropriate, if not otherwise specified.

It will be clear that a Compound of Formula (I) includes Compounds of Formula (I-x) and (I-y) (both directions of X² being

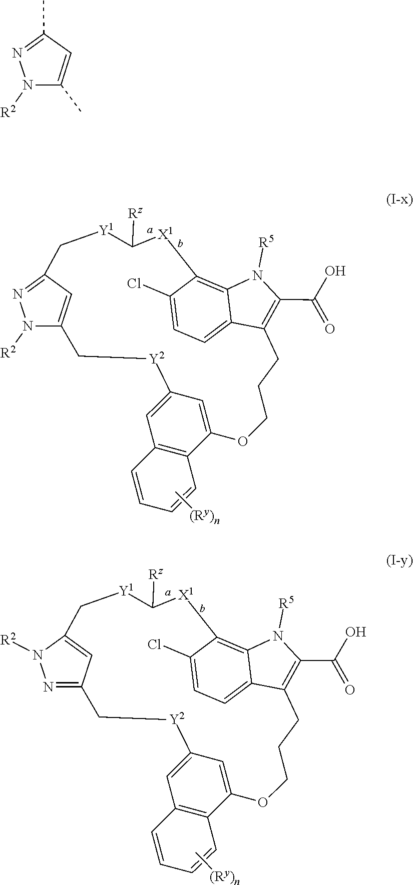

When any variable occurs more than one time in any constituent, each definition is independent.

When any variable occurs more than one time in any formula (e.g. Formula (I)), each definition is independent.

The term “subject” as used herein, refers to an animal, preferably a mammal (e.g. cat, dog, primate or human), more preferably a human, who is or has been the object of treatment, observation or experiment.

The term “therapeutically effective amount” as used herein, means that amount of active compound or pharmaceutical agent that elicits the biological or medicinal response in a tissue system, or subject (e.g., human) that is being sought by a researcher, veterinarian, medicinal doctor or other clinician, which includes alleviation or reversal of the symptoms of the disease or disorder being treated.

The term “composition” is intended to encompass a product comprising the specified ingredients in the specified amounts, as well as any product which results, directly or indirectly, from combinations of the specified ingredients in the specified amounts.

The term “treatment”, as used herein, is intended to refer to all processes wherein there may be a slowing, interrupting, arresting or stopping of the progression of a disease, but does not necessarily indicate a total elimination of all symptoms.

The term “compound(s) of the (present) invention” or “compound(s) according to the (present) invention” as used herein, is meant to include the compounds of Formula (I) and the pharmaceutically acceptable salts, and the solvates thereof.

As used herein, any chemical formula with bonds shown only as solid lines and not as solid wedged or hashed wedged bonds, or otherwise indicated as having a particular configuration (e.g. R, S) around one or more atoms, contemplates each possible stereoisomer, or mixture of two or more stereoisomers.

Hereinbefore and hereinafter, the term “compound(s) of Formula (I)” is meant to include the tautomers thereof and the stereoisomeric forms thereof.

The terms “stereoisomers”, “stereoisomeric forms” or “stereochemically isomeric forms” hereinbefore or hereinafter are used interchangeably.

The invention includes all stereoisomers of the compounds of the invention either as a pure stereoisomer or as a mixture of two or more stereoisomers.

Enantiomers are stereoisomers that are non-superimposable mirror images of each other. A 1:1 mixture of a pair of enantiomers is a racemate or racemic mixture.

Atropisomers (or atropoisomers) are stereoisomers which have a particular spatial configuration, resulting from a restricted rotation about a single bond, due to large steric hindrance. All atropisomeric forms of the compounds of Formula (I) are intended to be included within the scope of the present invention.

In particular, the compounds disclosed herein possess axial chirality, by virtue of restricted rotation around a biaryl bond and as such may exist as mixtures of atropisomers. When a compound is a pure atropisomer, the stereochemistry at each chiral center may be specified by either Rₐ or Sₐ. Such designations may also be used for mixtures that are enriched in one atropisomer. Further description of atropisomerism and axial chirality and rules for assignment of configuration can be found in Eliel, E. L. & Wilen, S. H. ‘Stereochemistry of Organic Compounds’ John Wiley and Sons, Inc. 1994.

Diastereomers (or diastereoisomers) are stereoisomers that are not enantiomers, i.e. they are not related as mirror images. If a compound contains a double bond, the substituents may be in the E or the Z configuration.

Substituents on bivalent cyclic saturated or partially saturated radicals may have either the cis- or trans-configuration; for example if a compound contains a disubstituted cycloalkyl group, the substituents may be in the cis or trans configuration.

Therefore, the invention includes enantiomers, atropisomers, diastereomers, racemates, E isomers, Z isomers, cis isomers, trans isomers and mixtures thereof, whenever chemically possible.

The meaning of all those terms, i.e. enantiomers, atropisomers, diastereomers, racemates, E isomers, Z isomers, cis isomers, trans isomers and mixtures thereof are known to the skilled person.

The absolute configuration is specified according to the Cahn-Ingold-Prelog system. The configuration at an asymmetric atom is specified by either R or S. Resolved stereoisomers whose absolute configuration is not known can be designated by (+) or (−) depending on the direction in which they rotate plane polarized light. For instance, resolved enantiomers whose absolute configuration is not known can be designated by (+) or (−) depending on the direction in which they rotate plane polarized light. Optically active (Rₐ)- and (Sₐ)-atropisomers may be prepared using chiral synthons, chiral reagents or chiral catalysts, or resolved using conventional techniques well known in the art, such as chiral HPLC.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other stereoisomers. Thus, when a compound of Formula (I) is for instance specified as (R), this means that the compound is substantially free of the (S) isomer; when a compound of Formula (I) is for instance specified as E, this means that the compound is substantially free of the Z isomer; when a compound of Formula (I) is for instance specified as cis, this means that the compound is substantially free of the trans isomer; when a compound of Formula (I) is for instance specified as Rₐ, this means that the compound is substantially free of the Sₐ atropisomer.

Pharmaceutically acceptable salts, in particular pharmaceutically acceptable additions salts, include acid addition salts and base addition salts. Such salts may be formed by conventional means, for example by reaction of a free acid or a free base form with one or more equivalents of an appropriate base or acid, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable salts as mentioned hereinabove or hereinafter are meant to comprise the therapeutically active non-toxic acid and base salt forms which the compounds of Formula (I), and solvates thereof, are able to form.

Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds of Formula (I) and solvates thereof containing an acidic proton may also be converted into their non-toxic metal or amine salt forms by treatment with appropriate organic and inorganic bases.

Appropriate base salt forms comprise, for example, the ammonium salts, the alkali and earth alkaline metal salts, e.g. the lithium, sodium, potassium, cesium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The term solvate comprises the solvent addition forms as well as the salts thereof, which the compounds of Formula (I) are able to form. Examples of such solvent addition forms are e.g. hydrates, alcoholates and the like.

The compounds of the invention as prepared in the processes described below may be synthesized in the form of mixtures of enantiomers, in particular racemic mixtures of enantiomers, that can be separated from one another following art-known resolution procedures. A manner of separating the enantiomeric forms of the compounds of Formula (I), and pharmaceutically acceptable salts, and solvates thereof, involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

The term “enantiomerically pure” as used herein means that the product contains at least 80% by weight of one enantiomer and 20% by weight or less of the other enantiomer. Preferably the product contains at least 90% by weight of one enantiomer and 10% by weight or less of the other enantiomer. In the most preferred embodiment the term “enantiomerically pure” means that the composition contains at least 99% by weight of one enantiomer and 1% or less of the other enantiomer.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature).

All isotopes and isotopic mixtures of any particular atom or element as specified herein are contemplated within the scope of the compounds of the invention, either naturally occurring or synthetically produced, either with natural abundance or in an isotopically enriched form. Exemplary isotopes that can be incorporated into compounds of the invention include isotopes of hydrogen, carbon, nitrogen, oxygen, phosphorus, sulfur, fluorine, chlorine and iodine, such as ², ³H, ¹¹C, ¹³C, ¹⁴C, ¹³N, ¹⁵O, ¹⁷O, ¹⁸O, ³²P, ³³P, ³⁵S, ¹⁸F, ³⁶Cl, ¹²²I, ¹²³I, ¹²⁵I, ¹³¹I, ⁷⁵Br, ⁷⁶Br, ⁷⁷Br and ⁸²Br. Preferably, the isotope is selected from the group of ²H, ³H, ¹¹C and ¹⁸F. More preferably, the isotope is ²H. In particular, deuterated compounds are intended to be included within the scope of the present invention.

Certain isotopically-labeled compounds of the present invention (e.g., those labeled with ³H and ¹⁴C) may be useful for example in substrate tissue distribution assays. Tritiated (³H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies. PET imaging in cancer finds utility in helping locate and identify tumours, stage the disease and determine suitable treatment.

Human cancer cells overexpress many receptors or proteins that are potential disease-specific molecular targets. Radiolabelled tracers that bind with high affinity and specificity to such receptors or proteins on tumour cells have great potential for diagnostic imaging and targeted radionuclide therapy (Charron, Carlie L. et al. Tetrahedron Lett. 2016, 57(37), 4119-4127). Additionally, target-specific PET radiotracers may be used as biomarkers to examine and evaluate pathology, by for example, measuring target expression and treatment response (Austin R. et al. Cancer Letters (2016), doi: 10.1016/j.canlet.2016.05.008).

The present invention relates in particular to compounds of Formula (I) as defined herein, and the tautomers and the stereoisomeric forms thereof, wherein

X¹ represents

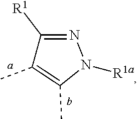

wherein ‘a’ and ‘b’ indicate how variable X¹ is attached to the remainder of the molecule;
R^{y} represents halo;
n represents 0, 1 or 2;
R^{z} means hydrogen or C₁₋₄alkyl;
X² represents

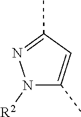

which can be attached to the remainder of the molecule in both directions;
R¹ represents hydrogen; or C₁₋₆alkyl optionally substituted with one substituent selected from the group consisting of Het¹, and —OR³;
R² represents hydrogen; methyl; or C₂₋₆alkyl optionally substituted with one substituent selected from the group consisting of Het¹, and —OR³;
R^{1a} represents methyl or ethyl;
R³ represents hydrogen, C₁₋₄alkyl, or —C₂₋₄alkyl-O—C₁₋₄alkyl;
R⁵ represents hydrogen; or C₁₋₄alkyl optionally substituted with one substituent selected from the group consisting of C₃₋₆cycloalkyl, Het¹, OR⁴, and NR^{6a}R^{6b};
Het¹ represents morpholinyl or tetrahydropyranyl;
R⁴ represents hydrogen, C₁₋₄alkyl, or —C₂₋₄alkyl-O—C₁₋₄alkyl;
R^{6a} and R^{6b} each independently represent hydrogen or C₁₋₄alkyl;
Y¹ represents —S—, —O—, —CH₂—;
Y² represents —(CH₂)ₘ— or —S—;
m represents 1 or 2;
and the pharmaceutically acceptable salts and the solvates thereof.

The present invention relates in particular to compounds of Formula (I) as defined herein, and the tautomers and the stereoisomeric forms thereof, wherein

X¹ represents

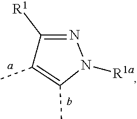

wherein ‘a’ and ‘b’ indicate how variable X¹ is attached to the remainder of the molecule;
R^{y} represents halo;
n represents 0, 1 or 2;
R^{z} means hydrogen or C₁₋₄alkyl;
X² represents

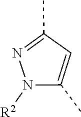

which can be attached to the remainder of the molecule in both directions;
R¹ represents hydrogen; or C₁₋₆alkyl optionally substituted with one substituent selected from the group consisting of Het¹ and —OR³;
R² represents hydrogen; methyl; or C₂₋₆alkyl optionally substituted with one substituent selected from the group consisting of Het¹ and —OR³;
R^{1a} represents methyl or ethyl;
R³ represents hydrogen, C₁₋₄alkyl, or —C₂₋₄alkyl-O—C₁₋₄alkyl;
R⁵ represents C₁₋₄alkyl optionally substituted with one substituent selected from the group consisting of C₃₋₆cycloalkyl and Het¹;
Het¹ represents morpholinyl or tetrahydropyranyl;
Y¹ represents —S—, —O—, —CH₂—;
Y² represents —(CH₂)ₘ— or —S—;
m represents 1 or 2;
and the pharmaceutically acceptable salts and the solvates thereof.

The present invention relates in particular to compounds of Formula (I) as defined herein, and the tautomers and the stereoisomeric forms thereof, wherein

X¹ represents

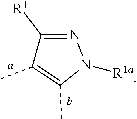

wherein ‘a’ and ‘b’ indicate how variable X¹ is attached to the remainder of the molecule;
R^{y} represents halo;
n represents 0, 1 or 2;
R^{z} means hydrogen;
X² represents

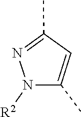

which can be attached to the remainder of the molecule in both directions;
R¹ represents hydrogen; or C₁₋₆alkyl optionally substituted with one —OR³;
R² represents methyl;
R^{1a} represents methyl;
R³ represents —C₂₋₄alkyl-O—C₁₋₄alkyl;
R⁵ represents hydrogen; or C₁₋₄alkyl optionally substituted with one substituent selected from the group consisting of Het¹, OR⁴, and NR^{6a}R^{6b};
Het¹ represents morpholinyl;
R⁴ represents C₁₋₄alkyl or —C₂₋₄alkyl-O—C₁₋₄alkyl;
R^{6a} and R^{6b} represent C₁₋₄alkyl;
Y¹ represents —S—;
Y² represents —(CH₂)ₘ— or —S—;
m represents 1;
and the pharmaceutically acceptable salts and the solvates thereof.

The present invention relates in particular to compounds of Formula (I) as defined herein, and the tautomers and the stereoisomeric forms thereof, wherein

X¹ represents

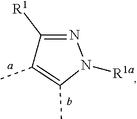

wherein ‘a’ and ‘b’ indicate how variable X¹ is attached to the remainder of the molecule;
R^{y} represents halo;
n represents 0, 1 or 2;
R^{z} means hydrogen;
X² represents

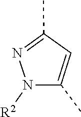

which can be attached to the remainder of the molecule in both directions;
R¹ represents hydrogen; or C₁₋₆alkyl optionally substituted with one —OR³;
R² represents methyl;
R^{1a} represents methyl;
R³ represents —C₂₋₄alkyl-O—C₁₋₄alkyl;
R⁵ represents C₁₋₄alkyl optionally substituted with one Het¹ substituent;
Het¹ represents morpholinyl;
Y¹ represents —S—;
Y² represents —(CH₂)ₘ— or —S—;
m represents 1;
and the pharmaceutically acceptable salts and the solvates thereof.

The present invention relates in particular to compounds of Formula (I) as defined herein, and the tautomers and the stereoisomeric forms thereof, wherein

X¹ represents

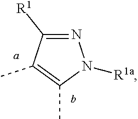

wherein ‘a’ and ‘b’ indicate how variable X¹ is attached to the remainder of the molecule;
R^{y} represents halo;
n represents 0 or 1;
R^{z} means hydrogen;
X² represents

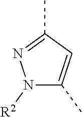

which can be attached to the remainder of the molecule in both directions;
R¹ represents hydrogen; or C₁₋₆alkyl optionally substituted with one —OR³;
R² represents methyl;
R^{1a} represents methyl;
R³ represents —C₂₋₄alkyl-O—C₁₋₄alkyl;
R⁵ represents C₁₋₄alkyl;
Y¹ represents —S—;
Y² represents —(CH₂)ₘ— or —S—;
m represents 1;
and the pharmaceutically acceptable salts and the solvates thereof.

In an embodiment, the present invention relates to those compounds of Formula (I) and the pharmaceutically acceptable salts, and the solvates thereof, or any subgroup thereof as mentioned in any of the other embodiments, wherein

Y¹ represents —S—.

In an embodiment, the present invention relates to those compounds of Formula (I) and the pharmaceutically acceptable salts, and the solvates thereof, or any subgroup thereof as mentioned in any of the other embodiments, wherein R^{y} represents fluoro.

In an embodiment, the present invention relates to those compounds of Formula (I) and the pharmaceutically acceptable salts, and the solvates thereof, or any subgroup thereof as mentioned in any of the other embodiments, wherein

n represents 1; and
R^{y} represents fluoro.

In an embodiment, the present invention relates to those compounds of Formula (I) and the pharmaceutically acceptable salts, and the solvates thereof, or any subgroup thereof as mentioned in any of the other embodiments, wherein

n represents 2; and
R^{y} represents fluoro.

In an embodiment, the present invention relates to those compounds of Formula (I) and the pharmaceutically acceptable salts, and the solvates thereof, or any subgroup thereof as mentioned in any of the other embodiments, wherein

n represents 1 or 2; and
R^{y} represents fluoro.

In an embodiment, the present invention relates to those compounds of Formula (I) and the pharmaceutically acceptable salts, and the solvates thereof, or any subgroup thereof as mentioned in any of the other embodiments, wherein R¹ represents hydrogen.

In an embodiment, the present invention relates to those compounds of Formula (I) and the pharmaceutically acceptable salts, and the solvates thereof, or any subgroup thereof as mentioned in any of the other embodiments, wherein R¹ represents methyl.

In an embodiment, the present invention relates to those compounds of Formula (I) and the pharmaceutically acceptable salts, and the solvates thereof, or any subgroup thereof as mentioned in any of the other embodiments, wherein R² represents methyl.

In an embodiment, the present invention relates to those compounds of Formula (I) and the pharmaceutically acceptable salts, and the solvates thereof, or any subgroup thereof as mentioned in any of the other embodiments, wherein

R¹ represents hydrogen; or
C₂₋₆alkyl optionally substituted with one substituent selected from the group consisting of Het¹ and —OR³.

In an embodiment, the present invention relates to those compounds of Formula (I) and the pharmaceutically acceptable salts, and the solvates thereof, or any subgroup thereof as mentioned in any of the other embodiments, wherein

R¹ represents hydrogen; or
C₂₋₆alkyl optionally substituted with one substituent selected from the group consisting of Het¹ and —OR³; and
R² represents methyl.

In an embodiment, the present invention relates to those compounds of Formula (I) and the pharmaceutically acceptable salts, and the solvates thereof, or any subgroup thereof as mentioned in any of the other embodiments, wherein

R¹ represents C₁₋₆alkyl substituted with one substituent selected from the group consisting of Het¹ and —OR³.

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl substituted with one substituent selected from the group consisting of Het¹ and —OR³.

In an embodiment, the present invention relates to those compounds of Formula (I) and the pharmaceutically acceptable salts, and the solvates thereof, or any subgroup thereof as mentioned in any of the other embodiments, wherein

R¹ represents C₁₋₆alkyl substituted with one substituent selected from the group consisting of Het¹ and —OR³; and
Y² represents —(CH₂)ₘ—.

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl substituted with one substituent selected from the group consisting of Het¹ and —OR³; and
Y² represents —(CH₂)ₘ—.

In an embodiment, the present invention relates to those compounds of Formula (I) and the pharmaceutically acceptable salts, and the solvates thereof, or any subgroup thereof as mentioned in any of the other embodiments, wherein

R¹ is other than methyl.

In an embodiment, the present invention relates to those compounds of Formula (I) and the pharmaceutically acceptable salts, and the solvates thereof, or any subgroup thereof as mentioned in any of the other embodiments, wherein

R¹ represents hydrogen; or
C₁₋₆alkyl substituted with one substituent selected from the group consisting of Het¹ and —OR³; and
R² represents methyl.

In an embodiment, the present invention relates to those compounds of Formula (I) and the pharmaceutically acceptable salts, and the solvates thereof, or any subgroup thereof as mentioned in any of the other embodiments, wherein

R¹ represents hydrogen; or
C₁₋₆alkyl substituted with one —OR³; and
R² represents methyl.

In an embodiment, the present invention relates to those compounds of Formula (I) and the pharmaceutically acceptable salts, and the solvates thereof, or any subgroup thereof as mentioned in any of the other embodiments, wherein

R¹ represents C₁₋₆alkyl substituted with one —OR³; and
R² represents methyl.

In an embodiment, the present invention relates to those compounds of Formula (I) and the pharmaceutically acceptable salts, and the solvates thereof, or any subgroup thereof as mentioned in any of the other embodiments, wherein

R¹ represents methylene substituted with one —OR³; and
R² represents methyl.

In an embodiment, the present invention relates to those compounds of Formula (I) and the pharmaceutically acceptable salts, and the solvates thereof, or any subgroup thereof as mentioned in any of the other embodiments, wherein

R¹ represents methylene substituted with one —OR³;
R² represents methyl; and
R³ represents —C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl optionally substituted with one substituent selected from the group consisting of Het¹ and —OR³; and
R² represents methyl.

In an embodiment, the present invention relates to those compounds of Formula (I) and the pharmaceutically acceptable salts, and the solvates thereof, or any subgroup thereof as mentioned in any of the other embodiments, wherein R⁵ represents methyl.

In an embodiment, the present invention relates to those compounds of Formula (I) and the pharmaceutically acceptable salts, and the solvates thereof, or any subgroup thereof as mentioned in any of the other embodiments, wherein n represents 0.

In an embodiment, the present invention relates to those compounds of Formula (I) and the pharmaceutically acceptable salts, and the solvates thereof, or any subgroup thereof as mentioned in any of the other embodiments, wherein n represents 1.

In an embodiment, the present invention relates to those compounds of Formula (I) and the pharmaceutically acceptable salts, and the solvates thereof, or any subgroup thereof as mentioned in any of the other embodiments, wherein n represents 2.

In an embodiment, the present invention relates to those compounds of Formula (I) and the pharmaceutically acceptable salts, and the solvates thereof, or any subgroup thereof as mentioned in any of the other embodiments, wherein m represents 1.

In an embodiment, the present invention relates to those compounds of Formula (I) and the pharmaceutically acceptable salts, and the solvates thereof, or any subgroup thereof as mentioned in any of the other embodiments, wherein m represents 2.

In an embodiment, the present invention relates to those compounds of Formula (I) and the pharmaceutically acceptable salts, and the solvates thereof, or any subgroup thereof as mentioned in any of the other embodiments, wherein R^{z} represents hydrogen.

In an embodiment, the present invention relates to those compounds of Formula (I) and the pharmaceutically acceptable salts, and the solvates thereof, or any subgroup thereof as mentioned in any of the other embodiments, wherein Y¹ represents —S—.

In an embodiment, the present invention relates to those compounds of Formula (I) and the pharmaceutically acceptable salts, and the solvates thereof, or any subgroup thereof as mentioned in any of the other embodiments, wherein Y² represents —S—.

In an embodiment, the present invention relates to those compounds of Formula (I) and the pharmaceutically acceptable salts, and the solvates thereof, or any subgroup thereof as mentioned in any of the other embodiments, wherein Y² represents —(CH₂)ₘ—.

In an embodiment, the present invention relates to those compounds of Formula (I) and the pharmaceutically acceptable salts, and the solvates thereof, or any subgroup thereof as mentioned in any of the other embodiments, wherein

R³ represents —C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

R¹ represents C₁₋₆alkyl substituted with one —OR³; and
R³ represents —C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

R¹ represents C₁₋₆alkyl substituted with one —OR³;
R³ represents —C₂₋₄alkyl-O—C₁₋₄alkyl; and
n represents 1.

In an embodiment, the present invention relates to those compounds of Formula (I) and the pharmaceutically acceptable salts, and the solvates thereof, or any subgroup thereof as mentioned in any of the other embodiments, wherein

R¹ represents C₁₋₆alkyl substituted with one —OR³;
R³ represents —C₂₋₄alkyl-O—C₁₋₄alkyl;
n represents 1; and
R^{y} represents fluoro.

In an embodiment, the present invention relates to those compounds of Formula (I) and the pharmaceutically acceptable salts, and the solvates thereof, or any subgroup thereof as mentioned in any of the other embodiments, wherein at least one of R¹ and R² is other than unsubstituted methyl.

In an embodiment, the present invention relates to those compounds of Formula (I) and the pharmaceutically acceptable salts, and the solvates thereof, or any subgroup thereof as mentioned in any of the other embodiments, wherein R⁵ represents C₁₋₄alkyl optionally substituted with one substituent selected from the group consisting of C₃₋₆cycloalkyl, Het¹, OR⁴, and NR^{6a}R^{6b}.

In an embodiment, the present invention relates to those compounds of Formula (I) and the pharmaceutically acceptable salts, and the solvates thereof, or any subgroup thereof as mentioned in any of the other embodiments, wherein R⁵ represents C₁₋₄alkyl optionally substituted with one substituent selected from the group consisting of Het¹, OR⁴, and NR^{6a}R^{6b}.

In an embodiment, the present invention relates to those compounds of Formula (I) and the pharmaceutically acceptable salts, and the solvates thereof, or any subgroup thereof as mentioned in any of the other embodiments, wherein R⁵ represents C₁₋₄alkyl substituted with one substituent selected from the group consisting of Het¹, OR⁴, and NR^{6a}R^{6b}.

In an embodiment, the present invention relates to those compounds of Formula (I) and the pharmaceutically acceptable salts, and the solvates thereof, or any subgroup thereof as mentioned in any of the other embodiments, wherein R⁵ represents C₁₋₄alkyl substituted with one substituent selected from the group consisting of C₃₋₆cycloalkyl, Het¹, OR⁴, and NR^{6a}R^{6b}.

In an embodiment, the present invention relates to those compounds of Formula (I) and the pharmaceutically acceptable salts, and the solvates thereof, or any subgroup thereof as mentioned in any of the other embodiments, wherein Het¹ is attached to the remainder of the molecule of Formula (I) through a nitrogen atom.

In an embodiment, the present invention relates to those compounds of Formula (I) and the pharmaceutically acceptable salts, and the solvates thereof, or any subgroup thereof as mentioned in any of the other embodiments, wherein n is 1 and wherein R^{y} is in position 3 as indicated below:

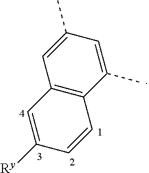

In an embodiment, the present invention relates to those compounds of Formula (I) and the pharmaceutically acceptable salts, and the solvates thereof, or any subgroup thereof as mentioned in any of the other embodiments, wherein n is 1 and wherein R^{y} is in position 3 as indicated below; and wherein R^{y} represents fluoro:

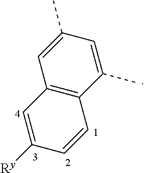

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-x):

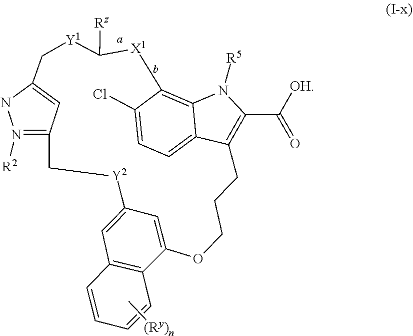

It will be clear that all variables in the structure of Formula (I-x), are defined as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-y):

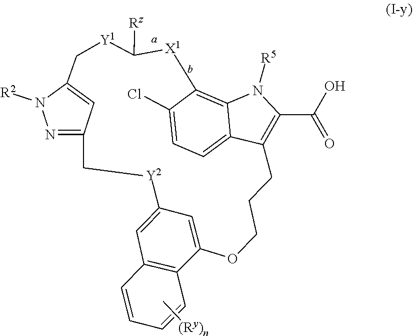

In an embodiment, the present invention relates to those compounds of Formula (I) and the pharmaceutically acceptable salts, and the solvates thereof, or any subgroup thereof as mentioned in any of the other embodiments, provided that

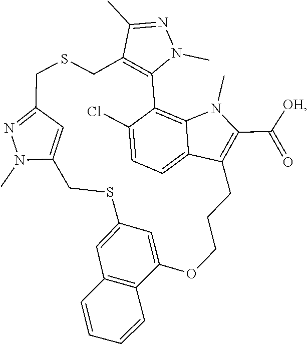 [SMILES: Cc1nn(C)c2c1CSCc1cc(n(C)n1)CSc1cc(c3ccccc3c1)OCCCc1c(C(=O)O)n(C)c3c-2c(Cl)ccc13]

and the tautomers and the stereoisomeric forms thereof are excluded. In an embodiment, the scope of the present invention does not include said excluded compound, and the pharmaceutically acceptable salts thereof. In an embodiment, the scope of the present invention does not include said excluded compound, and the pharmaceutically acceptable salts and the solvates thereof.

It will be clear that all variables in the structure of Formula (I-y), are defined as defined for the compounds of Formula (I) or any subgroup thereof as mentioned in any of the other embodiments.

In an embodiment, the present invention relates to a subgroup of Formula (I) as defined in the general reaction schemes.

In an embodiment the compound of Formula (I) is selected from the group consisting of any of the exemplified compounds, tautomers and stereoisomeric forms thereof, any pharmaceutically acceptable salts, and the solvates thereof.

All possible combinations of the above indicated embodiments are considered to be embraced within the scope of the invention.

### Methods for the Preparation of Compounds

In this section, as in all other sections unless the context indicates otherwise, references to Formula (I) also include all other sub-groups and examples thereof as defined herein.

The general preparation of some typical examples of the compounds of Formula (I) is described hereunder and in the specific examples, and are generally prepared from starting materials which are either commercially available or prepared by standard synthetic processes commonly used by those skilled in the art of organic chemistry. The following schemes are only meant to represent examples of the invention and are in no way meant to be a limit of the invention.

Alternatively, compounds of the present invention may also be prepared by analogous reaction protocols as described in the general schemes below, combined with standard synthetic processes commonly used by those skilled in the art.

The skilled person will realize that in the reactions described in the Schemes, although this is not always explicitly shown, it may be necessary to protect reactive functional groups (for example hydroxy, amino, or carboxy groups) where these are desired in the final product, to avoid their unwanted participation in the reactions. In general, conventional protecting groups can be used in accordance with standard practice. The protecting groups may be removed at a convenient subsequent stage using methods known from the art.

The skilled person will realize that in the reactions described in the Schemes, it may be advisable or necessary to perform the reaction under an inert atmosphere, such as for example under N₂-gas atmosphere.

It will be apparent for the skilled person that it may be necessary to cool the reaction mixture before reaction work-up (refers to the series of manipulations required to isolate and purify the product(s) of a chemical reaction such as for example quenching, column chromatography, extraction).

The skilled person will realize that heating the reaction mixture under stirring may enhance the reaction outcome. In some reactions microwave heating may be used instead of conventional heating to shorten the overall reaction time.

The skilled person will realize that another sequence of the chemical reactions shown in the Schemes below, may also result in the desired compound of Formula (I).

The skilled person will realize that intermediates and final compounds shown in the Schemes below may be further functionalized according to methods well-known by the person skilled in the art. The intermediates and compounds described herein can be isolated in free form or as a salt, or a solvate thereof. The intermediates and compounds described herein may be synthesized in the form of mixtures of tautomers and stereoisomeric forms that can be separated from one another following art-known resolution procedures.

Compounds of Formula (I-x) can be prepared according to Scheme 1,

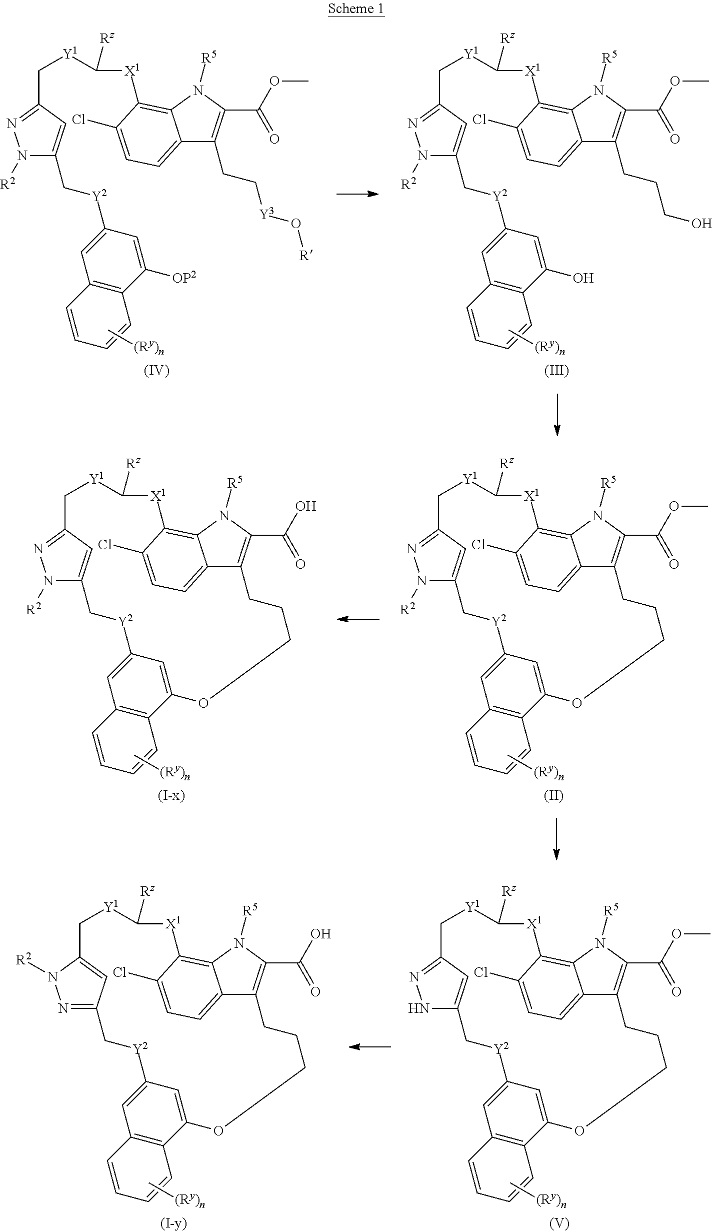

by reacting an intermediate of Formula (II) where X¹, Y¹, Y², R², R⁵, R^{z} and (R^{y})ₙ are defined as in Formula (I), with a suitable base such as, for example, LiOH or NaOH, in a suitable solvent such as water or a mixture of water and a suitable organic solvent such as dioxane or THF, or a mixture of MeOH and THF, at a suitable temperature such as room temperature or 60° C.
An intermediate of Formula (II) might have a protecting group in the R² position such as, for example, tetrahydropyranyl. In such a case, the intermediate of Formula (II) is reacted with a suitable deprotection reagent, such as, for example, pTsOH (p-toluenesulfonic acid) or HCl, in a suitable solvent such as, for example, iPrOH (2-propanol), at a suitable temperature such as, for example, room temperature. In a next step the obtained unprotected intermediate (V) can be reacted with a suitable alkylating agent R²L where L is a suitable leaving group such as, for example, an alkyl halide, in the presence of a suitable base such as, for example, Cs₂CO₃, in a suitable solvent such as, for example, DMF (N,N-dimethylformamide), at a suitable temperature such as, for example, room temperature or 60° C. providing a compound of formula I-y.
Intermediates of Formula (II) can be prepared by reacting an intermediate of Formula (III) wherein X¹, Y¹, Y², R⁵, R^{z}, and (R^{y})ₙ are defined as in Formula (I), and R² is a suitable protecting group such as, for example, tetrahydropyranyl (THP), or can also be a suitable alkyl substituent such as, for example, methyl, with a suitable reagent, such as, for example, diethyl azodicarboxylate (DEAD) or di-tert-butyl azodicarboxylate (DTBAD), in the presence of a suitable phosphine such as, for example, PPh₃, in a suitable solvent such as, for example, THF, toluene, or a mixture thereof, at a suitable temperature such as, for example, room temperature or 70° C.
Intermediates of Formula (III) can be prepared by reacting an intermediate of Formula (IV) wherein X¹, Y¹, Y², R⁵, R^{z}, and (R^{y})ₙ are as defined in Formula (I), Y³ is CH₂, and R′ as well as P² are suitable protecting groups, such as, for example, tert-butyldimethylsilyl (TBDMS) or tert-butyldiphenylsilyl (TBDPS), with a suitable deprotecting reagent such as, for example, tetrabutylammonium fluoride (TBAF), in a suitable solvent such as, for example, THF, at a suitable temperature such as, for example, room temperature or 60° C. It will be clear for somebody skilled in the art, that in case R² is a protective group, R′ and P² will have to be orthogonal protective groups to R².
Alternatively, when P² in intermediates of Formula (IV) is a 4-methoxybenzyl (PMB) group, an additional deprotection step might be necessary, using a suitable deprotection reagent such as, for example, trifluoroacetic acid (TFA) or 2,3-dichloro-5,6-dicyano-1,4-benzoquinone (DDQ), in a suitable solvent such as, for example, DCM, at a suitable temperature such as, for example, room temperature.
Alternatively, when Y³ is C═O and R′ is Me in intermediates of Formula (IV), an additional reduction step might be necessary with a suitable reducing agent such as, for example, BH₃·DMS (borane dimethylsulfide), in a suitable solvent such as, for example, THF, at a suitable temperature such as, for example, room temperature or 50° C.

It will be clear for a skilled person that a compound of formula (I-y) can also be prepared in a similar way as (I-x), starting from intermediates of Formula (II), (III) and (IV) having R² at the isomeric pyrazole position.

It will also be clear for a skilled person that in case R⁵ is H on an intermediate of Formula (II), (IV) or (V), an additional alkylation step can be performed with a suitable alkyl halide R⁵L where L is a suitable leaving group such as, for example, an alkyl halide, in the presence of a suitable base such as, for example, Cs₂CO₃, in a suitable solvent such as, for example, DMF (N,N-dimethylformamide), at a suitable temperature such as, for example, room temperature or 60° C.

Alternatively, intermediates of Formula (II), wherein R⁵ is as defined in Formula I, can be prepared in two steps from an intermediate of Formula (II), wherein R⁵ is a suitable protecting group:
First, by reacting an intermediate of Formula (II), wherein R⁵ is a suitable protecting group such as, for example, SEM (trimethylsilylethoxymethyl), with a suitable deprotecting agent such as, for example, TFA, in a suitable solvent such as, for example, DCM, at a suitable temperature such as, for example, room temperature.
And, in a second step, by reacting the obtained Intermediates of Formula (II), wherein R⁵ is hydrogen, with a suitable alkylating agent such as, for example, a substituted alkyl halide, in the presence of a suitable base such as, for example, Cs₂CO₃, in a suitable solvent such as, for example, DMF, at a suitable temperature such as, for example, 80° C.

Intermediates of Formula (II), wherein R⁵ is a suitable protecting group, can be prepared according to Schemes 1-5, from intermediates of Formula (XIV), wherein R⁵ is a suitable protecting group such as SEM (trimethylsilylethoxymethyl).

Alternatively, Intermediates of Formula (I), wherein R⁵ is an alkoxyalkyl group, can be prepared by reacting an intermediate of Formula (I), wherein R⁵ is an hydroxyalkyl group, with a suitable alkylating agent such as, for example, methyl iodide, in the presence of a suitable base such as, for example, NaH, in a suitable solvent such as, for example, THF, at a suitable temperature such as, for example, 0° C. or room temperature. Intermediates of Formula (IV) wherein X¹, Y², R², R⁵, R^{z}, and (R^{y})ₙ are defined as in Formula (I), Y¹ is S and Y³/R′ is C═O/Me or Y³/R′ is CH₂/TBDMS, can be prepared according to Scheme 2

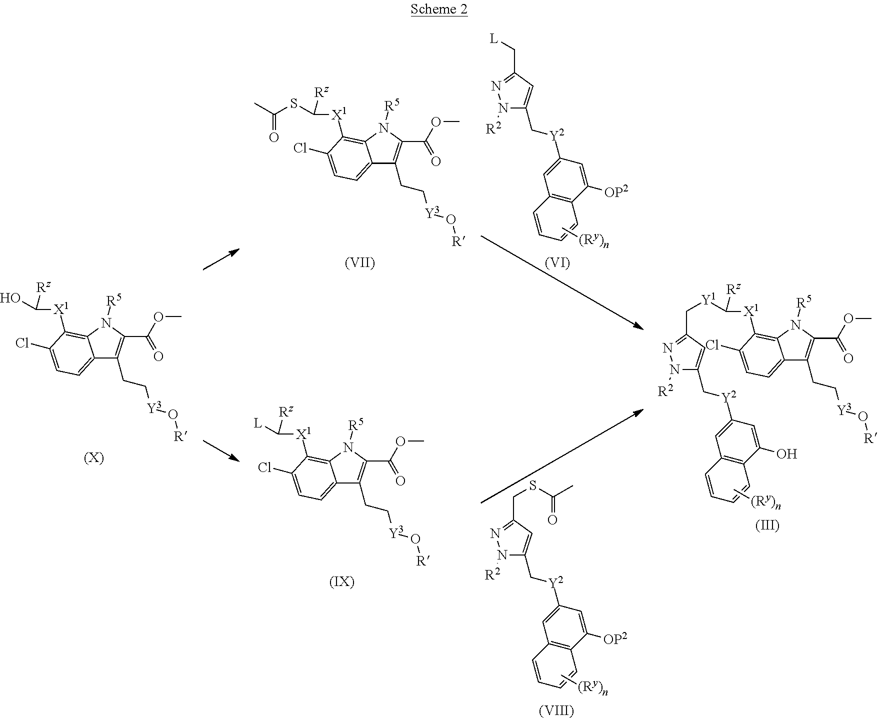

by reacting an intermediate of Formula (VII), where X¹, R^{z} and R⁵ are defined as in Formula (I) with an intermediate of Formula (VI), where R², Y² and (R^{y})ₙ are defined as in Formula (I), and L is defined as a suitable leaving group such as, for example, I, Br, Cl or OMs (methanesulfonate), in the presence of a suitable base such as, for example, K₂CO₃, in a suitable solvent such as, for example, MeOH, THF, or a mixture thereof, at a suitable temperature such as, for example, room temperature.
Alternatively, intermediates of Formula (IV) can be prepared by reacting an intermediate of Formula (IX) where X¹, R^{z} and R⁵ are defined as in Formula (I), and L is defined as a suitable leaving group such as for example I, Br, Cl or OMs, with an intermediate of Formula (VIII), where R², Y² and (R^{y})ₙ are defined as in Formula (I), in the presence of a suitable base such as, for example, K₂CO₃, in a suitable solvent such as, for example, MeOH, THF, or a mixture thereof, at a suitable temperature such as, for example, room temperature.
Intermediates of Formula (VII) can be prepared by reacting an intermediate of Formula (X), in a two-step procedure, first in the presence of a suitable activating agent such as, for example, methanesulfonyl chloride (MsCl), in the presence of a suitable base such as, for example, triethylamine (Et₃N), in a suitable solvent such as, for example, tetrahydrofuran (THF), at a suitable temperature such as, for example, room temperature, then by reacting with potassium thioacetate (AcSK) in a suitable solvent such as, for example, DMF, at a suitable temperature such as, for example, room temperature.
Intermediates of Formula (IX) can be prepared by reacting intermediates of Formula (X) with a suitable activating agent such as, for example, MsCl or SOCl₂, or a mixture of CBr₄ and triphenylphosphine (PPh₃), in a suitable solvent such as DCM, at a suitable temperature such as, for example, room temperature.

It will be clear for somebody skilled in the art, that compounds of Formula (IV) in which R^{z} is hydrogen and Y¹ is S can also be prepared using a similar procedure starting from an intermediate of Formula (XIII).

Intermediates of Formula (IV) wherein X¹, Y², R², R⁵, R^{z}, and (R^{y})ₙ are defined as in Formula (I), Y¹ is O and Y³/R′ is C═O/Me or Y³/R′ is CH₂/TBDMS, can be prepared according to Scheme 3,

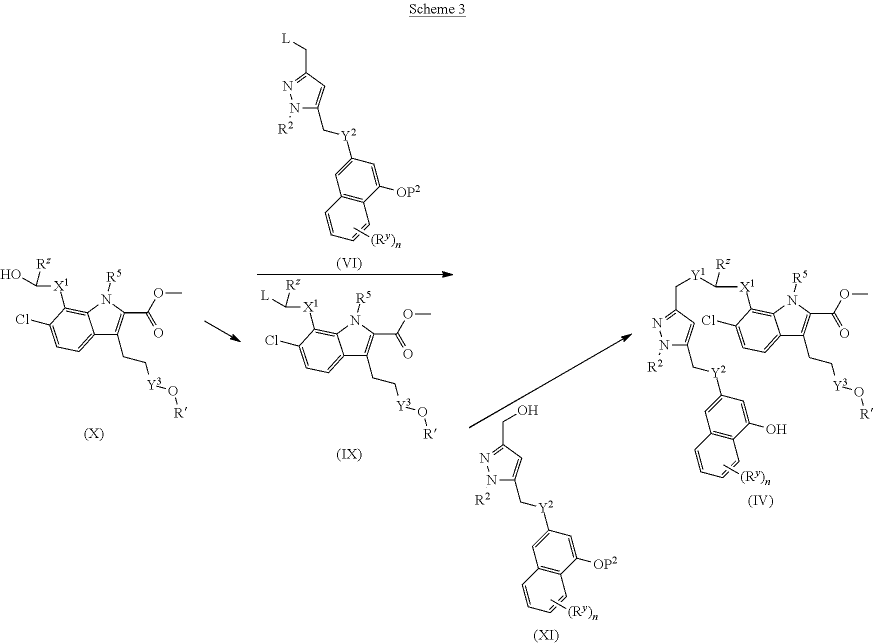

by reacting an intermediate of Formula (X) with an intermediate of Formula (VI), where L is defined as a suitable leaving group such as for example I, Br, Cl or OMs, in the presence of a suitable base such as, for example, NaH, in a suitable solvent such as, for example, THF, at a suitable temperature such as, for example, −5° C.
Alternatively, intermediates of Formula (IV) can be prepared by reacting an intermediate of Formula (IX) with an intermediate of Formula (XI), in the presence of a suitable base such as, for example, Cs₂CO₃, in a suitable solvent such as, for example, acetonitrile or methanol, or a mixture thereof, at a suitable temperature such as, for example, 50° C.
Intermediates of Formula (IX) can be prepared by reacting an intermediate of Formula (X) with a suitable activating agent such as, for example, MsCl, in presence of a suitable base such as, for example, Et₃N, in a suitable solvent such as DCM, at a suitable temperature such as, for example, room temperature.

It will be clear for somebody skilled in the art, that compounds of Formula (IV) in which R^{z} is hydrogen and Y¹ is O can also be prepared using a similar procedure starting from an intermediate of Formula (XIII).

Intermediates of Formula (X) wherein X¹, R⁵ and R^{z} are defined as in Formula (I) and Y³/R′ is CH₂/TBDMS, can be prepared according to Scheme 4,

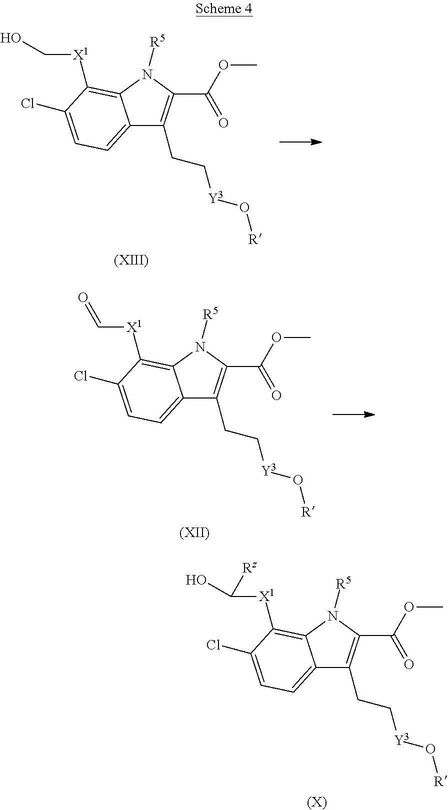

by reacting intermediates of Formula (XII) with a suitable carbon nucleophile such as, for example, an organomagnesium halide, in a suitable solvent such as, for example, THF, at a suitable temperature such as, for example, 0° C.
Intermediates of Formula (XII) can be prepared by reacting an intermediate of Formula (XIII) with a suitable oxidizing agent such as, for example, MnO₂ or Dess-Martin periodinane (3-oxo-1,3-dihydro-1 λ5,2-benziodoxole-1,1,1-triyl triacetate), in a suitable solvent such as DCM, at a suitable temperature such as, for example, room temperature.

Intermediates of Formula (XIII), wherein X¹ and R⁵ are as defined in Formula (I) and Y³/R′ is C═O/Me or Y³/R′ is CH₂/TBDMS, can be prepared according to Scheme 5,

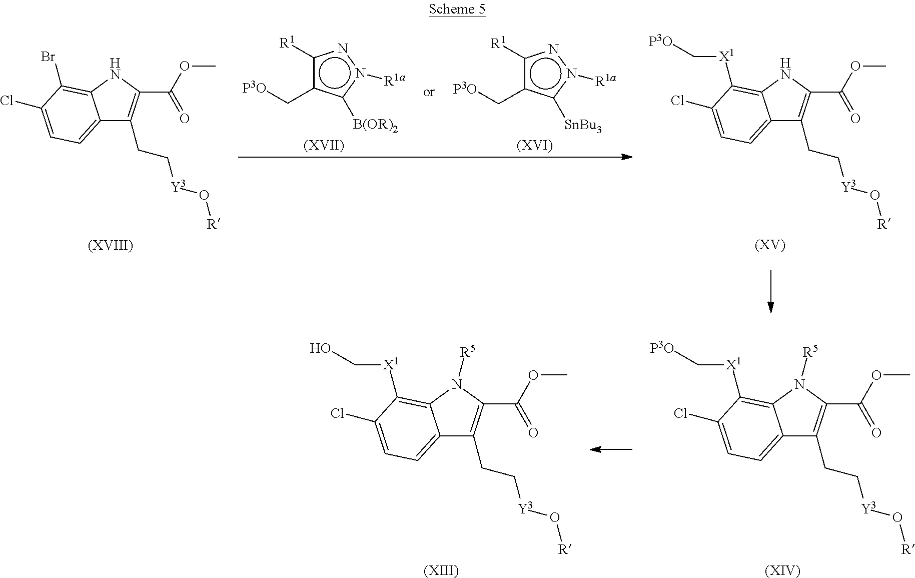

by reacting an intermediate of Formula (XIV), wherein P³ is a suitable protecting group such as, for example, PMB, with a suitable deprotecting agent such as, for example, 2,3-dichloro-5,6-dicyano-1,4-benzoquinone (DDQ), in a suitable solvent such as, for example, DCM, at a suitable temperature such as, for example, room temperature.
Intermediates of Formula (XIV) can be prepared by reacting an intermediate of Formula (XV) with a suitable alkylating reagent such as, for example, MeI (methyl iodide) or EtI (ethyl iodide), in the presence of a suitable base such as, for example, Cs₂CO₃, in a suitable solvent such as, for example, DMF, at a suitable temperature such as, for example, room temperature. It will be clear to a skilled person that when this alkylation step is not performed, R⁵ will remain a hydrogen through the remainder of the synthesis.
Intermediates of Formula (XV) wherein Y³ is C═O, and R′ is Me, can be prepared by reacting methyl 7-bromo-6-chloro-3-(3-methoxy-3-oxopropyl)-1H-indole-2-carboxylate (CAS [2143010-85-7]) with an intermediate of Formula (XVI), in the presence of a suitable catalyst such as, for example, [1,1′-Bis(di-tert-butylphosphino)ferrocene]dichloropalladium(II) (Pd(dtbpf)Cl₂), in the presence of a suitable base such as, for example, K₃PO₄, in a suitable solvent such as, for example, toluene, at a suitable temperature such as, for example, 125° C. Alternatively to an intermediate of Formula (XVI), an intermediate of Formula (XVII) can be reacted in the presence of a suitable catalyst such as, for example, [(2-Dicyclohexylphosphino-2′,6′-bis(N,N-dimethylamino)-1,1′-biphenyl)-2-(2′-amino-1,1′-biphenyl)] palladium(II) methanesulfonate (CPhos-Pd-G3) in the presence of a suitable base such as, for example, Na₂CO₃ or K₂CO₃, in a suitable solvent such as, for example, a mixture of 1,4-dioxane and water, at a suitable temperature such as, for example, 70° C.
Alternatively, this whole synthetic pathway may start from methyl 7-bromo-6-chloro-3-(3-hydroxypropyl)-1H-indole-2-carboxylate (CAS [2245716-18-9]) after its protection by a suitable protecting group reagent such as, for example, TBDMSCl (tert-butyldimethylchlorosilane), in the presence of a suitable base such as, for example, Et₃N (triethylamine) or DMAP (4-dimethylaminopyridine), or a mixture thereof, in a suitable solvent such as, for example, THF, at a suitable temperature such as, for example, room temperature, leading to intermediates (XVIII) wherein Y³ is CH₂ and R′ is a suitable protecting group such as TBDMS.

Alternatively, intermediates of Formula (XIV), wherein R⁵ is a suitable protecting group such as SEM (trimethylsilylethoxymethyl), can be prepared by reacting an intermediate of Formula (XV) with a suitable protecting group precursor such as, for example, SEMCl (2-(trimethylsilyl)ethoxymethyl chloride), in the presence of a suitable base such as, for example, NaH, in a suitable solvent such as, for example, DMF, at a suitable temperature such as, for example, 0° C. or room temperature.

Intermediates of Formula (XVI) wherein R¹ and R^{1a} are defined as in Formula (I) and P³ is a suitable protecting group such as, for example, TBDMS or PMB, can be prepared according to Scheme 6,

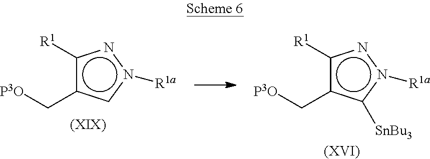

by reacting an intermediate of Formula (XIX) with a suitable trialkylstannyl halide such as, for example, tributyltin chloride (Bu₃SnCl), in the presence of a suitable base such as, for example, butyllithium (BuLi), in a suitable solvent, such as, for example, THF, at a suitable temperature such as, for example, −78° C.

Intermediates of Formula (XVII), wherein R¹ and R^{1a} are defined as in Formula (I), P³ is a suitable protecting group such as, for example, TBDMS or PMB, and B(OR)₂ represents a boronic acid or suitable boronate ester, can be prepared according to Scheme 7,

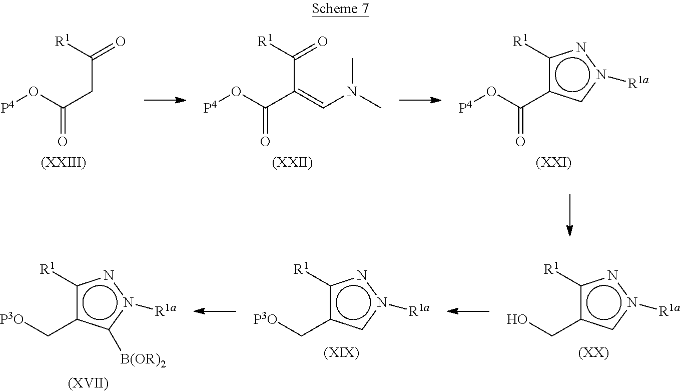

By reacting an intermediate of Formula (XIX) with a suitable boronate such as, for example, isopropoxyboronic acid pinacol ester, in the presence of a suitable base such as, for example, BuLi, in a suitable solvent, such as, for example, THF, at a suitable temperature such as, for example, −78° C.
Intermediates of Formula (XIX) can be prepared by reacting an intermediate of Formula (XX) with a suitable protecting group precursor such as, for example, TBDMSCl or PMBCl, in the presence of a suitable base such as, for example, Et₃N or DMAP, or a mixture thereof (for TBDMSCl), or NaH (for PMBCl), in a suitable solvent such as, for example, THE or DMF, at a suitable temperature such as, for example, room temperature or 0° C.
Intermediates of Formula (XX) can be prepared by reacting an intermediate of Formula (XXI) wherein P⁴ is a suitable alkyl such as, for example, methyl, with a suitable reducing agent such as, for example, LiBH₄, in a suitable solvent such as, for example, 2-methyltetrahydrofuran (2-MeTHF), at a suitable temperature such as, for example, room temperature.
Intermediates of Formula (XXI) can be prepared by reacting an intermediate of Formula (XXII) with a hydrazine R^{1a}NHNH₂ in which R^{1a} is a suitable alkyl such as, for example, methyl, in a suitable solvent such as, for example, dry diethyl ether, at a suitable temperature such as, for example, 0° C.
Intermediates of Formula (XXII) can be prepared by reacting a suitable beta-ketoester of Formula (XXIII) such as, for instance, methyl 4-(2-methoxyethoxy)-3-oxobutanone (CAS [360783-71-7]) with a suitable dialkylacetal such as, for example, 1,1-dimethoxy-N,N-dimethyl-methanamine (CAS [4637-24-5]) under suitable conditions such as, for instance, in the absence of solvent, at a suitable temperature such as, for instance, room temperature.

Intermediates of Formula (VIII) wherein (R^{y})ₙ and R² are as defined in Formula (I), and P² is a suitable protecting group such as, for example, TBDPS or PMB, can be prepared according to Scheme 8,

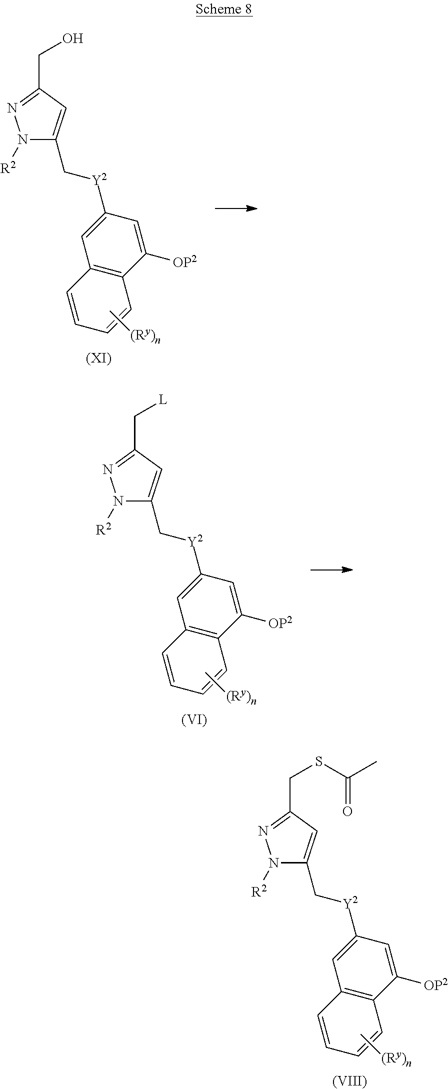

by reacting an intermediate of Formula (VI) with AcSK in a suitable solvent such as, for example, DMF, at a suitable temperature such as, for example, room temperature.
Intermediates of Formula (VI) can be prepared by reacting an intermediate of Formula (XI), in the presence of a suitable activating agent such as, for example, SOCl₂, in a suitable solvent such as, for example, DCM, at a suitable temperature such as, for example, 0° C.

Intermediates of Formula (XI), wherein (R^{y})ₙ and R² are defined as in Formula (I), P² is a suitable protecting group such as, for example, TBDPS or PMB, and Y² is CH₂, can be prepared according to Scheme 9,

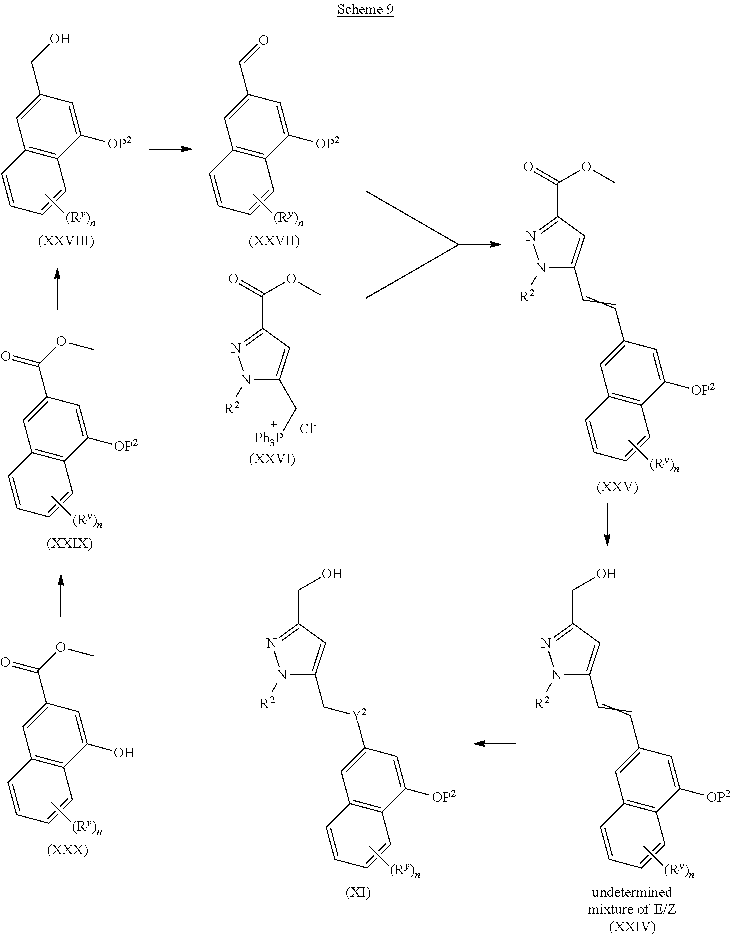

by reacting an intermediate of Formula (XXIV) with a suitable hydrogenating reagent such as, for example, hydrogen gas, in the presence of a suitable catalyst such as, for example, Pd/C, in a suitable solvent such as, for example, MeOH, at a suitable temperature such as, for example, room temperature.
Intermediates of Formula (XXIV) can be prepared by reacting an intermediate of Formula (XXV) with a suitable reducing agent such as, for example, LiAlH₄, in a suitable solvent such as, for example, THF, at a suitable temperature such as, for example, 0° C.
Intermediates of Formula (XXV) can be prepared by reacting an intermediate of Formula (XXVI) with an intermediate of Formula (XXVII), in the presence of a suitable base such as, for example, NaH, in a suitable solvent such as, for example, THF, at a suitable temperature such as, for example, −10° C.
Intermediates of Formula (XXVII) can be prepared by reacting an intermediate of Formula (XXVIII) with a suitable oxidizing agent such as, for example, MnO₂, in a suitable solvent such as, for example, acetonitrile, at a suitable temperature such as, for example, 60° C.
Intermediates of Formula (XXVIII) can be prepared by reacting an intermediate of Formula (XXIX) with a suitable reducing agent such as, for example, LiAlH₄, in a suitable solvent such as, for example, THF, at a suitable temperature such as, for example, 0° C.
Intermediates of Formula (XIX) can be prepared by reacting an intermediate of Formula (XXX) with a suitable protecting reagent such as, for example, tert-butyl(chloro)diphenylsilane (TBDPSCl) or 4-methoxybenzyl chloride (PMBCl), in the presence of a suitable base such as, for example, imidazole or NaH, in a suitable solvent such as, for example, DMF, at a suitable temperature such as, for example, room temperature.
Intermediates of Formula (XXVI) and Intermediates of Formula (XXX) are commercially available or can be prepared according to procedures described in literature.

Alternatively, an intermediate of Formula (III), wherein X¹, R², Y², R⁵, R and n are as defined in Formula (I), R^{z} is defined as H (hydrogen) and Y¹ is defined as CH₂ can be prepared according to Scheme 10,

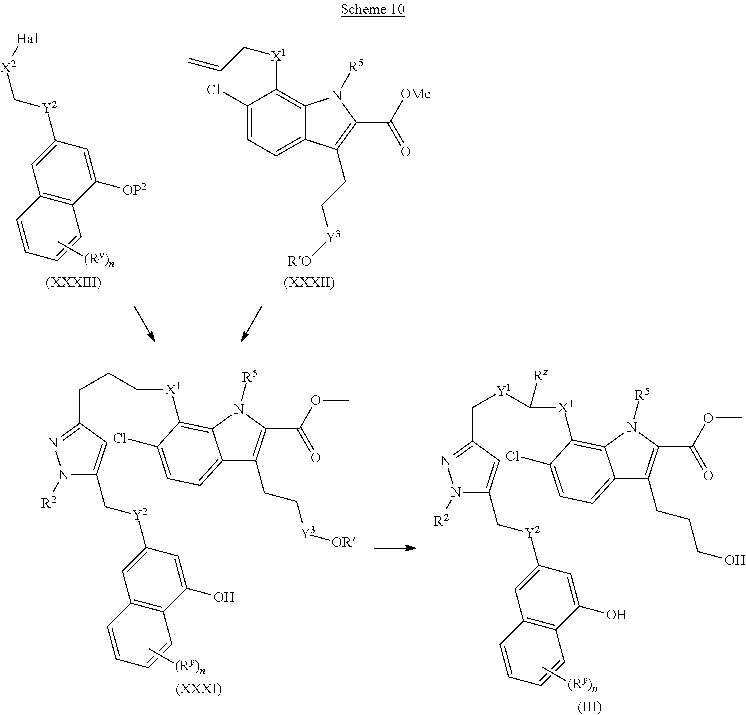

by reacting an intermediate of Formula (XXXI), wherein Y³/R′ is CH₂/TBDMS, with a suitable deprotecting agent such as, for example, pTsOH, in a suitable solvent such as, for example, MeOH, at a suitable temperature such as, for example, room temperature.
Alternatively, Intermediates of Formula (III) can be prepared by reacting an intermediate of Formula (XXXI), wherein Y³/R′ is C═O/Me, with a suitable reducing reagent such as, for example, BH₃·DMS (borane dimethylsulfide), in a suitable in a suitable solvent such as, for example, THF, at a suitable temperature such as, for example, room temperature or 50° C.
Intermediates of Formula (XXXI) can be prepared in a two-steps procedure, by first reacting an intermediate of Formula (XXXII) with a suitable borane such as, for example, 9-borabicyclo[3.3.1]nonane (9-BBN) (CAS [280-64-8]), in a suitable solvent such as, for example, THF, at a suitable temperature such as, for example, 75° C. In a second step, the resulting intermediate can be reacted with an intermediate of Formula (XXXIII), in the presence of a suitable base such as, for example, K₃PO₄, and a suitable catalyst such as, for example, dichloro[1,1′-bis(di-t-butylphosphino)ferrocene]palladium(II) Pd(dtbpf)Cl₂ (CAS [95408-45-0]), in a suitable solvent such as, for example, a mixture of THE and water, at a suitable temperature such as, for example, 80° C.

Intermediates of Formula (XXXIII), wherein X², Y², R^{y} and n are defined as in Formula (I), P² is a suitable protecting group such as, for example, TBDMS, and Hal is a suitable halide such as, for example, bromide, can be prepared according to Scheme 11,

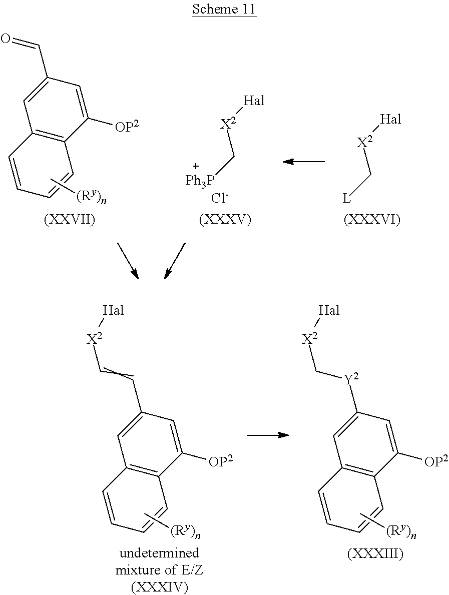

by reacting an intermediate of Formula (XXXIV) with a suitable hydrogenating agent such as, for example, hydrogen, in the presence of a suitable catalyst such as, for example, PtO₂, in a suitable solvent such as, for example, ethyl acetate (EtOAc), at a suitable temperature such as, for example, room temperature.
Intermediates of Formula (XXXIV) can be prepared by reacting an intermediate of Formula (XXXV) with an intermediate of Formula (XXVII) in the presence of a suitable base such as, for example, NaH, in a suitable solvent such as, for example, THF, at a suitable temperature such as, for example, −30° C. or 0° C.
Intermediates of Formula (XXXV) can be prepared by reacting a suitable haloheterocycle of Formula (XXXVI) such as, for example, 3-bromo-5-(chloromethyl)-1-methyl-1H-pyrazole (CAS [2109428-60-4]), with a suitable phosphine such as, for example, PPh₃, in a suitable solvent such as, for example, acetonitrile (ACN), at a suitable temperature such as, for example, 85° C.

Intermediates of Formula (XXXVI), wherein X² is defined as in Formula (I), L is a suitable leaving group such as, for example, chloride, and Hal is a suitable halide such as, for example, bromide, can be prepared according to Scheme 12,

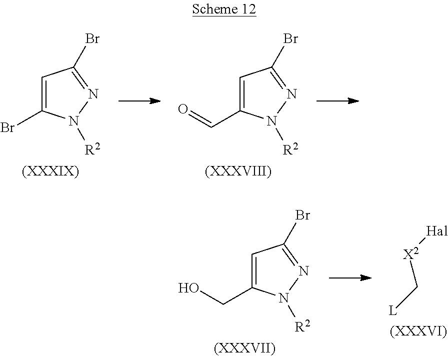

by reacting an intermediate of Formula (XXXVII) with a suitable activating agent such as, for example, thionyl chloride, in a suitable solvent such as, for example, DCM, at a suitable temperature such as, for example, room temperature.
Intermediates of Formula (XXXVII) can be prepared by reacting an intermediate of Formula (XXXVIII) with a suitable reducing agent such as, for example, NaBH₄, in a suitable solvent such as, for example, methanol (MeOH), at a suitable temperature such as, for example, 15° C.
Intermediates of Formula (XXXVIII) can be prepared by reacting an intermediate of Formula (XXXIX) with a suitable acylating agent such as, for example, DMF, in the presence of a suitable base such as, for example, BuLi, in a suitable solvent such as, for example, THF, at a suitable temperature such as, for example, −78° C.
Intermediates of Formula (XXXIX) are commercially available or can be prepared according to procedures described in literature.

Intermediates of Formula (XXXII) in which X¹, R⁵ are as in Formula (I), and Y³/R′ is CH₂/TBDMS or Y³/R′ is C═O/Me, can be prepared according to Scheme 13,

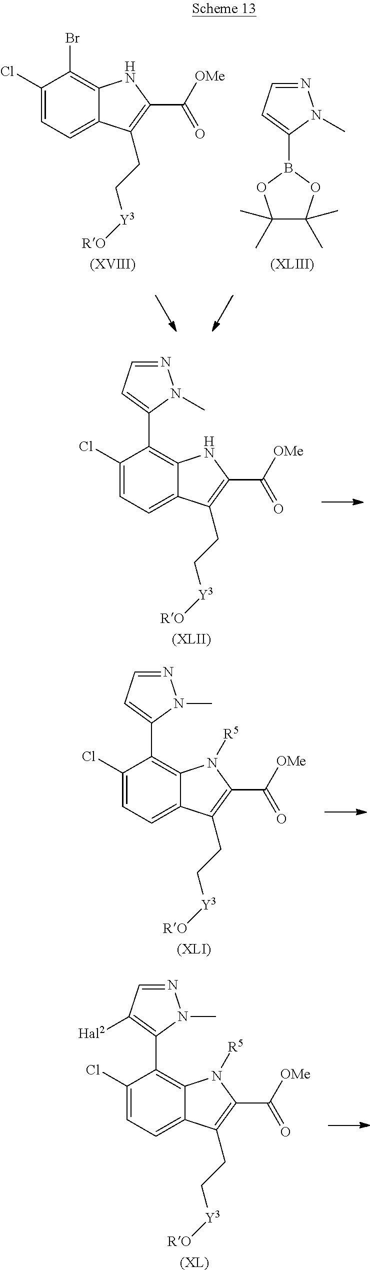

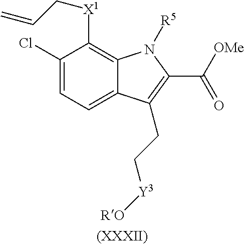

by reacting an intermediate of Formula (XL), wherein Hal² is a suitable halide such as, for example, iodide, with a suitable reagent such as, for example, allyltributyltin, in the presence of a suitable catalyst such as, for example, palladium-tetrakis(triphenylphosphine) (Pd(PPh₃)₄), in the presence of a suitable additive such as, for example, LiCl, in a suitable solvent such as, for example, DMF, at a suitable temperature such as, for example, 85° C.
Intermediates of Formula (XL) can be prepared by reacting an intermediate of Formula (XLI) with a suitable halogenating agent such as, for example, N-iodosuccinimide, in the presence of a suitable catalyst such as, for example, zinc bis(trifluoromethylsulfonyl)imide (CAS [168106-25-0]), in a suitable solvent such as, for example, DCM, at a suitable temperature such as, for example, 50° C.
Intermediates of Formula (XLI) can be prepared by reacting an intermediate of Formula (XLII) with a suitable alkylating agent such as, for example, ethyl iodide, in the presence of a suitable base such as, for example, Cs₂CO₃, in a suitable solvent such as, for example, DMF, at a suitable temperature such as, for example, 80° C.
Intermediates of Formula (XLII) can be prepared by reacting an intermediate of Formula (XVIII) with an intermediate of Formula (XLIII) (CAS [847818-74-0]), in the presence of a suitable base such as, for example, K₂CO₃, in the presence of a suitable catalyst such as, for example, bis(di-tert-butyl(4-dimethylaminophenyl)phosphine)dichloropalladium (II), in a suitable solvent such as, for example, a mixture of 1,4-dioxane and water, at a suitable temperature such as, for example, 90° C.

Alternatively, intermediates of Formula (XI), wherein (R^{y})ₙ and R² are defined as in Formula (I), Y² is CH₂CH₂, and P² is a suitable protecting group such as, for example, TBDPS or PMB, can be prepared according to Scheme 14,

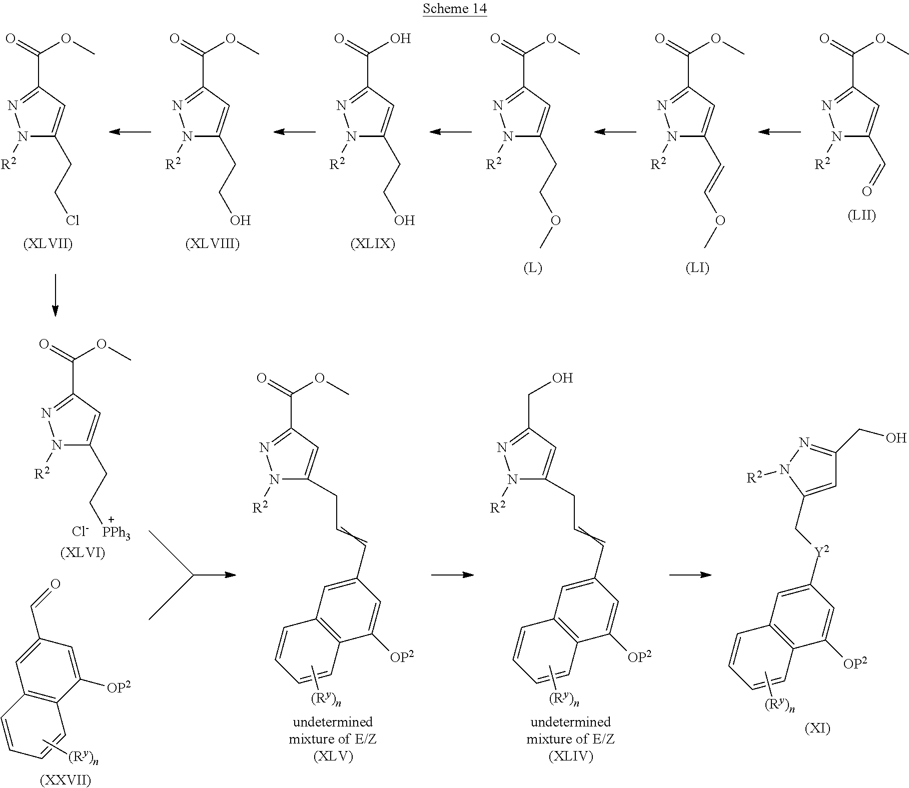

by reacting an intermediate of Formula (XLIV) with a suitable hydrogenating reagent such as, for example, hydrogen gas, in the presence of a suitable catalyst such as, for example, Pd/C, in a suitable solvent such as, for example, MeOH, at a suitable temperature such as, for example, room temperature.
Intermediates of Formula (XLIV) can be prepared by reacting an intermediate of Formula (XLV) with a suitable reducing agent such as, for example, LiAlH₄, in a suitable solvent such as, for example, THF, at a suitable temperature such as, for example, 0° C.
Intermediates of Formula (XLV) can be prepared by reacting an intermediate of Formula (XLVI) with an intermediate of Formula (XXVII), in the presence of a suitable base such as, for example, NaH, in a suitable solvent such as, for example, THF, at a suitable temperature such as, for example, −10° C.
Intermediates of Formula (XLVI) can be prepared by reacting an intermediate of Formula (XLVII) with a suitable phosphine such as, for example, PPh₃, in a suitable solvent such as, for example, DCM, at a suitable temperature such as, for example, room temperature.
Intermediates of Formula (XLVII) can be prepared by reacting an intermediate of Formula (XLVIII) with a suitable activating agent such as, for example SOCl₂, in a suitable solvent such as, for example, DCM, at a suitable temperature such as, for example, room temperature.
Intermediates of Formula (XLVIII) can be prepared by reacting an intermediate of Formula (XLIX) with a suitable alcohol such as, for example, MeOH, in the presence of a suitable acid, such as, for example, sulfuric acid, at a suitable temperature such as, for example, room temperature.
Intermediates of Formula (XLIX) can be prepared by reacting an intermediate of Formula (L) with a suitable demethylating agent such as, for example, boron tribromide, in a suitable solvent such as, for example, DCM, at a suitable temperature such as, for example, −78° C. or 0° C.
Intermediates of Formula (L) can be prepared by reacting an intermediate of Formula (LI) with a suitable reducing agent such as, for example, hydrogen gas, in the presence of a suitable catalyst such as, for example, Pd/C, in a suitable solvent such as, for example, MeOH, at a suitable temperature such as, for example, room temperature.
Intermediates of Formula (LI) can be prepared by reacting an intermediate of Formula (LII) with a suitable vinylating reagent such as, for example, (methoxymethyl)triphenylphosphonium chloride (CAS [4009-98-7]), in the presence of a suitable base such as, for example, potassium tert-butoxide (tBuOK), in a suitable solvent such as, for example, THF, at a suitable temperature such as, for example, 0° C. or room temperature.
Intermediates of Formula (LII) can be prepared according to procedures described in literature.

It will be appreciated that where appropriate functional groups exist, compounds of various formulae or any intermediates used in their preparation may be further derivatized by one or more standard synthetic methods employing condensation, substitution, oxidation, reduction, or cleavage reactions. Particular substitution approaches include conventional alkylation, arylation, heteroarylation, acylation, sulfonylation, halogenation, nitration, formylation and coupling procedures.

The compounds of Formula (I) may be synthesized in the form of racemic mixtures of enantiomers which can be separated from one another following art-known resolution procedures. The racemic compounds of Formula (I) containing a basic nitrogen atom may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (Boc), benzyloxycarbonyl (CBz) and 9-fluorenylmethyleneoxycarbonyl (Fmoc). The need for such protection is readily determined by one skilled in the art. For a general description of protecting groups and their use, see T. W. Greene and P. G. M. Wuts, Protective Groups in Organic Synthesis, 4th ed., Wiley, Hoboken, N.J., 2007.

### Pharmacology of Compounds

It has been found that the compounds of the present invention inhibit one of more MCL-1 activities, such as MCL-1 antiapoptotic activity.

An MCL-1 inhibitor is a compound that blocks one or more MCL-1 functions, such as the ability to bind and repress proapoptotic effectors Bak and Bax or BH3 only sensitizers such as Bim, Noxa or Puma.

The compounds of the present invention can inhibit the MCL-1 pro-survival functions. Therefore, the compounds of the present invention may be useful in treating and/or preventing, in particular treating, diseases that are susceptible to the effects of the immune system such as cancer.

In another embodiment of the present invention, the compounds of the present invention exhibit anti-tumoral properties, for example, through immune modulation.

In an embodiment, the present invention is directed to methods for treating and/or preventing a cancer, wherein the cancer is selected from those described herein, comprising administering to a subject in need thereof (preferably a human), a therapeutically effective amount of a compound of Formula (I), or pharmaceutically acceptable salt, or a solvate thereof.

In an embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of acute lymphoblastic leukemia (ALL), acute myeloid leukemia (AML), B cells acute lymphoblastic leukemia, B-cell chronic lymphocytic leukemia (CLL), bladder cancer, breast cancer, chronic lymphocytic leukemia, chronic myeloid leukemia, colon adenocarcinoma, diffuse large B cell lymphoma, esophageal cancer, follicular lymphoma, gastric cancer, head and neck cancer (including, but not limited to head and neck squamous cell carcinoma), hematopoietic cancer, hepatocellular carcinoma, Hodgkin lymphoma, liver cancer, lung cancer (including but not limited to lung adenocarcinoma), lymphoma, medulloblastoma, melanoma, monoclonal gammopathy of undetermined significance, multiple myeloma, myelodysplastic syndromes, myelofibrosis, myeloproliferative neoplasms, ovarian cancer, ovarian clear cell carcinoma, ovarian serous cystadenoma, pancreatic cancer, polycythemia vera, prostate cancer, rectum adenocarcinoma, renal cell carcinoma, smoldering multiple myeloma, T cell acute lymphoblastic leukemia, T cell lymphoma, and Waldenstroms macroglobulinemia.

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is preferably selected from the group consisting of acute lymphoblastic leukemia (ALL), acute myeloid leukemia (AML), B cells acute lymphoblastic leukemia, B-cell chronic lymphocytic leukemia (CLL), breast cancer, chronic lymphocytic leukemia, chronic myeloid leukemia, diffuse large B cell lymphoma, follicular lymphoma, hematopoietic cancer, Hodgkin lymphoma, lung cancer (including, but not limited to lung adenocarcinoma) lymphoma, monoclonal gammopathy of undetermined significance, multiple myeloma, myelodysplastic syndromes, myelofibrosis, myeloproliferative neoplasms, smoldering multiple myeloma, T cell acute lymphoblastic leukemia, T cell lymphoma and Waldenstroms macroglobulinemia.

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of adenocarcinoma, benign monoclonal gammopathy, biliary cancer (including, but not limited to, cholangiocarcinoma), bladder cancer, breast cancer (including, but not limited to, adenocarcinoma of the breast, papillary carcinoma of the breast, mammary cancer, medullary carcinoma of the breast), brain cancer (including, but not limited to, meningioma), glioma (including, but not limited to, astrocytoma, oligodendroglioma; medulloblastoma), bronchus cancer, cervical cancer (including, but not limited to, cervical adenocarcinoma), chordoma, choriocarcinoma, colorectal cancer (including, but not limited to, colon cancer, rectal cancer, colorectal adenocarcinoma), epithelial carcinoma, endothelial sarcoma (including, but not limited to, Kaposi's sarcoma, multiple idiopathic hemorrhagic sarcoma), endometrial cancer (including, but not limited to, uterine cancer, uterine sarcoma), esophageal cancer (including, but not limited to, adenocarcinoma of the esophagus, Barrett's adenocarinoma), Ewing sarcoma, gastric cancer (including, but not limited to, stomach adenocarcinoma), gastrointestinal stromal tumor (GIST), head and neck cancer (including, but not limited to, head and neck squamous cell carcinoma), hematopoietic cancers (including, but not limited to, leukemia such as acute lymphocytic leukemia (ALL) (including, but not limited to, B-cell ALL, T-cell ALL), acute myelocytic leukemia (AML) (e.g. B-cell AML, T-cell AML), chronic myelocytic leukemia (CML) (e.g. B-cell CML, T-cell CML), and chronic lymphocytic leukemia (CLL) (e.g. B-cell CLL, T-cell CLL), lymphoma such as Hodgkin lymphoma (HL) (including, but not limited to, B-cell HL, T-cell HL) and non-Hodgkin lymphoma (NHL) (e.g. B-cell NHL such as diffuse large cell lymphoma (DLCL) (e.g. diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma. splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g. cutaneous T-cell lymphoma (CTCL) (including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, multiple myeloma (MM), heavy chain disease (including, but not limited to, alpha chain disease, gamma chain disease, mu chain disease), immunocytic amyloidosis, kidney cancer (including, but not limited to, nephroblastoma a.k.a. Wilms' tumor, renal cell carcinoma), liver cancer (including, but not limited to, hepatocellular cancer (HCC), malignant hepatoma), lung cancer (including, but not limited to, bronchogenic carcinoma, non-small cell lung cancer (NSCLC), squamous lung cancer (SLC), adenocarcinoma of the lung, Lewis lung carcinoma, lung neuroendocrine tumors, typical carcinoid, atypical carcinoid, small cell lung cancer (SCLC), and large cell neuroendocrine carcinoma), myelodysplastic syndromes (MDS), myeloproliferative disorder (MPD), polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES), ovarian cancer (including, but not limited to, cystadenocarcinoma, ovarian embryonal carcinoma, ovarian adenocarcinoma), pancreatic cancer (including, but not limited to, pancreatic andenocarcinoma, intraductal papillary mucinous neoplasm (IPMN), Islet cell tumors), prostate cancer (including, but not limited to, prostate adenocarcinoma), skin cancer (including, but not limited to, squamous cell carcinoma (SCC), keratoacanthoma (KA), melanoma, basal cell carcinoma (BCC)) and soft tissue sarcoma (e.g. malignant fibrous histiocytoma (MFH), liposarcoma, malignant peripheral nerve sheath tumor (MPNST), chondrosarcoma, fibrosarcoma, myxosarcoma).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of benign monoclonal gammopathy, breast cancer (including, but not limited to, adenocarcinoma of the breast, papillary carcinoma of the breast, mammary cancer, medullary carcinoma of the breast), hematopoietic cancers (including, but not limited to, leukemia such as acute lymphocytic leukemia (ALL) (including, but not limited to, B-cell ALL, T-cell ALL), acute myelocytic leukemia (AML) (e.g. B-cell AMIL, T-cell AML), chronic myelocytic leukemia (CML) (e.g. B-cell CML, T-cell CML), and chronic lymphocytic leukemia (CLL) (e.g. B-cell CLL, T-cell CLL), lymphoma such as Hodgkin lymphoma (HL) (including, but not limited to, B-cell HL, T-cell HL) and non-Hodgkin lymphoma (NHL) (e.g. B-cell NHL such as diffuse large cell lymphoma (DLCL) (e.g. diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma. splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g. cutaneous T-cell lymphoma (CTCL) (including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, multiple myeloma (MM), heavy chain disease (including, but not limited to, alpha chain disease, gamma chain disease, mu chain disease), immunocytic amyloidosis, liver cancer (including, but not limited to, hepatocellular cancer (HCC), malignant hepatoma), lung cancer (including, but not limited to, bronchogenic carcinoma, non-small cell lung cancer (NSCLC), squamous lung cancer (SLC), adenocarcinoma of the lung, Lewis lung carcinoma, lung neuroendocrine tumors, typical carcinoid, atypical carcinoid, small cell lung cancer (SCLC), and large cell neuroendocrine carcinoma), myelodysplastic syndromes (MDS), myeloproliferative disorder (MPD), and prostate cancer (including, but not limited to, prostate adenocarcinoma).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of prostate, lung, pancreatic, breast, ovarian, cervical, melanoma, B-cell chronic lymphocytic leukemia (CLL), acute myeloid leukemia (AML), and acute lymphoblastic leukemia (ALL).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is multiple myeloma.

The compounds according to the present invention or pharmaceutical compositions comprising said compounds, may also have therapeutic applications in combination with immune modulatory agents, such as inhibitors of the PD1/PDL1 immune checkpoint axis, for example antibodies (or peptides) that bind to and/or inhibit the activity of PD-1 or the activity of PD-L1 and or CTLA-4 or engineered chimeric antigen receptor T cells (CART) targeting tumor associated antigens.

The compounds according to the present invention or pharmaceutical compositions comprising said compounds, may also be combined with radiotherapy or chemotherapeutic agents (including, but not limited to, anti-cancer agents) or any other pharmaceutical agent which is administered to a subject having cancer for the treatment of said subject's cancer or for the treatment or prevention of side effects associated with the treatment of said subject's cancer.

The compounds according to the present invention or pharmaceutical compositions comprising said compounds, may also be combined with other agents that stimulate or enhance the immune response, such as vaccines.

In an embodiment, the present invention is directed to methods for treating and/or preventing a cancer (wherein the cancer is selected from those described herein) comprising administering to a subject in need thereof (preferably a human), a therapeutically effective amount of co-therapy or combination therapy; wherein the co-therapy or combination therapy comprises a compound of Formula (I) of the present invention and one or more anti-cancer agent(s) selected from the group consisting of (a) immune modulatory agent (such as inhibitors of the PD1/PDL1 immune checkpoint axis, for example antibodies (or peptides) that bind to and/or inhibit the activity of PD-1 or the activity of PD-L1 and or CTLA-4); (b) engineered chimeric antigen receptor T cells (CART) targeting tumor associated antigens; (c) radiotherapy; (d) chemotherapy; and (e) agents that stimulate or enhance the immune response, such as vaccines.

The present invention is directed to compounds of Formula (I) and pharmaceutically acceptable salts, and solvates thereof, for use as a medicament.

The present invention is directed to compounds of Formula (I) and pharmaceutically acceptable salts, and solvates thereof, for use in the inhibition of MCL-1 activity.

As used herein, unless otherwise noted, the term “anti-cancer agents” shall encompass “anti-tumor cell growth agents” and “anti-neoplastic agents”.

The present invention is directed to compounds of Formula (I) and pharmaceutically acceptable salts, and solvates thereof, for use in treating and/or preventing diseases (preferably cancers) mentioned above.

The present invention is directed to compounds of Formula (I) and pharmaceutically acceptable salts, and solvates thereof, for treating and/or preventing diseases (preferably cancers) mentioned above.

The present invention is directed to compounds of Formula (I) and pharmaceutically acceptable salts, and solvates thereof, for treating and/or preventing, in particular for treating, a disease, preferably a cancer, as described herein (for example, multiple myeloma).

The present invention is directed to compounds of Formula (I) and pharmaceutically acceptable salts, and solvates thereof, for use in treating and/or preventing, in particular for treating, a disease, preferably a cancer, as described herein (for example, multiple myeloma).

The present invention is directed to compounds of Formula (I) and pharmaceutically acceptable salts, and solvates thereof, for treating and/or preventing, in particular for treating, MCL-1 mediated diseases or conditions, preferably cancer, more preferably a cancer as herein described (for example, multiple myeloma).

The present invention is directed to compounds of Formula (I) and pharmaceutically acceptable salts, and solvates thereof, for use in treating and/or preventing, in particular for use in treating, MCL-1 mediated diseases or conditions, preferably cancer, more preferably a cancer as herein described (for example, multiple myeloma).

The present invention relates to compounds of Formula (I) and pharmaceutically acceptable salts, and solvates thereof, for the manufacture of a medicament.

The present invention relates to compounds of Formula (I) and pharmaceutically acceptable salts, and solvates thereof, for the manufacture of a medicament for the inhibition of MCL-1.

The present invention relates to compounds of Formula (I) and pharmaceutically acceptable salts, and solvates thereof, for the manufacture of a medicament for treating and/or preventing, in particular for treating, a cancer, preferably a cancer as herein described. More particularly, the cancer is a cancer which responds to inhibition of MCL-1 (for example, multiple myeloma).

The present invention is directed to compounds of Formula (I) and pharmaceutically acceptable salts, and solvates thereof, for the manufacture of a medicament for treating and/or preventing, in particular for treating, any one of the disease conditions mentioned hereinbefore.

The present invention is directed to compounds of Formula (I) and pharmaceutically acceptable salts, and solvates thereof, for the manufacture of a medicament for treating and/or preventing any one of the disease conditions mentioned hereinbefore.

The compounds of Formula (I) and pharmaceutically acceptable salts, and solvates thereof, can be administered to subjects, preferably humans, for treating and/or preventing of any one of the diseases mentioned hereinbefore.

In view of the utility of the compounds of Formula (I) and pharmaceutically acceptable salts, and solvates thereof, there is provided a method of treating subjects, preferably mammals such as humans, suffering from any of the diseases mentioned hereinbefore; or a method of slowing the progression of any of the diseases mentioned hereinbefore in subject, humans; or a method of preventing subjects, preferably mammals such as humans, from suffering from any one of the diseases mentioned hereinbefore.

Said methods comprise the administration, i.e. the systemic or topical administration, preferably oral or intravenous administration, more preferably oral administration, of an effective amount of a compound of Formula (I) or a pharmaceutically acceptable salt, or a solvate thereof, to subjects such as humans.

One skilled in the art will recognize that a therapeutically effective amount of the compounds of the present invention is the amount sufficient to have therapeutic activity and that this amount varies inter alias, depending on the type of disease, the concentration of the compound in the therapeutic formulation, and the condition of the patient. In an embodiment, a therapeutically effective daily amount may be from about 0.005 mg/kg to 100 mg/kg.

The amount of a compound according to the present invention, also referred to herein as the active ingredient, which is required to achieve a therapeutic effect may vary on case-by-case basis, for example with the specific compound, the route of administration, the age and condition of the recipient, and the particular disorder or disease being treated. The methods of the present invention may also include administering the active ingredient on a regimen of between one and four intakes per day. In these methods of the present invention, the compounds according to the invention are preferably formulated prior to administration.

The present invention also provides compositions for treating and/or preventing the disorders (preferably a cancer as described herein) referred to herein. Said compositions comprise a therapeutically effective amount of a compound of Formula (I), or a pharmaceutically acceptable salt, or a solvate thereof, and a pharmaceutically acceptable carrier or diluent.

While it is possible for the active ingredient (e.g. a compound of the present invention) to be administered alone, it is preferable to administer it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of the present invention may be prepared by any methods well known in the art of pharmacy, for example, using methods such as those described in, for example, Gennaro et al. Remington's Pharmaceutical Sciences (18^{th} ed., Mack Publishing Company, 1990, see especially Part 8: Pharmaceutical preparations and their Manufacture).

The compounds of the present invention may be administered alone or in combination with one or more additional therapeutic agents. Combination therapy includes administration of a single pharmaceutical dosage formulation which contains a compound according to the present invention and one or more additional therapeutic agents, as well as administration of the compound according to the present invention and each additional therapeutic agent in its own separate pharmaceutical dosage formulation.

Therefore, in an embodiment, the present invention is directed to a product comprising, as a first active ingredient a compound according to the invention and as further, as an additional active ingredient one or more anti-cancer agent(s), as a combined preparation for simultaneous, separate or sequential use in the treatment of patients suffering from cancer.

The one or more other anti-cancer agents and the compound according to the present invention may be administered simultaneously (e.g. in separate or unitary compositions) or sequentially, in either order. In an embodiment, the two or more compounds are administered within a period and/or in an amount and/or a manner that is sufficient to ensure that an advantageous or synergistic effect is achieved. It will be appreciated that the preferred method and order of administration and the respective dosage amounts and regimes for each component of the combination will depend on the particular other anti-cancer agent and the compound of the present invention being administered, their route of administration, the particular condition, in particular tumor, being treated and the particular host being treated.

The following examples further illustrate the present invention.

### EXAMPLES

Several methods for preparing the Compounds of this invention are illustrated in the following examples. Unless otherwise noted, all starting materials were obtained from commercial suppliers and used without further purification, or alternatively can be synthesized by a skilled person by using well-known methods.

| Abbreviation | Meaning |
|---|---|
| 9-BBN | 9-borabicyclo[3.3.1]nonane |
| ADD or ADDP | 1,1′-(azodicarbonyl)dipiperidine |
| ACN | Acetonitrile |
| Celite ® | diatomaceous earth |
| Co | Compound |
| Co. No. | Compound Number |
| CPhos-Pd-G3 | [(2-dicyclohexylphosphino-2′,6′-bis(N,N- |
| | dimethylamino) -1,1′-biphenyl)-2-(2′-amino- |
| | 1,1′-biphenyl)] palladium(II) methanesulfonate |
| DCM | dichloromethane |
| DDQ | 2,3-Dichloro-5,6-dicyano-1,4-benzoquinone |
| Dicalite ® | diatomaceous earth |
| DIPE | di-isopropylether |
| DIPEA | N,N-diisopropylethylamine |
| DMAP | 4-(dimethylamino)pyridine |
| DMF | N,N-dimethylformamide |
| DMF-DMA | N,N-dimethylformamide dimethyl acetal |
| DTBAD | di-tert-butyl Azodicarboxylate |
| eq. | equivalent(s) |
| Et₂O | diethyl ether |
| EtOAc | ethyl acetate |
| EtOH | ethanol |
| g | gram(s) |
| h | hour(s) |
| HPLC | high performance liquid chromatography |
| iPrNH₂ | isopropylamine |
| iPrOH | 2-propanol |
| M | molar |
| Me | methyl |
| mg | milligram(s) |
| MeI | methyl iodide |
| mL | milliliter(s) |
| mmol | millimole(s) |
| MeOH | methanol |
| min | minute(s) |
| MP | melting point |
| MsCl | methanesulfonyl chloride |
| Ms₂O | methanesulfonic acid anhydride |
| N | normal |
| BuLi | butyllithium |
| Palladacycle | methanesulfonato(2-dicyclohexylphosphino- |
| Gen. 3 | 2′,6′-bis(dimethylamino)-1,1′-biphenyl)(2′- |
| | amino-1,1′-biphenyl-2-yl)palladium(II) |
| | CAS [1447963-73-6] |
| Pd(PPh₃)₄ | palladium-tetrakis(triphenylphosphine) |
| PBu₃ or n-Bu₃P | tributylphosphine |
| Pd/C | palladium on carbon |
| Pd(dtbpf)Cl₂ or | 1,1′-Bis (di-t-butylphosphino)ferrocene |
| PdCl₂(dtbpf) | palladium dichloride |
| PPh₃ | triphenylphosphine |
| pTsOH | p-toluenesulfonic acid |
| PTSA•H₂O | p-toluenesulfonic acid monohydrate |
| rac | racemic |
| Rochelle salt | potassium sodium tartrate tetrahydrate |
| RP | reversed phase |
| SFC | supercritical fluid chromatography |
| TBAF | tetrabutylammonium fluoride |
| TBDMSCl | tert-butyl(chloro)dimethylsilane |
| TBDPSCl | tert-butyl(chloro)diphenylsilane |
| TFA | trifluoroacetic acid |
| THF | tetrahydrofuran |

As understood by a person skilled in the art, Compounds synthesized using the protocols as indicated may contain residual solvent or minor impurities.

A skilled person will realize that, even where not mentioned explicitly in the experimental protocols below, typically after a column chromatography purification, the desired fractions were collected and the solvent was evaporated.

In case no stereochemistry is indicated, this means it is a mixture of stereoisomers, unless otherwise is indicated or is clear from the context.

### Preparation of Intermediates

For intermediates that were used in a next reaction step as a crude or as a partially purified intermediate, in some cases no mol amounts are mentioned for such intermediate in the next reaction step or alternatively estimated mol amounts or theoretical mol amounts for such intermediate in the next reaction step are indicated in the reaction protocols described below.

### Intermediate 1

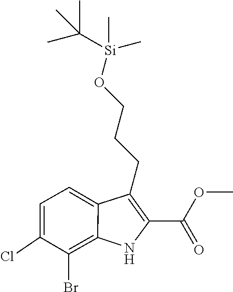 [SMILES: COC(=O)c1[nH]c2c(Br)c(Cl)ccc2c1CCCO[Si](C)(C)C(C)(C)C]

Tert-butyldimethylsilyl chloride (2.06 g, 1.4 eq.) was added portionwise to a mixture of methyl 7-bromo-6-chloro-3-(3-hydroxypropyl)-1H-indole-2-carboxylate [2245716-18-9] (3.5 g, 9.78 mmol) and imidazole (1 g, 1.5 eq.) in DCM (80 mL) at 0° C. DMAP (59 mg, 0.05 eq.) was then added and the reaction mixture was stirred at room temperature for 1 h. The reaction mixture was diluted with DCM and washed with water. The organic layer was separated, dried on MgSO₄, filtered, and evaporated to give Intermediate 1 (4.46 g, 87% yield), used without further purification.

### Intermediate 2

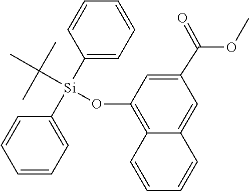 [SMILES: COC(=O)c1cc(O[Si](c2ccccc2)(c2ccccc2)C(C)(C)C)c2ccccc2c1]

TBDPSCl (6.412 mL, 1.25 eq.) was added dropwise to a solution of methyl 4-hydroxy-2-naphthoate (CAS [34205-71-5], 4 g, 19.78 mmol) and imidazole (2.35 g, 1.75 eq.) in DMF (70 mL), cooled to 0° C. Once the addition was complete, the reaction was stirred at room temperature for 14 h. The reaction mixture was diluted with EtOAc (40 mL) and washed subsequently with water, dilute aqueous HCl (0.1 M), saturated aqueous NaHCO₃ and brine (each 30 mL). The organic layer was dried over MgSO₄, filtered, and concentrated. The residue was purified by column chromatography on silica gel (heptane:EtOAc—1:0 to 9:1) to afford Intermediate 2 (8.81 g, yield: 91%) as a yellow oil.

### Intermediate 3

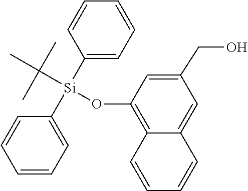 [SMILES: CC(C)(C)[Si](Oc1cc(CO)cc2ccccc12)(c1ccccc1)c1ccccc1]

LiAlH₄ (2 M solution in THF, 9.44 mL, 1.05 eq.) was slowly added to a solution of Intermediate 2 (8.8 g, 17.97 mmol) in THF (70 mL) cooled to 0° C. Once the addition was complete the reaction mixture was stirred at 0° C. for 30 min. The reaction was quenched by slow addition of EtOAc (20 mL) followed by a saturated solution of Rochelle salt. The heterogeneous mixture was stirred at room temperature for 2 h. The aqueous layer was extracted with EtOAc (2×65 mL), and the combined organic extracts were washed with brine (20 mL), dried over MgSO₄, filtered, and concentrated. The residue was purified by flash column chromatography on silica gel (heptane:EtOAc—1:0 to 3:1) to give Intermediate 3 (5.81 g, yield: 74%) as a white solid.

### Intermediate 4

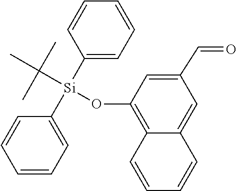 [SMILES: CC(C)(C)[Si](Oc1cc(C=O)cc2ccccc12)(c1ccccc1)c1ccccc1]

MnO₂ (5.81 g, 5 eq.) was added to a solution of Intermediate 3 (5.81 g, 13.38 mmol) in ACN (60 mL) at room temperature. The resulting solution was stirred at 60° C. for 2 h. The reaction mixture was filtered over a pad of Dicalite and concentrated to give Intermediate 4 (5.47 g, yield: 94%) as a white solid, used without further purification.

### Intermediate 5

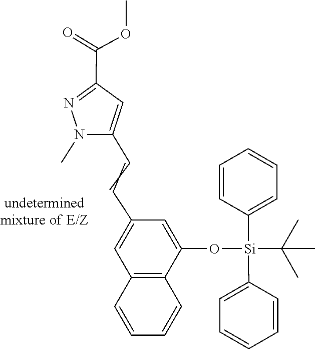 [SMILES: COC(=O)c1cc(C=Cc2cc(O[Si](c3ccccc3)(c3ccccc3)C(C)(C)C)c3ccccc3c2)n(C)n1]

NaH (653 mg, 1.1 eq.) was added to a suspension of Intermediate 73 (8.094 g, 1.1 eq.) in TIE (90 mL) at 0° C. The resulting solution (solution A) was stirred at 0° C. for 45 min before it was cooled to −25° C. A solution of Intermediate 4 (6.7 g, 15.5 mmol) in THF (16 mL) was added slowly to solution A while maintaining the temperature between −20° C. and −30° C. Once the addition was complete, the reaction was stirred at −10° C. for 1 h. The reaction was quenched by slow addition of saturated aqueous NH₄Cl (10 mL) at −10° C. and was diluted with EtOAc (100 mL). The layers were separated and the aqueous layer was extracted with EtOAc (2×100 mL). The combined organic layer was dried over MgSO₄, filtered, and concentrated. The residue was purified by column chromatography on silica gel (heptane:EtOAc—1:0 to 7:3) to afford Intermediate 5 (6.75 g, yield: 75%) as a white foam.

### Intermediate 6

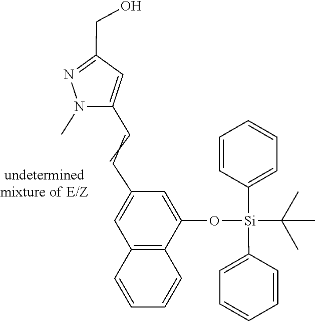 [SMILES: Cn1nc(CO)cc1C=Cc1cc(O[Si](c2ccccc2)(c2ccccc2)C(C)(C)C)c2ccccc2c1]

LiAlH₄ (2 M solution in THF, 6.1 mL, 1.05 eq.) was slowly added to a solution of Intermediate 5 (6.7 g, 11.64 mmol) in THF (45 mL) cooled to 0° C. Once the addition was complete, the reaction mixture was stirred at 0° C. for 30 min. The reaction was quenched by slow addition of EtOAc (20 mL) followed by a saturated solution of Rochelle salt. The heterogeneous mixture was stirred at room temperature for 2 h. The aqueous layer was extracted with EtOAc (2×65 mL), and the combined organic extracts were washed with brine (20 mL), dried over MgSO₄, filtered, and concentrated to afford Intermediate 6 (6.01 g, yield: 94%) as a white foam, used without further purification.

### Intermediate 7

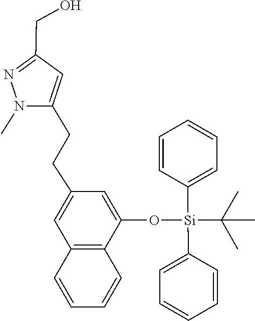 [SMILES: Cn1nc(CO)cc1CCc1cc(O[Si](c2ccccc2)(c2ccccc2)C(C)(C)C)c2ccccc2c1]

Intermediate 6 (5.95 g, 10.89 mmol) was dissolved in MeOH (280 mL). Pd/C (10%, 1159 mg, 0.1 eq.) was added under nitrogen atmosphere. The reaction mixture was then flushed with hydrogen gas and vacuum (3 times), then hydrogen (atmospheric pressure, 244 mL, 1 eq.) was taken up while stirring at room temperature. The reaction mixture was filtered over a pad of Dicalite and concentrated to give Intermediate 7 (5.9 g, yield: 98%) as a glassy yellow solid, used without further purification.

### Intermediate 8

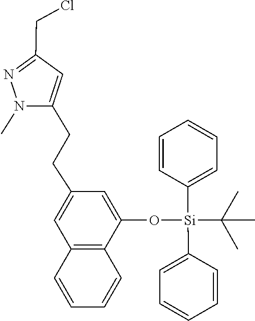 [SMILES: Cn1nc(CCl)cc1CCc1cc(O[Si](c2ccccc2)(c2ccccc2)C(C)(C)C)c2ccccc2c1]

Thionyl chloride (459 μL, 1.15 eq.) was added to a solution of Intermediate 7 (3 g, 5.47 mmol) in DCM (23 mL) cooled to 0° C. Once the addition was complete, the reaction was allowed to warm to room temperature and was stirred for 1 h. The reaction was diluted with DCM (35 mL), washed with saturated aqueous NaHCO₃ (2×50 mL) and brine (50 mL). The organic layer was dried over MgSO₄, filtered, and concentrated. The residue was purified by flash column chromatography on silica gel (heptane/EtOAc—1:0 to 8:2) to give Intermediate 8 (2.65 g, yield: 85%) as a colorless oil that crystallized on standing to a white amorphous solid.

### Intermediate 9

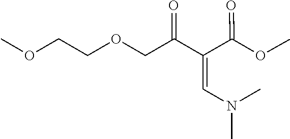

Methyl 4-(2-Methoxyethoxy)-3-oxobutanone (CAS [360783-71-7]) (6.29 g, 33.07 mmol) was mixed with 1,1-dimethoxy-N,N-dimethyl-methanamine (CAS [4637-24-5]) (4.92 g, 1.25 eq.) and stirred at room temperature for 2 h to afford Intermediate 9 (10 g, assumed quantitative), used without further purification.

### Intermediate 10

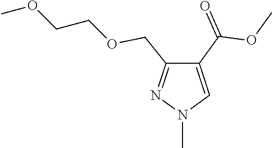

A solution of methylhydrazine (2.14 mL, 1 eq.) in dry Et₂O (120 mL) was added dropwise to a stirred solution of Intermediate 9 (10 g, 40.70 mmol) in dry Et₂O (20 mL) at 0° C. The reaction mixture was stirred at 0° C. for 15 min and then allowed to warm up to room temperature and stirred overnight. The reaction mixture was concentrated under reduced pressure and the residue was purified by flash column chromatography on silica gel (200 g, gradient: from DCM 100% up to DCM/MeOH 98/2) to afford Intermediate 10 (4.20 g, yield: 45%) as a yellowish oil.

### Intermediate 11

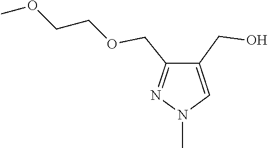

Lithium aluminum hydride (2 M in THF, 9.2 mL, 1 eq.) was added dropwise to a stirred solution of Intermediate 10 (4.2 g, 18.40 mmol) in dry THF (75 mL) at 0° C., under nitrogen atmosphere. The reaction mixture was stirred at 0° C. for 30 min. Wet THF (50 mL) was then added, followed by dropwise addition of water (5 mL). The reaction mixture was allowed to warm up to room temperature. Celite followed by MgSO₄ were added. The mixture was stirred at room temperature for 5 min. The suspension was filtered, the solid was washed with EtOAc, and the filtrate was concentrated under reduced pressure to afford Intermediate 11 (3.55 g, yield: 96%), used without further purification.

### Intermediate 12

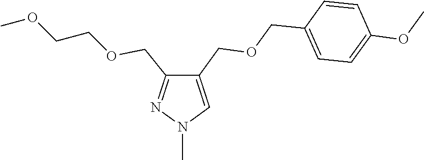

NaH (0.88 g, 1.25 eq.) was added portionwise to a solution of Intermediate 11 (3.55 g, 17.7 mmol) in dry DMF (50 mL) at 0° C., under nitrogen atmosphere. The reaction mixture was stirred at 0° C. for 10 min and was then allowed to warm up to room temperature for 20 min. 4-Methoxybenzyl chloride (3.12 mL, 1.3 eq.) and KI (0.29 g, 0.1 eq.) were added. The reaction mixture was stirred at room temperature for 16 h. The reaction was quenched with water (100 mL) and diluted with EtOAc (150 mL). The organic layer was separated and washed with brine (3×50 mL). The combined aqueous layer was back-extracted with EtOAc (100 mL). The combined organic layer was dried over MgSO₄, filtered, and evaporated. The residue was purified by flash column chromatography on silica gel (80 g, heptane/EtOAc 100/0 to 0/100) to afford Intermediate 12 (5.3 g, yield: 93%) as a colourless oil.

### Intermediate 13

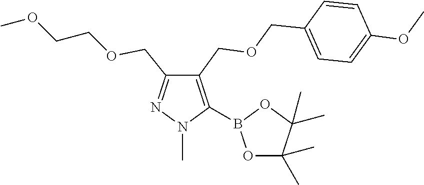

BuLi (2.5 M in hexane, 1.87 mL, 1.5 eq.) was added dropwise to a solution of Intermediate 12 (1.0 g, 3.12 mmol) in dry THF (16 mL) at −78° C., under nitrogen atmosphere. The mixture was stirred at −78° C. for 1 h, then 2-isopropoxy-4,4,5,5-tetramethyl-1,3,2-dioxaborolane (CAS [61676-62-8]) (1.0 mL, 1.60 eq.) was added dropwise. The reaction mixture was allowed to warm up to room temperature and was stirred for 2 h. The mixture was quenched by addition of saturated aqueous NH₄Cl (10 mL) and the mixture was diluted with EtOAc (50 mL) and water (5 mL). The organic layer was separated and washed with brine (10 mL), dried over MgSO₄, filtered, and concentrated under reduced pressure to afford Intermediate 13 (1.4 g, yield: 100%), used without further purification.

### Intermediate 14

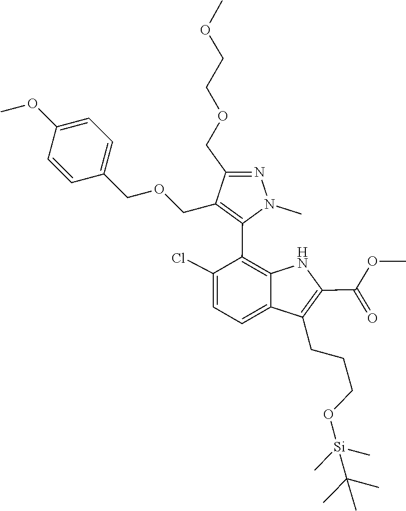

CPhos-Pd-G3 (CAS [1447963-73-6]) (79 mg, 0.05 eq.) was added to a mixture of Intermediate 13 (1.98 g, 2.2 eq.), Intermediate 1 (900 mg, 1.95 mmol) and K₂CO₃ (808 mg, 3.0 eq.) in 1,4-dioxane (20 mL) and water (2 mL), under nitrogen atmosphere. The reaction mixture was stirred at 40° C. for 3 h. The reaction mixture was diluted with EtOAc (100 mL) and washed with water (50 mL). The aqueous layer was extracted with EtOAc (50 mL), and the combined organic layer was dried over MgSO₄, filtered, and evaporated. The residue was purified by flash column chromatography on silica gel (120 g, gradient: DCM/MeOH 100/0 to 97/3) to afford Intermediate 14 (3.1 g), still impure and used without further purification.

### Intermediate 15

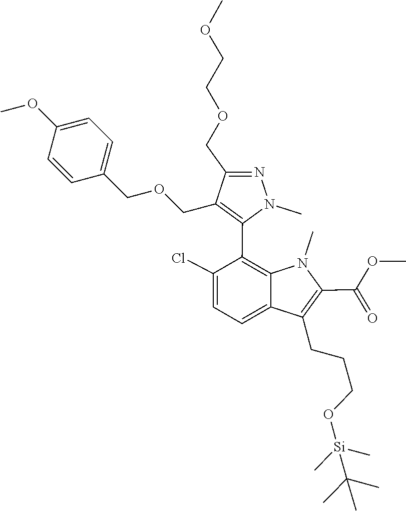

CH₃I (0.29 mL, 1.6 eq.) was added to a solution of Intermediate 14 (3.1 g, 2.92 mmol) and Cs₅CO₃ (1.42 g, 1.5 eq.) in DMF (10 mL). The reaction mixture was stirred at room temperature for 2 h. The mixture was diluted with EtOAc (75 mL) and water (50 mL). The organic layer was separated and washed with brine (2×25 mL). The aqueous layer was back-extracted with EtOAc (25 mL). The combined organic layer was dried over MgSO₄, filtered, and evaporated. The residue was purified by flash column chromatography on silica gel (80 g, gradient: heptane/EtOAc 100/0 to 0/100) to afford Intermediate 15 (820 mg, yield: 39%) as a colourless paste.

### Intermediate 16

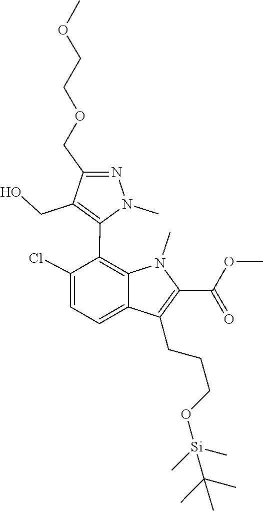 [SMILES: COCCOCc1nn(C)c(-c2c(Cl)ccc3c(CCCO[Si](C)(C)C(C)(C)C)c(C(=O)OC)n(C)c23)c1CO]

DDQ (343 mg, 1.5 eq.) was added to a solution of Intermediate 15 (720 mg, 1.0 mmol) in DCM (14 mL) and water (1.4 mL). The reaction mixture was stirred at room temperature for 2 h. The reaction mixture was diluted with DCM (50 mL) and treated with saturated aqueous NaHCO₃ (30 mL). The layers were separated, and the aqueous layer was back-extracted with DCM (30 mL). The combined organic layer was washed with brine (30 mL), dried over MgSO₄, filtered, and evaporated The residue was purified by flash column chromatography on silica gel (80 g, gradient: DCM/MeOH 100/0 to 97/3) to afford Intermediate 16 (587 mg, yield: 98%) as a colourless paste.

### Intermediate 17

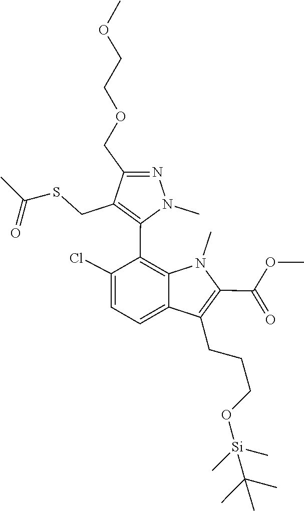 [SMILES: COCCOCc1nn(C)c(-c2c(Cl)ccc3c(CCCO[Si](C)(C)C(C)(C)C)c(C(=O)OC)n(C)c23)c1CSC(C)=O]

DIPEA (214.8 μL, 1.3 eq.) followed by MsCl (89 μL, 1.2 eq.) were added to a solution of Intermediate 16 (570 mg, 0.96 mmol) in dry DCM (11 mL) at 0° C. The reaction mixture was stirred at 0° C. for 30 min. A solution of potassium thioacetate (219 mg, 2 eq.) in dry DMF (6 mL) was then added and the reaction mixture was allowed to warm up to room temperature and was stirred for 16 h. The reaction mixture was diluted with EtOAc (50 mL) and water (30 mL). The layers were separated, and the aqueous layer was back-extracted with EtOAc (30 mL). The combined organic layer was washed with brine (3×20 mL), dried over MgSO₄, filtered, and evaporated. The residue was purified by flash column chromatography on silica gel (40 g, gradient: heptane/EtOAc 100/0 to 40/60) to afford Intermediate 17 (513 mg, yield: 82%) as a colourless paste.

### Intermediate 18

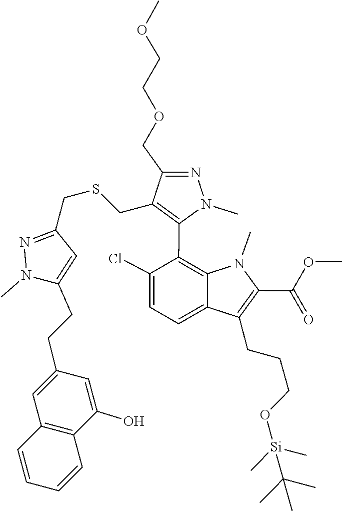

Intermediate 17 (505 mg, 0.77 mmol) and Intermediate 8 (501 mg, 1.2 eq.) were dissolved in MeOH (10 mL) and THF (3 mL). K₂CO₃ (214 mg, 2 eq.) was added and the reaction mixture was stirred at room temperature for 3 h. The mixture was concentrated under reduced pressure and the residue was partitioned between EtOAc (50 mL) and water (30 mL). The layers were separated, and the aqueous layer was back-extracted with EtOAc (2×30 mL). The combined organic layer was dried over MgSO₄, filtered, and evaporated. The residue was purified by flash column chromatography on silica gel (120 g, gradient: DCM/MeOH 100/0 to 94/6) to afford Intermediate 18 (508 mg, yield: 75%) as a foamy solid.

### Intermediate 19

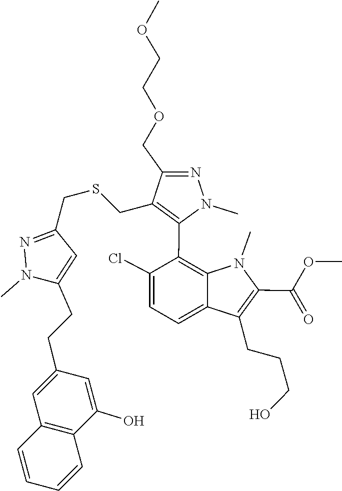

p-Toluenesulfonic acid monohydrate (146 mg; 1.1 eq.) was added to a solution of Intermediate 18 (610 mg, 0.69 mmol) in MeOH (6 mL). The reaction mixture was stirred at room temperature for 1 h. The reaction mixture was concentrated under reduced pressure and the residue was dissolved in DCM (30 mL) and washed with saturated aqueous NaHCO₃ (20 mL). The layers were separated, and the aqueous layer was back extracted with DCM (2×20 mL). The combined organic layer was washed with brine (30 mL), dried over MgSO₄, filtered, and evaporated. The residue was purified by flash column chromatography on silica gel (120 g, gradient: DCM/MeOH 100/0 to 95/5) to afford Intermediate 19 (465 mg, yield: 88%) as a foamy white solid.

### Intermediate 20 and Intermediate 21

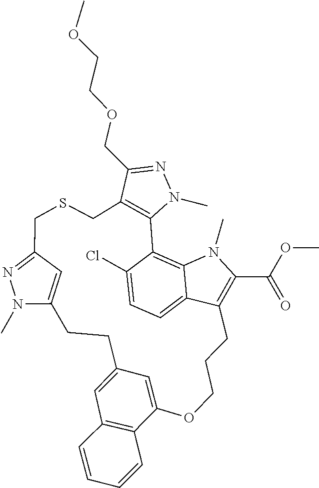 [0305] Intermediate 20: Rₐ or Sₐ; one atropisomer but absolute stereochemistry undetermined
Intermediate 21: Sₐ or Rₐ; one atropisomer but absolute stereochemistry undetermined

A solution of Intermediate 19 (465 mg, 0.611 mmol) and DTBAD (CAS [870-50-8]) (280 mg, 2.0 eq.) in toluene (11 mL) and THF (2 mL) was added with a syringe pump (0.1 mL/min) to a solution of PPh₃ (320 mg, 2 eq.) in toluene (11 mL), while stirring at 70° C. Once the addition was complete, the reaction mixture was allowed to cool to room temperature and was concentrated under reduced pressure. The residue was purified by flash column chromatography on silica gel (80 g, gradient: EtOAc/MeOH 100/0 to 99/1). The foamy white solid that was obtained was further purified by preparative SFC (Stationary phase: Daicel Chiralpak AD 20×250 mm, Mobile phase: CO₂, EtOH+0.4% iPrNH₂) to afford Intermediate 20 (155 mg, yield: 34%) and Intermediate 21 (160 mg, yield: 35%).

### Intermediate 22

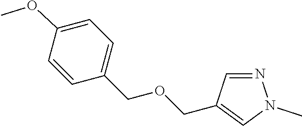

Sodium hydride (4.8 g, 1.2 eq) was added portionwise to a solution of 1-methyl-1H-pyrazol-4-yl)methanol (CAS [112029-98-8]) (11.2 g, 100 mmol) in DMF (220 mL) at 0° C. The reaction mixture was stirred at room temperature for 30 min. 4-Methoxybenzyl chloride (14.9 mL, 1.1 eq) and KI (1.6 g, 0.1 eq.) were then added. The reaction mixture was stirred at room temperature for 1 h. Water was added slowly (300 mL) and the mixture was extracted with EtOAc (300 mL×3). The combined organic layer was dried with Na₂SO₄, filtered, and evaporated. The residue was purified by flash column chromatography over silica gel (eluent: petroleum ether/EtOAc 100/0 to 0/100) to afford Intermediate 22 (16.42 g, yield: 71%) as a colorless oil.

### Intermediate 23

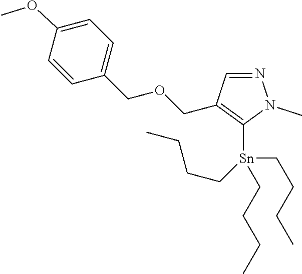

BuLi (2.5 M in hexanes, 42.4 mL, 1.5 eq) was added to a solution of Intermediate 22 (16.4 g, 70.6 mmol) in THE (320 mL) at −78° C. under nitrogen atmosphere. The reaction mixture was stirred at −78° C. for 1 h before chlorotributylstannane (34.5 g, 1.5 eq) was added slowly. The reaction mixture was stirred at room temperature for 3 h. The reaction mixture was poured slowly into saturated aqueous KF (300 mL) and the resulting mixture was stirred at room temperature overnight. The mixture was filtered. The filtrate was extracted with EtOAc (400 mL×3), washed with brine (600 mL), dried over Na₂SO₄, and evaporated to give a yellow oil. This oil was purified by flash column chromatography over silica gel (eluent: petroleum ether/EtOAc 100/0 to 0/100) to afford Intermediate 23 (29.42 g, yield: 80%) as a colorless oil.

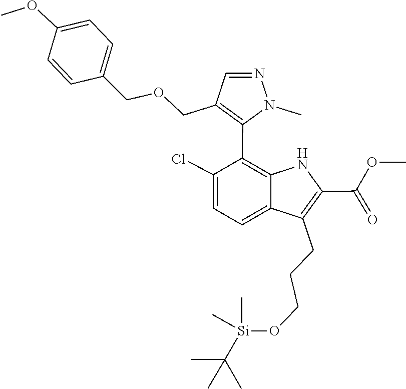

Intermediate 23 (3.38 g, 1.05 eq.) and K₃PO₄ (3.27 g, 2.5 eq.) were added to a stirred solution of Intermediate 1 (3.0 g, 6.18 mmol) in dry toluene (30 mL) under nitrogen atmosphere. Then, [1,1′-Bis(di-tert-butylphosphino)ferrocene]dichloropalladium (II) [95408-45-0] (409 mg, 0.1 eq.) was added and the reaction mixture was stirred at 125° C. for 16 h. Water (30 mL) was added and the mixture was extracted with EtOAc (2×50 mL). The layers were separated, washed with brine (30 mL), dried over Na₂SO₄, filtered, and evaporated. The residue was purified by flash column chromatography on silica gel (gradient: petroleum ether/EtOAc 100/0 to 40/60) to afford Intermediate 24 (2.96 g, yield: 78%) as a brown oil.

### Intermediate 25

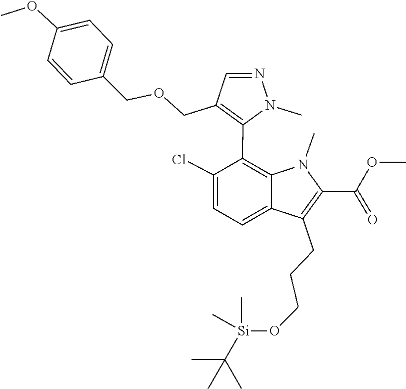

MeI (0.44 mL, 1.5 eq.) and Cs₂CO₃ (2.36 g, 1.5 eq.) were added to a stirred solution of Intermediate 24 (2.96 g, 4.83 mmol) in dry DMF (30 mL) under nitrogen atmosphere. The reaction mixture was stirred at room temperature for 16 h. Water (30 mL) was added and the mixture was extracted with EtOAc (2×50 mL). The organic layer was separated, washed with brine (30 mL), dried over Na₂SO₄, filtered, and concentrated under reduced pressure. The residue was purified by flash column chromatography on silica gel (gradient: petroleum ether/EtOAc 100/0 to 50/50) to afford Intermediate 25 (2.85 g, yield: 94%) as a yellow oil.

### Intermediate 26

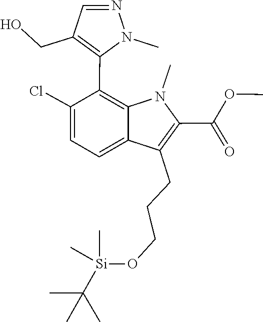 [SMILES: COC(=O)c1c(CCCO[Si](C)(C)C(C)(C)C)c2ccc(Cl)c(-c3c(CO)cnn3C)c2n1C]

DDQ (1.27 g, 1.5 eq.) was added to a stirred solution of Intermediate 25 (2.35 g, 3.75 mmol) in DCM (30 mL) and water (3 mL). The reaction mixture was stirred at room temperature for 2 h. Saturated aqueous NaHCO₃ (30 mL) was added and the mixture was extracted with DCM (2×50 mL). The organic layer was separated, washed with brine (30 mL), dried over Na₂SO₄, filtered, and evaporated at 35° C. to give a black oil that was purified by flash column chromatography on silica gel (gradient: petroleum ether/EtOAc 100/0 to 20/80) to afford Intermediate 26 (2.85 g, yield: 68%) as a pale yellow oil.

### Intermediate 27

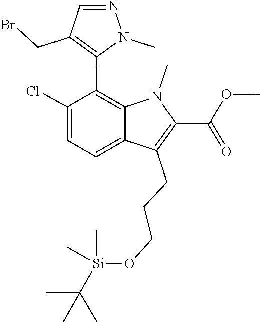 [SMILES: COC(=O)c1c(CCCO[Si](C)(C)C(C)(C)C)c2ccc(Cl)c(-c3c(CBr)cnn3C)c2n1C]

CBr₄ (668 mg, 2.0 eq.) was added in small portions to a stirred solution of Intermediate 26 (510 mg, 1 mmol) and PPh₃ (529 mg, 2 eq.) in DCM (10 mL) at 0° C. The mixture was stirred at 0° C. for 2 h. The solvent was concentrated in vacuo and the residue was purified by flash column chromatography on silica gel (gradient: petroleum ether/EtOAc 100/0 to 0/100) to afford Intermediate 27 (550 mg, yield: 95%) as a yellow solid.

### Intermediate 28

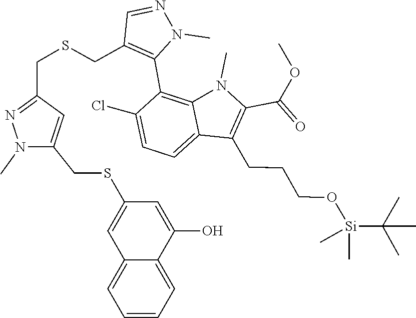 [SMILES: COC(=O)c1c(CCCO[Si](C)(C)C(C)(C)C)c2ccc(Cl)c(-c3c(CSCc4cc(CSc5cc(O)c6ccccc6c5)n(C)n4)cnn3C)c2n1C]

MeOH (10 mL) was added to a mixture of Intermediate 27 (550 mg, 0.96 mmol), Ethanethioic acid, S-[[5-[[[4-(acetyloxy)-2-naphthalenyl]thio]methyl]-1-methyl-1H-pyrazol-3-yl]methyl] ester (CAS [2245716-36-1]) (581 mg, 1.5 eq.) and PPh₃ (25 mg, 0.1 eq.). The resulting solution was cooled to 0° C. and degassed with nitrogen three times. K₂CO₃ (467 mg, 3.5 eq.) was added and the reaction mixture was stirred at room temperature for 2 h. The mixture was filtered and washed with EtOAc (20 mL). The filtrate was evaporated, and the residue was purified by flash column chromatography on silica gel (gradient: petroleum ether/EtOAc 100/0 to 0/100) to afford Intermediate 28 (420 mg, yield: 54%) as a yellow solid.

### Intermediate 29

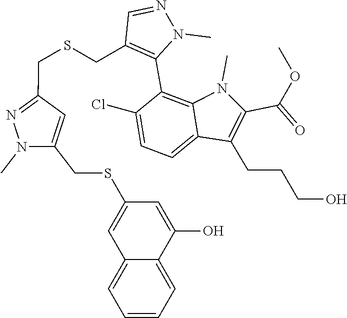 [SMILES: COC(=O)c1c(CCCO)c2ccc(Cl)c(-c3c(CSCc4cc(CSc5cc(O)c6ccccc6c5)n(C)n4)cnn3C)c2n1C]

A solution of Intermediate 28 (420 mg, 0.52 mmol), and TBAF (1 M in THF, 1 mL, 2 eq.) in THF (1 mL) was stirred at room temperature for 2 h. The reaction mixture was concentrated in vacuo and the residue was purified by flash column chromatography on silica gel (gradient: petroleum ether/EtOAc 100/0 to 0/100) to afford Intermediate 29 (265 mg, yield: 74%) as a yellow solid.

### Intermediate 30 and Intermediate 31

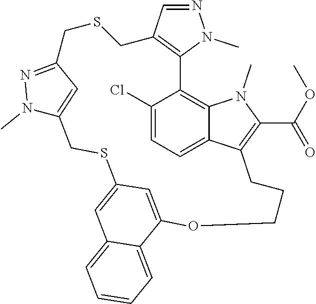 [SMILES: COC(=O)c1c2c3ccc(Cl)c(c3n1C)-c1c(cnn1C)CSCc1cc(n(C)n1)CSc1cc(c3ccccc3c1)OCCC2] [0324] Intermediate 30: Rₐ or Sₐ; one atropisomer but absolute stereochemistry undetermined
Intermediate 31: Sₐ or Rₐ; one atropisomer but absolute stereochemistry undetermined

PBu₃ (273 μL, 3.0 eq.) and ADD (CAS [10465-81-3]) (280 mg, 3.0 eq.) were added to a stirred solution of Intermediate 29 (255 mg, 0.369 mmol) in DCM (12 mL) under nitrogen atmosphere. The reaction mixture was stirred at room temperature for 16 h. Water (5 mL) and DCM (15 mL) were added. The organic layer was separated, dried over Na₂SO₄, filtered, and concentrated under reduced pressure. The residue was purified by flash column chromatography on silica gel (gradient: petroleum ether/EtOAc 100/0 to 0/100). The product obtained was further purified by preparative SFC (Stationary phase: Diacel Chiralpak AD (30×250 mm, 10 μm, Mobile phase: CO₂, 40% iPrOH+0.1% NH₃·H₂O) to afford Intermediate 30 (65 mg, yield: 47%) and Intermediate 31 (65 mg, yield: yield: 47%).

### Intermediate 32

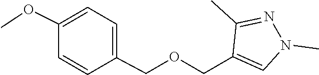

NaH (2.01 g, 1.2 eq.) was added to a solution of (1,3-dimethyl-1H-pyrazol-4-yl)methanol (CAS [103946-59-4]) (5.3 g, 42 mmol) in anhydrous DMF (100 mL) at 0° C. The reaction mixture was stirred at room temperature for 30 min. before p-methoxybenzyl chloride (7.24 g, 1.1 eq.) was added dropwise, followed by KI (697 mg, 0.1 eq.). The reaction mixture was stirred at room temperature for 1 h. The reaction was quenched by addition of saturated aqueous NH₄Cl (100 mL) and the mixture was extracted with EtOAc (150 mL×2). The combined organic layer was washed with brine (100 mL), dried over Na₂SO₄, and evaporated to give a yellow oil. This oil was purified by flash column chromatography over silica gel (eluent: petroleum ether/EtOAc 100/0 to 0/100) to afford Intermediate 32 (8.2 g, yield: 79%) as a yellow oil.

### Intermediate 33

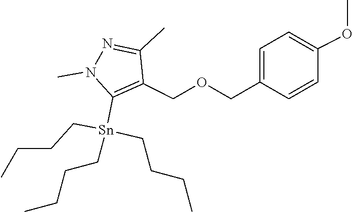 [SMILES: CCC[CH2][Sn]([CH2]CCC)([CH2]CCC)[c]1c(COCc2ccc(OC)cc2)c(C)nn1C]

Intermediate 33 was prepared according to an analogous procedure as for Intermediate 23, starting from Intermediate 32 instead of Intermediate 22.

### Intermediate 34

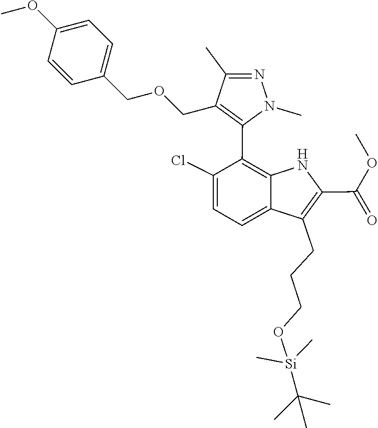 [SMILES: COC(=O)c1[nH]c2c(-c3c(COCc4ccc(OC)cc4)c(C)nn3C)c(Cl)ccc2c1CCCO[Si](C)(C)C(C)(C)C]

PdCl₂(dtbpf) (424 mg, 0.1 eq.) was added to a solution of Intermediate 1 (3 g, 6.5 mmol), Intermediate 33 (3.66 g, 1.05 eq.) and K₃PO₄ (3.45 g, 2.5 eq.) in toluene (30 mL) under nitrogen atmosphere and the reaction mixture was stirred at 125° C. overnight. Water (50 mL) was added and the mixture was extracted with EtOAc (100 mL×2). The combined organic layer was washed with brine (50 mL), dried over Na₂SO₄, and evaporated. The residue was purified by flash column chromatography over silica gel (eluent: petroleum ether/EtOAc 100/0 to 40/60) to afford Intermediate 34 (3 g, yield: 74%) as a brown oil.

### Intermediate 35

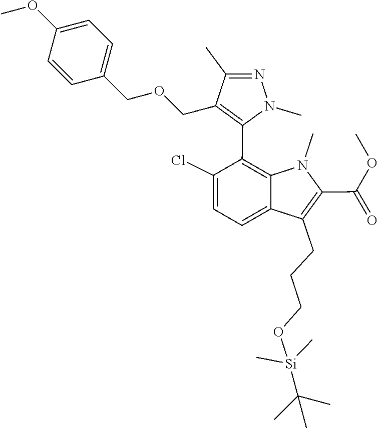 [SMILES: COC(=O)c1c(CCCO[Si](C)(C)C(C)(C)C)c2ccc(Cl)c(-c3c(COCc4ccc(OC)cc4)c(C)nn3C)c2n1C]

MeI (0.45 mL, 1.5 eq.) and Cs₂CO₃ (2.34 g, 1.5 eq.) were added to a solution of Intermediate 34 (3 g, 4.79 mmol) in anhydrous DMF (40 mL) and the reaction mixture was stirred at room temperature overnight. Water (60 mL) was added and the mixture was extracted with EtOAc (100 mL×2). The combined organic layer was washed with brine (50 mL), dried over Na₂SO₄, and evaporated to afford the Intermediate 35 (2.86 g, yield: 91%) as brown oil, used without further purification.

### Intermediate 36

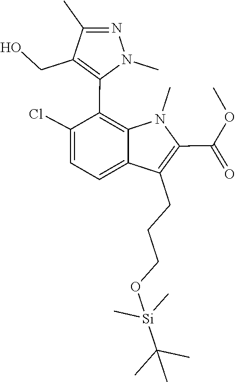 [SMILES: COC(=O)c1c(CCCO[Si](C)(C)C(C)(C)C)c2ccc(Cl)c(-c3c(CO)c(C)nn3C)c2n1C]

DDQ (1.49 g, 1.5 eq.), followed by water (4 mL) were added to a solution of Intermediate 35 in DCM (40 mL) and the mixture was stirred at room temperature for 2 h. The reaction was quenched by addition of saturated aqueous NaHCO₃ (30 mL) and the mixture was extracted with DCM (50 mL×2). The combined organic layer was washed with brine (30 mL), dried over Na₂SO₄, and evaporated at 35° C. The residue was purified by flash column chromatography over silica gel (eluent: petroleum ether/EtOAc 100/0 to 20/80) to afford Intermediate 36 (1.13 g, yield: 49%) as a yellow solid.

### Intermediate 37

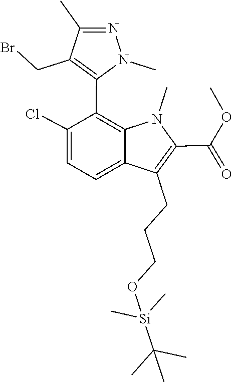 [SMILES: COC(=O)c1c(CCCO[Si](C)(C)C(C)(C)C)c2ccc(Cl)c(-c3c(CBr)c(C)nn3C)c2n1C]

CBr₄ (1.08 g, 1.5 eq.) and PPh₃ (0.85 g, 1.5 eq.) were added to a solution of Intermediate 36 (1.13 g, 2.17 mmol) in anhydrous DCM (20 mL) at 0° C. and the reaction mixture was stirred at the same temperature for 2 h. The solvent was evaporated and the residue was purified by flash column chromatography over silica gel (eluent: petroleum ether/EtOAc 100/0 to 70/30) to afford Intermediate 37 (630 mg, yield: 50%) as a colourless oil.

### Intermediate 38

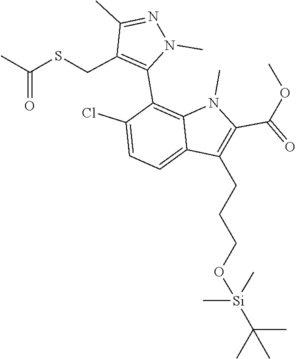 [SMILES: COC(=O)c1c(CCCO[Si](C)(C)C(C)(C)C)c2ccc(Cl)c(-c3c(CSC(C)=O)c(C)nn3C)c2n1C]

Potassium thioacetate (197 mg, 1.6 eq.) was added to a solution of Intermediate 37 (630 mg, 1.08 mmol) in ACN (12 mL) and the reaction mixture was stirred at room temperature for 1 h. Water (20 mL) was added and the mixture was extracted with EtOAc (20 mL×2). The combined organic layer was washed with brine (10 mL), dried over Na₂SO₄, and evaporated to afford Intermediate 38 (540 mg, yield: 78%) as a yellow oil, used without further purification.

### Intermediate 39

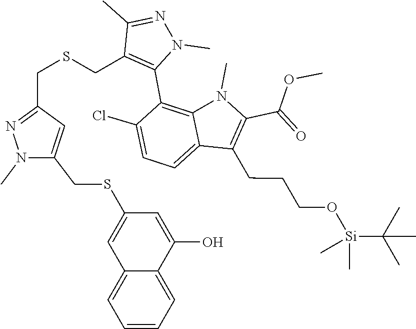 [SMILES: COC(=O)c1c(CCCO[Si](C)(C)C(C)(C)C)c2ccc(Cl)c(-c3c(CSCc4cc(CSc5cc(O)c6ccccc6c5)n(C)n4)c(C)nn3C)c2n1C]

3-[[[3-(Chloromethyl)-1-methyl-1H-pyrazol-5-yl]methyl]thio]-1-naphthalenol (CAS [2245716-35-0]) (218 mg, 1.5 eq.), PPh₃ (11 mg, 0.1 eq.), and K₂CO₃ (176 mg, 3 eq.) were added to a solution of Intermediate 38 (270 mg, 0.42 mmol) in anhydrous MeOH (5.4 mL). The reaction mixture was stirred at room temperature overnight. Water (10 mL) was added and the mixture was extracted with EtOAc (15 mL×2). The combined organic layer was washed with brine (10 mL), dried over Na₂SO₄, and evaporated to afford Intermediate 39 (490 mg, yield: 76%) as a brown oil, used without further purification.

### Intermediate 40

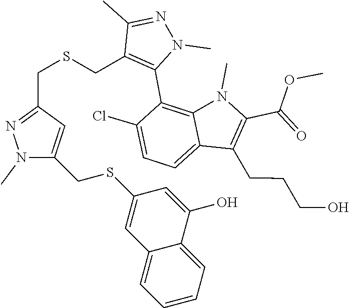 [SMILES: COC(=O)c1c(CCCO)c2ccc(Cl)c(-c3c(CSCc4cc(CSc5cc(O)c6ccccc6c5)n(C)n4)c(C)nn3C)c2n1C]

TBAF (1 M in TIE, 1.2 mL, 3.7 eq) was added to a solution of Intermediate 39 (490 mg, 0.32 mmol) in dry THE (5 mL) under nitrogen atmosphere and the reaction mixture was stirred at room temperature for 4 h. Water (10 mL) was added and the mixture was extracted with EtOAc (20 mL×2). The combined organic layer was washed with brine (10 mL), dried over Na₂SO₄, and evaporated. The residue was purified by flash column chromatography over silica gel (eluent: petroleum ether/EtOAc 100/0 to 0/100, then EtOAc/methanol 100/0 to 80/20) to afford Intermediate 40 (220 mg, yield: 89%) as a white solid.

### Intermediate 41 and Intermediate 42

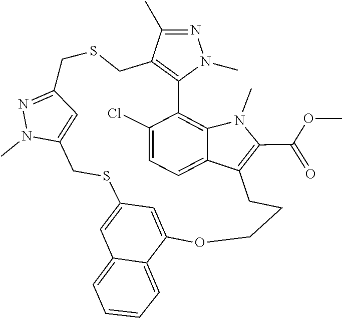 [0346] Intermediate 41: Rₐ or Sₐ; one atropisomer but absolute stereochemistry undetermined
Intermediate 42: Sₐ or Rₐ; one atropisomer but absolute stereochemistry undetermined

ADDP (CAS [10465-81-3]) (217 mg, 3 eq.) and n-Bu₃P (174 mg, 3 eq.) were added to a solution of Intermediate 40 (220 mg, 0.29 mmol) in anhydrous DCM (13 mL) and the reaction mixture was stirred at room temperature overnight. Water (10 mL) was added, the mixture was extracted with DCM (20 mL×2). The combined organic layers were combined, washed with brine (5 mL), dried over Na₂SO₄, and evaporated. The residue was purified by flash column chromatography over silica gel (eluent: petroleum ether/EtOAc 100/0 to 30/70). The obtained product was purified by preparative SFC (Stationary phase: Daicel Chiralpak AD (250 mm×30 mm, 10 um), Mobile phase: CO₂/iPrOH (0.1% NH₃·H₂O) 60/40) to afford Intermediate 41 (50 mg; yield: 36%) and Intermediate 42 (50 mg, yield: 36%), both as white solids.

### Intermediate 43

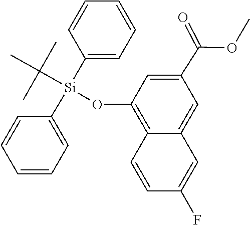 [SMILES: COC(=O)c1cc(O[Si](c2ccccc2)(c2ccccc2)C(C)(C)C)c2ccc(F)cc2c1]

TBDPSCl (14.66 g, 1.5 eq.) was added to a solution of methyl 7-fluoro-4-hydroxy-2-naphthoate (CAS [2092726-85-5]) (8 g, 35.555 mmol) and imidazole (7.26, 3 eq.) in DCM (500 mL), cooled to 0° C. under nitrogen atmosphere. Once the addition was complete, the reaction was stirred at room temperature overnight. The reaction was quenched by addition of water (100 mL). The mixture was extracted with EtOAc (3×200 mL). The combined organic layer was dried over Na₂SO₄, filtered, and concentrated to afford a yellow oil. This oil was purified by flash column chromatography on silica gel (petroleum ether:EtOAc—1:0 to 1:1) to afford Intermediate 43 (14 g, yield: 86%) as a yellow oils.

### Intermediate 44

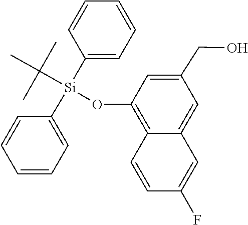 [SMILES: CC(C)(C)[Si](Oc1cc(CO)cc2cc(F)ccc12)(c1ccccc1)c1ccccc1]

LiAlH₄ (1.39 g, 1.2 eq.) was added slowly to a solution of Intermediate 43 (14 g, 30.528 mmol) in THF (200 mL), cooled to 0° C. under nitrogen atmosphere. Once the addition was complete the reaction mixture was stirred at 0° C. for 2 h. The reaction was quenched by slow addition of water (2 mL) followed by a 10% aqueous NaOH solution (2 mL) at 0° C. The heterogeneous mixture was filtered, and the filter cake was washed with DCM (200 mL). The filtrate was evaporated and the residue was purified by flash column chromatography on silica gel (petroleum ether:EtOAc—1:0 to 1:1) to give Intermediate 44 (12 g, yield: 90%) as a yellow solid.

### Intermediate 45

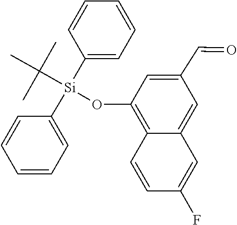 [SMILES: CC(C)(C)[Si](Oc1cc(C=O)cc2cc(F)ccc12)(c1ccccc1)c1ccccc1]

MnO₂ (29.074 g, 12 eq.) was added to a solution of Intermediate 44 (12 g, 27.869 mmol) in DCM (200 mL) at room temperature. The resulting solution was stirred at room temperature overnight. The reaction mixture was filtered and the filtrate was concentrated. The residue was purified by flash column chromatography over silica gel (eluent: petroleum ether/EtOAc, 100/0 to 50/50) to afford Intermediate 45 (12 g, yield: 99%) as a yellow oil.

### Intermediate 46

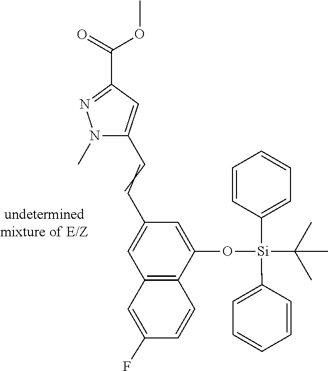 [SMILES: COC(=O)c1cc(C=Cc2cc(O[Si](c3ccccc3)(c3ccccc3)C(C)(C)C)c3ccc(F)cc3c2)n(C)n1]

NaH (60% in mineral oil, 1.448 g, 1.3 eq.) was added to a suspension of Intermediate 73 (13.812 g, 1.1 eq.) in THF (200 mL) at 0° C. The resulting solution was stirred at this temperature for 1 h before being cooled to −30° C. Intermediate 45 (12 g, 27.847 mmol) was added slowly to the solution while maintaining the temperature between −20° C. and −30° C. Once the addition was complete, the reaction was stirred at −30° C. for 2 h. The reaction was quenched by slow addition of water (100 mL). The mixture was extracted with DCM (3×300 mL). The combined organic layer was dried over Na₂SO₄, filtered, and concentrated under reduced pressure. The crude product was purified by column chromatography on silica gel (petroleum ether:EtOAc—1:0 to 1:1) to afford Intermediate 46 (13 g, yield: 82%) as a white solid.

### Intermediate 47

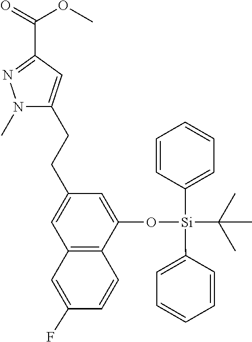 [SMILES: COC(=O)c1cc(CCc2cc(O[Si](c3ccccc3)(c3ccccc3)C(C)(C)C)c3ccc(F)cc3c2)n(C)n1]

A solution of Intermediate 46 (13 g, 23.02 mmol) in MeOH (75 mL) and THF (75 mL) was hydrogenated at 25° C. (15 psi 112) in the presence of Pd/C (10%, 2 g). The reaction mixture was stirred for 16 h. After uptake of H₂ (1 eq.), the catalyst was filtered off and the filtrate was evaporated to afford Intermediate 47 (13 g, yield: 100%) as a colorless oil.

### Intermediate 48

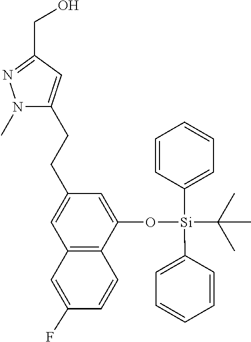 [SMILES: Cn1nc(CO)cc1CCc1cc(O[Si](c2ccccc2)(c2ccccc2)C(C)(C)C)c2ccc(F)cc2c1]

LiAlH₄ (1.045 g, 1.2 eq.) was added portionwise to a solution of Intermediate 47 (13 g, 22.94 mmol) in THE (200 mL) at 0° C., under nitrogen atmosphere. The reaction mixture was stirred at 0° C. for 2 h. Water (1 mL) was then added dropwise, followed by a 10% aqueous NaOH solution (1 mL), at 0° C. The reaction mixture was filtered, the filter cake was washed with DCM (200 mL), and the filtrate was evaporated. The crude product was purified by flash column chromatography over silica gel (eluent: petroleum ether/EtOAc, 100/0 to 0/100) to afford Intermediate 48 (10.4 g, yield: 84%) as a white solid.

### Intermediate 49

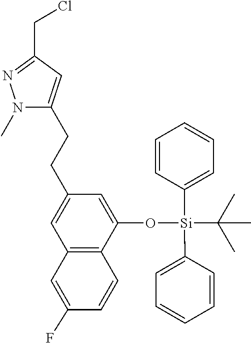 [SMILES: Cn1nc(CCl)cc1CCc1cc(O[Si](c2ccccc2)(c2ccccc2)C(C)(C)C)c2ccc(F)cc2c1]

A solution of Intermediate 48 (12 g, 22.27 mmol) in dry DCM (140 mL) was cooled to 0° C. under nitrogen atmosphere. SOCl₂ (1.86 mL, 1.15 eq.) was added dropwise. The reaction mixture was allowed to warm up to room temperature and was stirred for 30 min. The reaction mixture was diluted with DCM (100 mL) and saturated aqueous NaHCO₃ (100 mL). The aqueous layer was separated and the organic one was washed with saturated aqueous NaHCO₃ (25 mL) and brine (25 mL). The combined organic layer was dried over MgSO₄, filtered, and concentrated under reduced pressure to give Intermediate 49 (12.4 g, considered quantitative) as a colorless oil that solidified on standing.

### Intermediate 50

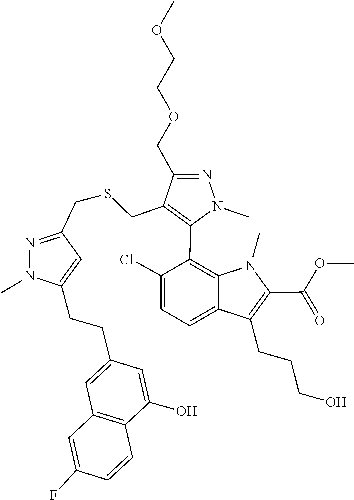

Intermediate 17 (115 mg, 0.176 mmol) and Intermediate 49 (118 mg, 1.2 eq.) were dissolved in a mixture of MeOH (2.3 mL) and THF (0.7 mL). K₂CO₃ (49 mg, 2 eq.) was then added and the reaction mixture was stirred at room temperature for 48 h. The reaction mixture was concentrated under reduced pressure and the residue was partitioned between water (20 mL) and EtOAc (30 mL). The layers were separated and the aqueous layer was extracted with EtOAc (2×15 mL). The combined organic layer was dried over MgSO₄, filtered, and concentrated under reduced pressure. The residue was dissolved in MeOH (5 mL) and pTsOH (101 mg, 3 eq.) was added. The reaction mixture was stirred at room temperature for 1 h. The reaction mixture was concentrated under reduced pressure. The residue was dissolved in DCM (20 mL) and washed with saturated aqueous NaHCO₃ (10 mL). The aqueous layer was extracted with DCM (2×10 mL) and the combined organic layer was dried over MgSO₄, filtered, and concentrated under reduced pressure. The residue was purified by flash column chromatography on silica gel (40 g, gradient: DCM/MeOH 100/0 to 96/4) to afford Intermediate 50 (95 mg, yield: 69%) as a foam.

### Intermediate 51 and Intermediate 52

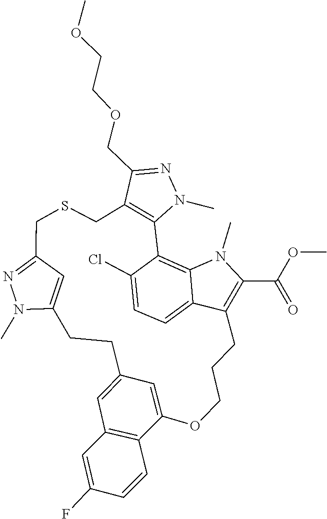 [0366] Intermediate 51: Sₐ or Rₐ; one atropisomer but absolute stereochemistry undetermined
Intermediate 52: Rₐ or Sₐ; one atropisomer but absolute stereochemistry undetermined

A solution of Intermediate 50 (95 mg, 0.122 mmol) and DTBAD (80 mg, 2.5 eq.) in toluene (2.5 mL) and THF (0.5 mL) was added with a syringe pump (0.1 mL/min) to a solution of PPh₃ (80 mg, 2.5 eq.) in toluene (2.5 mL) stirring at 70° C. Once the addition was complete, the reaction mixture was cooled to room temperature and the solvents were evaporated. The residue was purified by flash column chromatography on silica gel (24 g, gradient: EtOAc/MeOH 100/0 to 99/1) to afford a white solid. This solid was separated atropisomers by preparative SFC (Stationary phase: Chiralpak Daicel IC 20×250 mm, Mobile phase: CO₂, EtOH+0.4% iPrNH₂) to afford Intermediate 51 (21 mg, yield: 23%) and Intermediate 52 (23 mg, yield: 25%).

### Intermediate 53

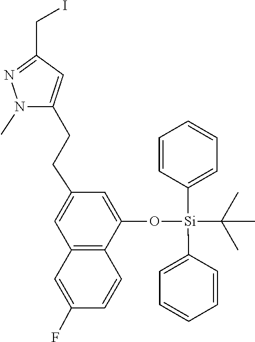 [SMILES: Cn1nc(CI)cc1CCc1cc(O[Si](c2ccccc2)(c2ccccc2)C(C)(C)C)c2ccc(F)cc2c1]

DIPEA (0.64 mL, 2 eq.) followed by methanesulfonic anhydride (0.65 g, 2 eq.) was added to a solution of Intermediate 48 (1.0 g, 1.86 mmol) in THF (45 mL), cooled to 0° C. The reaction mixture was stirred at room temperature for 0.5 h. Sodium iodide (1.39 g, 5 eq.) was then added to the mixture and it was further stirred at room temperature for 1 h. The reaction mixture was diluted with DCM (100 mL) and washed with water (20 mL). The aqueous layer was extracted with DCM/iPrOH 3:1 (2×30 mL), the combined organic layer was dried over MgSO₄, and concentrated under reduced pressure to give a dark yellow oil. This oil was purified by flash column chromatography on silica gel (SiO₂, 24 g column, 0-3% MeOH in DCM) to give Intermediate 53 (1.1 g, yield: 91%)

### Intermediate 54

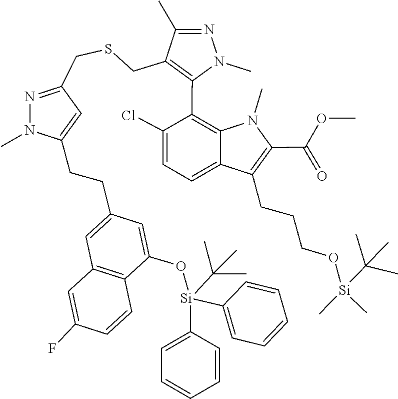 [SMILES: COC(=O)c1c(CCCO[Si](C)(C)C(C)(C)C)c2ccc(Cl)c(-c3c(CSCc4cc(CCc5cc(O[Si](c6ccccc6)(c6ccccc6)C(C)(C)C)c6ccc(F)cc6c5)n(C)n4)c(C)nn3C)c2n1C]

MeOH (10 mL) was added to a mixture of Intermediate 53 (450 mg, 0.694 mmol), Intermediate 38 (441 mg, 1.1 eq.), and PPh₃ (18 mg, 0.1 eq.). The resulting solution was cooled to 0° C. and degassed with nitrogen three times. K₂CO₃ (288 mg, 3 eq.) was added and the reaction mixture was stirred at room temperature overnight. The reaction mixture was filtered and the filtrate was evaporated to give Intermediate 54 (680 mg, yield: 93%), used without further purification.

### Intermediate 55

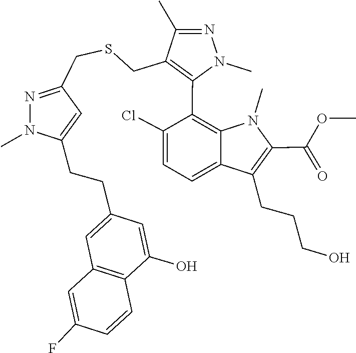

TBAF (1 M solution in THF, 1.96 mL, 3 eq.) was added to a solution of Intermediate 54 (680 mg, 0.653 mmol) in TIE (1 mL) at 0° C. The reaction mixture was stirred at room temperature for 2 h. Water (1 mL) was added dropwise and the mixture was filtered. The layers were separated and the organic layer was evaporated under pressure. The residue was purified by column chromatography over silica gel (eluent: petroleum ether/EtOAc 100/0 to 1/2) to afford Intermediate 55 (400 mg, yield: 87%) as a white solid.

### Intermediate 56 and Intermediate 57

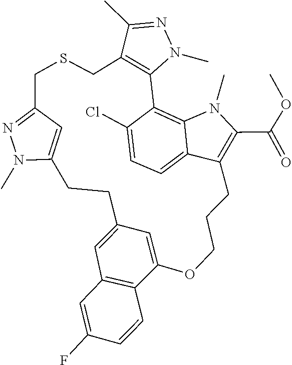 [SMILES: COC(=O)c1c2c3ccc(Cl)c(c3n1C)-c1c(c(C)nn1C)CSCc1cc(n(C)n1)CCc1cc(c3ccc(F)cc3c1)OCCC2] [0376] Intermediate 56: Rₐ or Sₐ; one atropisomer but absolute stereochemistry undetermined
Intermediate 57: Sₐ or Rₐ; one atropisomer but absolute stereochemistry undetermined

n-Bu₃P (319 mg, 3 eq.) and ADDP (CAS [10465-81-3]) (398 mg, 3 eq.) were added to a solution of Intermediate 55 (370 mg, 0.525 mmol) in DCM (2 mL) at room temperature under nitrogen atmosphere. The reaction mixture was stirred at room temperature overnight. Water (10 mL) was added to the reaction mixture. The layers were separated and the aqueous layer was extracted with DCM (10 mL×3). The combined organic layer was dried with Na₂SO₄, filtered, and evaporated. The residue was purified by flash column chromatography over silica gel (eluent: petroleum ether/EtOAc 100/0 to 0/100). The obtained product was purified by preparative SFC (Column: Phenomenex-Cellulose-2 (250 mm×30 mm, 5 μm), solvent A: Supercritical CO₂, solvent B: 0.1% NH₃ MeOH, A/B 45/55) to afford Intermediate 56 (108 mg, yield: 30%) and Intermediate 57 (112 mg, yield: 31%), both as yellow solids.

### Intermediate 58

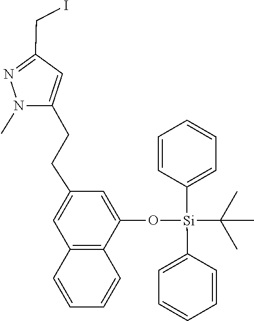 [SMILES: Cn1nc(CI)cc1CCc1cc(O[Si](c2ccccc2)(c2ccccc2)C(C)(C)C)c2ccccc2c1]

Ms₂O (468 mg, 2 eq.) was added slowly to the mixture of Intermediate 7 (700 mg, 1.344 mmol) and DIPEA (667 μL, 3 eq.) in DCM (15 mL) at 0° C. The reaction mixture was stirred at room temperature for 5 h. LiI (540 mg, 3 eq.) was then added to the reaction mixture at 0° C. and the reaction mixture was stirred at room temperature for 16 h. The reaction was quenched by addition of water (20 mL) and the mixture was extracted with DCM (30 mL×2). The organic layers was concentrated in vacuum to give Intermediate 58 (1.2 g, impure), used immediately in the next step.

### Intermediate 59

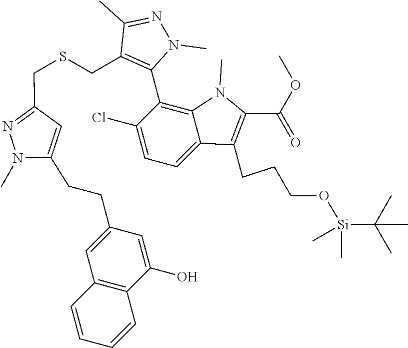 [SMILES: COC(=O)c1c(CCCO[Si](C)(C)C(C)(C)C)c2ccc(Cl)c(-c3c(CSCc4cc(CCc5cc(O)c6ccccc6c5)n(C)n4)c(C)nn3C)c2n1C]

K₂CO₃ (489 mg, 3 eq.) and PPh₃ (31 mg, 0.1 eq.) were added to a solution of Intermediate 58 (1.2 g, 1.18 mmol) and Intermediate 38 (598 mg, 0.8 eq.) in MeOH. The reaction mixture was stirred at room temperature for 16 h. The residue was partitioned between water (50 mL) and EtOAc (50 mL). The layers were separated and the aqueous layer was extracted with EtOAc (50 mL). The combined organic layer was dried with Na₂SO₄, filtered, and evaporated. The residue was purified by flash column chromatography over silica gel (petroleum ether/EtOAc 100/0 to 20/80) to afford Intermediate 59 (690 mg, 75% pure, yield: 55%) as a white solid.

### Intermediate 60

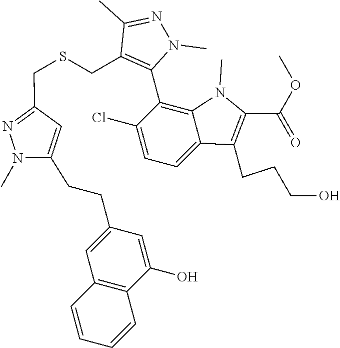 [SMILES: COC(=O)c1c(CCCO)c2ccc(Cl)c(-c3c(CSCc4cc(CCc5cc(O)c6ccccc6c5)n(C)n4)c(C)nn3C)c2n1C]

TBAF (1 M in THF, 1.3 mL, 2 eq.) was added to a solution of Intermediate 59 (690 mg, 0.653 mmol) in THF (8 mL). The reaction mixture was stirred at room temperature for 16 h. EtOAc (60 mL) was added to the reaction mixture and it was washed with water (20 mL×2) and brine (20 mL), dried with Na₂SO₄, filtered, and evaporated. The residue was purified by flash column chromatography over silica gel (petroleum ether/EtOAc 100/0 to 0/100) to afford Intermediate 60 (450 mg, yield: 99%) as a white solid.

### Intermediate 61 and Intermediate 62

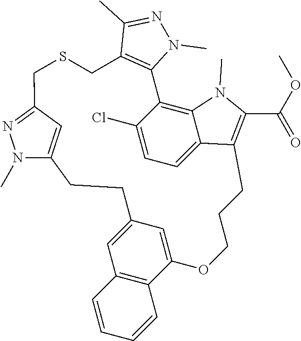 [SMILES: COC(=O)c1c2c3ccc(Cl)c(c3n1C)-c1c(c(C)nn1C)CSCc1cc(n(C)n1)CCc1cc(c3ccccc3c1)OCCC2] [0386] Intermediate 61: Rₐ or Sₐ; one atropisomer but absolute stereochemistry undetermined
Intermediate 62: Sₐ or Rₐ; one atropisomer but absolute stereochemistry undetermined

ADDP (CAS [10465-81-3]) (489 mg, 3 eq.) was added to a solution of Intermediate 60 (450 mg, 0.645 mmol) and n-Bu₃P (479 μL, 3 eq.) in DCM (40 mL) at 0° C. under nitrogen atmosphere. The reaction mixture was stirred at room temperature for 16 h. The reaction mixture was concentrated in vacuum and the residue was purified by flash column chromatography over silica gel (petroleum ether/EtOAc 100/0 to 10/90) to afford a white solid. This solid was separated into atropisomers by preparative SFC (Daicel Chiralpak IC (250 mm×30 mm, 5 um); Mobile phase: A: Supercritical CO₂, B: 0.1% NH₃·H₂O in EtOH, A:B=60:40) to afford Intermediate 61 (140 mg, yield: 29%) and Intermediate 62 (120 mg, yield: 27%), both as white solids.

### Intermediate 63

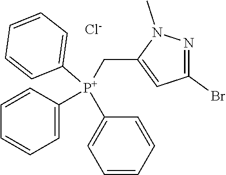 [SMILES: Cn1nc(Br)cc1C[P+](c1ccccc1)(c1ccccc1)c1ccccc1 ; [Cl-]]

PPh₃ (19.53 g, 1.2 eq.) was added to a solution of 3-bromo-5-(chloromethyl)-1-methyl-1H-pyrazole (CAS [2109428-60-4]) (13 g, 62 mmol) in ACN (150 mL) and the reaction mixture was stirred at 85° C. for 16 h. The solvent was evaporated. The residue was added to petroleum ether (100 mL) and this mixture was stirred at room temperature for 1 h. The solid was filtered and dried under vacuum to give Intermediate 63 (25 g, yield: 84%) as a white solid.

### Intermediate 64

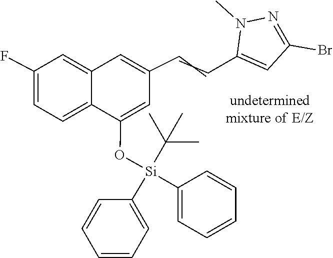 [SMILES: Cn1nc(Br)cc1C=Cc1cc(O[Si](c2ccccc2)(c2ccccc2)C(C)(C)C)c2ccc(F)cc2c1]

NaH (60% in mineral oil, 975 mg, 1.3 eq.) was added to a solution of Intermediate 63 (9 g, 18.76 mmol) in THF (100 mL) at 0° C. and the reaction mixture was stirred at 0° C. for 1 h. Intermediate 45 (9.79 g, 1.1 eq.) was then added at −30° C. and the reaction mixture was stirred at −30° C. for 2 h. The reaction was quenched by addition of aqueous NH₄Cl (50 mL). The mixture was extracted with EtOAc (100 mL×3). The combined organic layer was dried with Na₂SO₄ and the solvent was evaporated to give the crude product as a yellow oil. This oil was purified by column chromatography on silica gel (eluent: petroleum ether/EtOAc 100/0 to 85/15) to give Intermediate 64 (9 g, yield: 81%) as a white solid.

### Intermediate 65

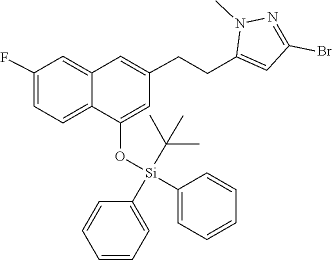 [SMILES: Cn1nc(Br)cc1CCc1cc(O[Si](c2ccccc2)(c2ccccc2)C(C)(C)C)c2ccc(F)cc2c1]

PtO₂ (1.04 g, 0.3 eq.) was added to a solution of Intermediate 64 (9 g, 15.25 mmol) in EtOAc (100 mL) under hydrogen atmosphere. The reaction mixture was stirred at room temperature for 6 h. The reaction mixture was filtered and the filtrate was evaporated. The residue was purified by column chromatography on silica gel (eluent: petroleum ether/EtOAc 100/0 to 85/15) to give Intermediate 65 (7.4 g, yield: 83%) as a white solid.

### Intermediate 66

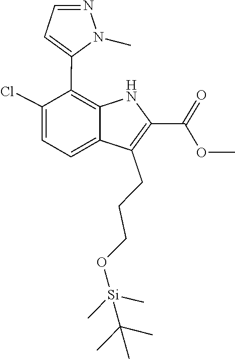 [SMILES: COC(=O)c1[nH]c2c(-c3ccnn3C)c(Cl)ccc2c1CCCO[Si](C)(C)C(C)(C)C]

Intermediate 1 (1 g, 2.17 mmol) was dissolved in 1,4-dioxane (28 mL). 1-Methyl-1H-pyrazole-5-boronic acid pinacol ester (CAS [847818-74-0]) (542 mg, 1.2 eq.), K₂CO₃ (600 mg, 2 eq.) and bis(di-tert-butyl(4-dimethylaminophenyl)phosphine)dichloropalladium(II) (CAS [887919-35-9]) (154 mg, 0.1 eq.), followed by water (2 mL) were added. The reaction mixture was stirred at 90° C. for 2 h. To push the reaction to completion, more boronate (452 mg, 1 eq.) and Pd catalyst (154 mg, 0.1 eq.) were added after 2 h. After 16 h of reaction, the reaction mixture was filtered over dicalite and concentrated in vacuo. The crude mixture was poured into water and the resulting solid was filtered and dried under vacuum to give Intermediate 66 (880 mg, yield: 81%) as a light yellow solid.

### Intermediate 67

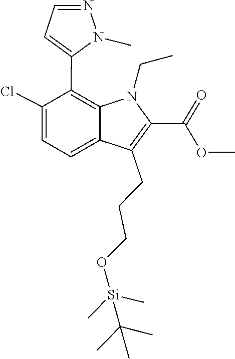 [SMILES: CCn1c(C(=O)OC)c(CCCO[Si](C)(C)C(C)(C)C)c2ccc(Cl)c(-c3ccnn3C)c21]

Intermediate 66 (880 mg, 1.75 mmol) was dissolved in DMF (14 mL). Cs₂CO₃ (1142 mg, 2 eq.) and iodoethane (0.28 mL, 2 eq.) were added and the reaction mixture was stirred for 1 h at 80° C. The mixture was partitioned between EtOAc and water and the layers were separated. The combined organic layer was dried with MgSO₄, filtered, and evaporated. The residue was purified by column chromatography on silica gel (heptane/EtOAc 100/0 to 10/90) to give Intermediate 67 (850 mg, 99%).

### Intermediate 68

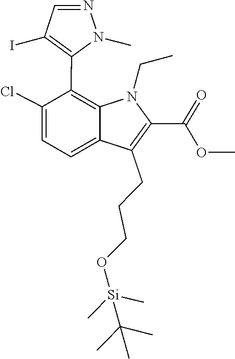 [SMILES: CCn1c(C(=O)OC)c(CCCO[Si](C)(C)C(C)(C)C)c2ccc(Cl)c(-c3c(I)cnn3C)c21]

Intermediate 67 (3 g, 5.2 mmol) was dissolved in dry DCM (33 mL). N-iodosucccinimide (1405 mg, 1.2 eq.) and zinc bis(trifluoromethylsulfonyl)imide (CAS [168106-25-0]) (977 mg, 0.3 eq.) were added. The reaction mixture was stirred at 50° C. for 16 h. The solution was concentrated in vacuo and directly purified by column chromatography on silica gel (heptane/EtOAc: 100/0 to 10/90) to give Intermediate 68 (1.42 g, yield: 44%). Intermediate 69

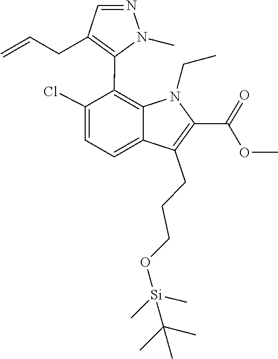 [SMILES: C=CCc1cnn(C)c1-c1c(Cl)ccc2c(CCCO[Si](C)(C)C(C)(C)C)c(C(=O)OC)n(CC)c12]

Intermediate 68 (881 mg, 1.43 mmol) was dissolved in dry DMF (11 mL). LiCi (67 mg, 1.1 eq.), allyltributyltin (0.9 mL, 2 eq.), and Pd(PPh₃)₄ (83 mg, 0.05 eq.) were added and the vial was sealed. The reaction mixture was stirred at 85° C. in a microwave oven for 4 h. The reaction mixture was partitioned between EtOAc and water. The layers were separated and the organic layer was washed with water. The combined organic layer was dried with MgSO₄, filtered, and concentrated in vacuo. The residue was purified by preparative HPLC (Stationary phase: RP XBridge Prep C18 OBD—5 μm, 50×250 mm, Mobile phase: 0.25% NH₄HCO₃ solution in water, CH₃CN) to give Intermediate 69 (483 mg, yield: 64%).

### Intermediate 70

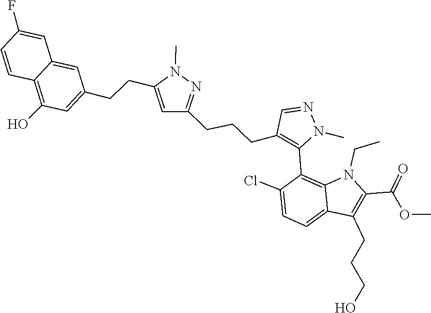 [SMILES: CCn1c(C(=O)OC)c(CCCO)c2ccc(Cl)c(-c3c(CCCc4cc(CCc5cc(O)c6ccc(F)cc6c5)n(C)n4)cnn3C)c21]

Intermediate 69 (741 mg, 1.4 mmol) was dissolved in THE (16 mL) and 9-BBN (CAS [280-64-8]) (14 mL, 0.5 M in THF, 5 eq.) was added. The reaction mixture was stirred at 75° C. for 40 min. The solution was cooled to room temperature and Intermediate 65 (821 mg, 1 eq.), K₃PO₄ (890 mg, 3 eq.), and Pd(dtbpf)Cl₂ (CAS [95408-45-0]) (182 mg, 0.2 eq.) were added in one portion to the solution. The solution was degassed with nitrogen and the vial was sealed. Water (1.5 mL) was then added and the reaction mixture was stirred at 80° C. for 1 h. The reaction mixture was concentrated in vacuo and the residue was directly purified by column chromatography (heptane/EtOAc 100/0 to 10/90) to give a light yellow oil. This oil was dissolved in MeOH (20 mL) and p-toluene sulfonic acid monohydrate (1329 mg, 5 eq.) was added. The reaction mixture was stirred at room temperature for 1 h. The solution was concentrated in vacuo and diluted with EtOAc. This solution was washed with a saturated aqueous NaHCO₃ solution. The organic layer was dried with MgSO₄, filtered, and concentrated in vacuo. The residue was purified by column chromatography on silica gel (DCM/MeOH 100/0 to 90/10) to give Intermediate 70 (400 mg, yield: 42%) as a light yellow oil.

### Intermediate 71 and Intermediate 72

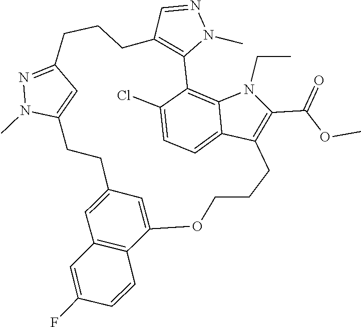 [0405] Intermediate 71: Sₐ or Rₐ; one atropisomer but absolute stereochemistry undetermined
Intermediate 72: Rₐ or Sₐ; one atropisomer but absolute stereochemistry undetermined

PPh₃ (573 mg, 4 eq.) was dissolved in toluene (17 mL), previously degassed with nitrogen. This solution (Solution A) was thoroughly degassed with nitrogen for 15 min. In another vial, Intermediate 70 (375 mg, 0.55 mmol) was dissolved in previously degassed toluene (17 mL) and THE (3 mL). This solution (Solution B) was thoroughly degassed with nitrogen for 10 min. Solution B was then added dropwise by syringe pump (0.1 mL/min) to Solution A, warmed to 70° C. After the addition was complete, the reaction mixture was further stirred at 70° C. for 10 min before it was concentrated in vacuo. The residue was purified by preparative HPLC (Stationary phase: RP XBridge Prep C18 OBD—10 μm, 50×150 mm, Mobile phase: 0.25% NH₄HCO₃ solution in water, CH₃CN) followed by preparative SFC (Stationary phase: Chiralpak Diacel AD 20×250 mm, Mobile phase: CO₂, EtOH+0.4% iPrNH₂) to afford Intermediate 71 (50 mg, yield: 14%) and Intermediate 72 (30 mg, yield: 8%).

### Intermediate 73

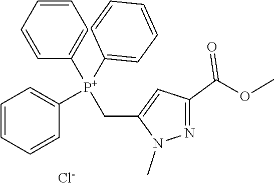 [SMILES: COC(=O)c1cc(C[P+](c2ccccc2)(c2ccccc2)c2ccccc2)n(C)n1 ; [Cl-]]

A solution of 5-(chloromethyl)-1-methyl-1H-pyrazole-3-carboxylic acid methyl ester (CAS [2245938-86-5], 24 g, 0.13 mol) and triphenylphosphine (37 g, 0.13 mol, 1.05 eq.) in ACN (250 mL) was refluxed for 16 h. The white suspension was concentrated in vacuo and triturated with EtOAc (100 mL) for 16 h. The solid was collected by filtration and dried to afford Intermediate 73 (54.8 g, yield 96%) as a white solid

### Intermediate 74

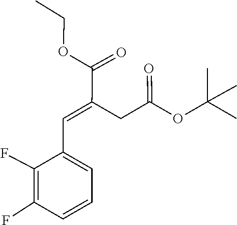 [SMILES: CCOC(=O)/C(=C/c1cccc(F)c1F)CC(=O)OC(C)(C)C]

NaH (60% in mineral oil, 61.9 g, 1548.2 mmol, 1.1 eq.) was added to a solution of 4-(tert-butyl) 1-ethyl 2-(diethoxyphosphoryl)succinate (CAS [77924-28-8], 523.8 g, 1548.2 mmol, 1.1 eq.) in THE (3500 mL) at 0° C. The resulting solution was stirred at 0° C. for 1 h. Then, 2,3-difluorobenzaldehyde (200 g, 1407.4 mmol), dissolved in THE (1500 mL), was added to the solution and the reaction mixture was stirred at room temperature for 3 h. The reaction was quenched by addition of cold water (2000 mL). The resulting mixture was extracted with EtOAc (3×3000 mL). The combined organic layer was dried over Na₂SO₄, filtered, and concentrated to afford Intermediate 74 (538 g, assumed quantitative) as a yellow oil, used without further purification.

### Intermediate 75

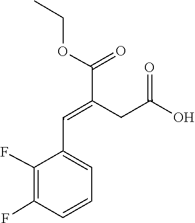 [SMILES: CCOC(=O)/C(=C/c1cccc(F)c1F)CC(=O)O]

Intermediate 74 (538 g, 1648.6 mmol) was dissolved in TFA (2000 mL) and the reaction mixture was stirred at room temperature for 1 h. The reaction mixture was concentrated under reduced pressure. Toluene was added and evaporated under reduced pressure to afford Intermediate 75 (533 g, assumed quantitative) as a yellow solid, used without further purification.

### Intermediate 76

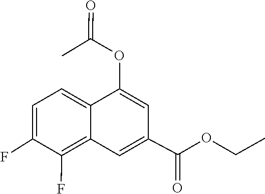 [SMILES: CCOC(=O)c1cc(OC(C)=O)c2ccc(F)c(F)c2c1]

NaOAc (161.8 g, 1972.4 mmol, 1 eq.) was added to a solution of Intermediate 75 (533 g, 1972.4 mmol) in acetic anhydride (3600 mL). The resulting solution was stirred at 130° C. for 1 h. After cooling down to room temperature, the reaction mixture was concentrated under reduced pressure. The residue was diluted with water (1000 mL) and extracted with EtOAc (3×3000 mL). The combined organic layers were dried over Na₂SO₄, filtered, and concentrated. The residue was purified by silica gel chromatography (EtOAc/petroleum ether 0/100 to 30/70) to afford Intermediate 76 (190 g, yield: 33%) as a yellow solid.

### Intermediate 77

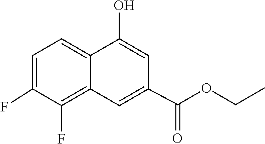 [SMILES: CCOC(=O)c1cc(O)c2ccc(F)c(F)c2c1]

K₂CO₃ (75.86 g, 548.85 mmol, 1.7 eq.) was added to a solution of Intermediate 76 (95 g, 322.85 mmol) in EtOH (1500 mL). The resulting solution was stirred at room temperature for 1 h. The solution was filtered and concentrated under reduced pressure. Aqueous HCl (0.5 M, 500 mL) was added to the residue and the mixture was extracted with EtOAc (3×2000 mL). The combined organic layers were dried over Na₂SO₄, filtered, and concentrated to afford Intermediate 77 (70.4 g, yield: 86%) as a yellow solid, used without further purification.

### Intermediate 78

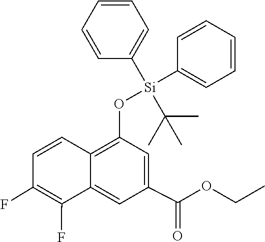 [SMILES: CCOC(=O)c1cc(O[Si](c2ccccc2)(c2ccccc2)C(C)(C)C)c2ccc(F)c(F)c2c1]

Tert-butylchlorodiphenylsilane (92.066 g, 334.955 mmol, 1.2 eq.) and DMAP (6.820 g, 55.826 mmol, 0.2 eq.) were added to a solution of Intermediate 77 (70.4 g, 279.129 mmol) in THE (1500 mL) under nitrogen atmosphere. Imidazole (28.471 g, 418.694 mmol, 1.5 eq.) was then added. The resulting solution was stirred at 50° C. for 16 h. After cooling down to room temperature, the reaction was quenched with water (500 mL). The resulting mixture was extracted with EtOAc (3×1000 mL). The combined organic layers were dried over Na₂SO₄, filtered, and concentrated. The residue was purified by silica gel chromatography (EtOAc/petroleum ether 0/100 to 20/80) to afford Intermediate 78 (114 g, yield: 83%) as a yellow solid.

### Intermediate 79

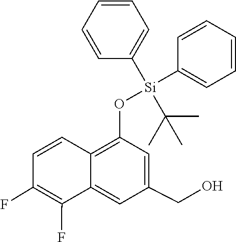 [SMILES: CC(C)(C)[Si](Oc1cc(CO)cc2c(F)c(F)ccc12)(c1ccccc1)c1ccccc1]

LiAlH₄ (10.596 g, 278.835 mmol, 1.2 eq.) dissolved in THE (200 mL) was added to a solution of Intermediate 78 (114 g, 232.362 mmol) in THE (1500 mL) at 0° C. The resulting solution was stirred at room temperature for 1 h. The reaction was quenched by addition of sodium sulfate decahydrate. The resulting mixture was filtered and the filter cake was washed with EtOAc (3×1000 mL). The combined organic layers were concentrated to afford Intermediate 79 (94.6 g, yield: 91%) as a white solid, used without further purification.

### Intermediate 80

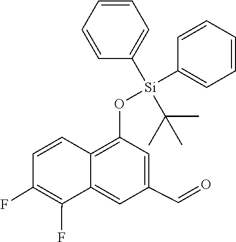 [SMILES: CC(C)(C)[Si](Oc1cc(C=O)cc2c(F)c(F)ccc12)(c1ccccc1)c1ccccc1]

Dess-Martin periodinane (CAS [87413-09-0], 267.773 g, 631.331 mmol, 3 eq.) was added to a solution of Intermediate 79 (94.4 g, 210.444 mmol) in DCM (1500 mL). The resulting mixture was stirred at room temperature for 1 h. The reaction was quenched by addition of saturated aqueous sodium thiosulfate (1000 mL). The resulting mixture was extracted with DCM (3×2000 mL). The combined organic layers were dried over Na₂SO₄, filtered, and concentrated. The residue was purified by silica gel chromatography (petroleum ether/EtOAc 100/0 to 50/50) to afford Intermediate 80 (70 g, yield: 74%) as a white solid.

### Intermediate 81

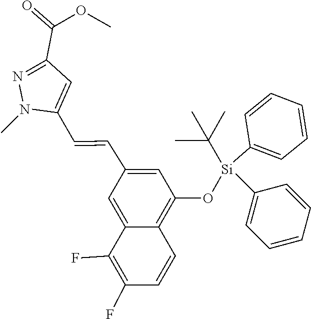 [SMILES: COC(=O)c1cc(/C=C/c2cc(O[Si](c3ccccc3)(c3ccccc3)C(C)(C)C)c3ccc(F)c(F)c3c2)n(C)n1]

Intermediate 73 (61.794 g, 137.047 mmol, 1.2 eq.) was added to a mixture of Intermediate 80 (51 g, 114.206 mmol) in THF (2 L). NaH (60% in mineral oil, 6.8 g, 171.309 mmol, 1.5 eq.) was added to the reaction mixture at 0° C. and the mixture was stirred at room temperature for 40 min. The reaction was quenched by addition of saturated aqueous NH₄Cl (2 L). The mixture was extracted with EtOAc (3×1 L). The combined organic layer was dried over Na₂SO₄, filtered, and concentrated under reduced pressure. The residue was purified by column chromatography on silica gel (petroleum ether/EtOAc 8/1) to afford Intermediate 81 (59 g, yield: 88%) as a white solid.

### Intermediate 82

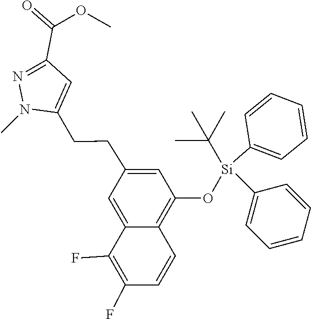 [SMILES: COC(=O)c1cc(CCc2cc(O[Si](c3ccccc3)(c3ccccc3)C(C)(C)C)c3ccc(F)c(F)c3c2)n(C)n1]

Pd/C (10%, 10 g, 0.17 eq.) was added to a solution of Intermediate 81 (58 g, 99.535 mmol) in EtOAc (1 L) and THF (200 mL). The mixture was stirred at 40° C. for 16 h under hydrogen atmosphere. The reaction mixture was filtered through a celite pad and the filtrate was concentrated under reduced pressure. The residue was purified by column chromatography (petroleum ether/EtOAc 5/1) to afford Intermediate 82 (38 g, yield: 65%) as a colorless oil, used without further purification.

### Intermediate 83

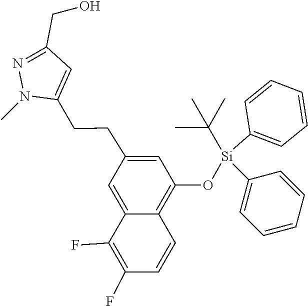 [SMILES: Cn1nc(CO)cc1CCc1cc(O[Si](c2ccccc2)(c2ccccc2)C(C)(C)C)c2ccc(F)c(F)c2c1]

LiAlH₄ (2.89 g, 75.93 mmol, 1.2 eq.) dissolved in THF (20 mL) was added to a solution of Intermediate 82 (37 g, 63.277 mmol) in THE (240 mL) at 0° C. The resulting solution was stirred at room temperature for 1 h. The reaction was quenched by addition of sodium sulfate decahydrate. The resulting mixture was filtered and the filter cake was washed with EtOAc (3×200 mL). The combined organic layer was concentrated and the residue was triturated with petroleum ether and diethyl ether to afford Intermediate 83 as a white solid (15.5 g, yield: 41%), used without further purification.

### Intermediate 84

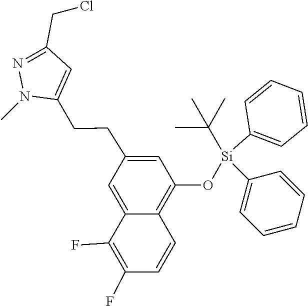 [SMILES: Cn1nc(CCl)cc1CCc1cc(O[Si](c2ccccc2)(c2ccccc2)C(C)(C)C)c2ccc(F)c(F)c2c1]

A solution of Intermediate 83 (1.0 g, 1.70 mmol) in dry DCM (15 mL) was cooled to 0° C. under nitrogen atmosphere. SOCl₂ (0.141 mL, 1.95 mmol, 1.15 eq.) was added dropwise and the reaction mixture was stirred at room temperature for 1 h. The reaction mixture was diluted with DCM (35 mL) and saturated aqueous NaHCO₃ (15 mL). The layers were separated and the organic one was washed with saturated aqueous NaHCO₃ (15 mL) and brine (15 mL). The organic layer was dried over MgSO₄, filtered, and concentrated under reduced pressure to give Intermediate 84 (1030 mg, yield: 98%) as a colorless paste, used without further purification.

### Intermediate 85

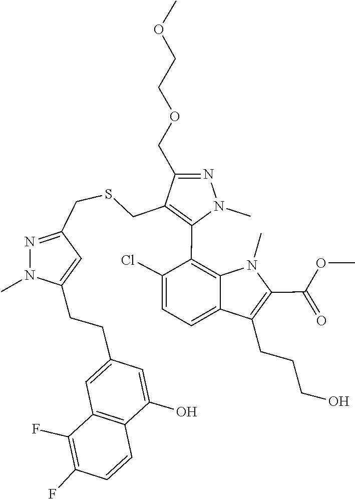

Intermediate 17 (500 mg, 0.77 mmol) and Intermediate 84 (529 mg, 0.92 mmol, 1.2 eq.) were dissolved in MeOH (9 mL) and THF (3 mL). K₂CO₃ (212 mg, 1.53 mmol, 2 eq.) was then added and the reaction mixture was stirred at room temperature for 16 h. The reaction mixture was concentrated under reduced pressure and the residue was partitioned between water (25 mL) and EtOAc (50 mL). The layers were separated and the aqueous layer was extracted with EtOAc (25 mL). The combined organic layer was dried over MgSO₄, filtered, and concentrated under reduced pressure. The residue was dissolved in MeOH (10 mL) and PTSA·H₂O (437 mg, 2.30 mmol, 3 eq.) was added. The reaction mixture was stirred at room temperature for 1 h. The reaction mixture was concentrated under reduced pressure. The residue was dissolved in DCM (100 mL) and washed with saturated aqueous NaHCO₃ (50 mL). The aqueous layer was extracted with DCM (50 mL). The combined organic layer was dried over MgSO₄, filtered, and concentrated under reduced pressure. The residue was purified by flash column chromatography on silica gel (80 g, gradient: DCM/MeOH (NH₃) 100/0 to 96/4) to afford Intermediate 85 (330 mg, yield: 54%) as a white solid.

### Intermediate 86 and Intermediate 87

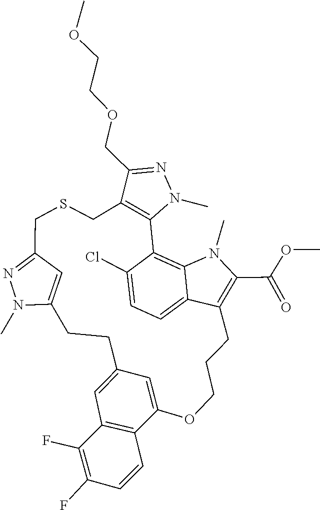 [0435] Intermediate 86: Rₐ or Sₐ; one atropisomer but absolute stereochemistry undetermined
Intermediate 87: Sₐ or Rₐ; one atropisomer but absolute stereochemistry undetermined

A solution of Intermediate 85 (330 mg, 0.414 mmol) and di-tert-butyl azodicarboxylate (191 mg, 0.829 mmol, 2 eq.) in toluene (9 mL) and THE (2 mL) was added with a syringe pump (0.1 mL/min) to a solution of triphenylphosphine (217 mg, 0.829 mmol, 2 eq.) in toluene (9 mL) stirring at 70° C. Once the addition was complete, the reaction was allowed to cool down to room temperature and volatiles were removed under reduced pressure. The residue was purified by flash column chromatography on silica gel (40 g, gradient: EtOAc/MeOH 100/0 to 98/2) followed by preparative SFC (Stationary phase: Chiralpak Daicel IG 20×250 mm, Mobile phase: CO₂, iPrOH+0.4% iPrNH₂) to afford Intermediate 86 (71 mg, yield: 22%) and Intermediate 87 (67 mg, yield: 21%).

### Intermediate 88

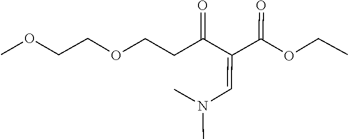 [SMILES: CCOC(=O)/C(=C/N(C)C)C(=O)CCOCCOC]

A mixture of ethyl 5-(2-methoxyethoxy)-3-oxopentanoate (CAS [1350526-27-0], 85 g, 389.467 mmol) in DMF-DMA (58.011 g, 486.834 mmol, 1.25 eq.) was stirred at room temperature for 3 h. The reaction mixture was concentrated under reduced pressure to afford Intermediate 88 (100 g, yield: 94%) as a yellow oil, used without further purification.

### Intermediate 89

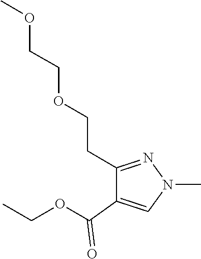

Methylhydrazine sulfate (63.1 g, 437.6 mmol, 1.04 eq.) and sodium acetate (77.3 g, 942.5 mmol, 2.24 eq.) were added to a solution of Intermediate 88 (115 g, 420.7 mmol) in EtOH (1500 mL). The reaction mixture was stirred at 90° C. for 16 h. Water (500 mL) was added and the mixture was extracted with DCM (800 mL×3). The organic layer was washed with brine (1000 mL). The combined organic layer was concentrated under reduced pressure and the residue was purified by flash column chromatography (petroleum ether/EtOAc 3/1) to afford Intermediate 89 (48 g, yield: 44%) as a yellow oil.

### Intermediate 90

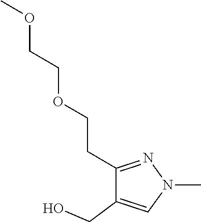

LiAlH₄ (3.558 g, 93.64 mmol, 1.2 eq.) was added to a solution of Intermediate 89 (20 g, 78.03 mmol) in THF (300 mL) at 0° C. The reaction mixture was stirred at room temperature for 1 h. The reaction was quenched by addition of sodium sulfate decahydrate (30 g). The solid was filtered off and the filtrate was concentrated under reduced pressure to afford Intermediate 90 (16 g, yield: 96%) as a yellow oil, used without further purification.

### Intermediate 91

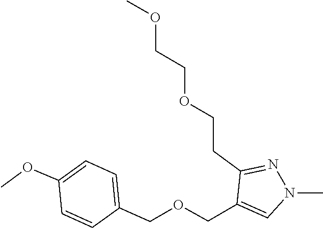

NaH (60% in mineral oil, 3.734 g, 93.34 mmol, 1.25 eq.) was added to a solution of Intermediate 90 (16 g, 74.68 mmol) in DMF (300 mL) and the mixture was stirred at 0° C. for 10 min and at room temperature for 70 min. 4-Methoxybenzylchloride (15.20 g, 97.08 mmol, 1.3 eq.) and KI (1.240 g, 7.47 mmol, 0.1 eq.) were added and the reaction mixture was stirred at room temperature for 16 h. Water (200 mL) was added and the mixture was extracted with EtOAc (200 mL×3). The organic layer was washed with water (100 mL×2) and brine (200 mL), dried over Na₂SO₄, and evaporated. The residue was purified by flash column chromatography (DCM/MeOH 10/1) to afford Intermediate 91 (16 g, yield: 45%) as a yellow oil.

### Intermediate 92

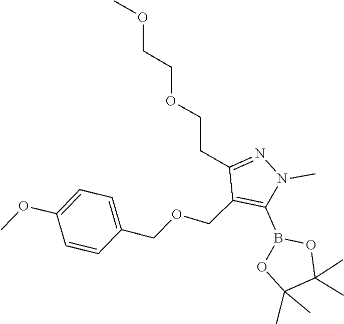

n-Butyllithium (23.9 mL, 59.8 mmol, 2 eq.) was added to a solution of Intermediate 91 (10 g, 29.9 mmol) in THF (100 mL) and the reaction mixture was stirred for 1 h at −78° C. 2-Isopropoxy-4,4,5,5-tetramethyl-1,3,2-dioxaborolane (8.9 g, 47.8 mmol, 1.6 eq.) was added and stirring was continued for 30 min at −78° C. and at room temperature for 1 h. The reaction mixture was added to saturated aqueous NH₄Cl (100 mL). The mixture was extracted with EtOAc (50 mL×3). The organic layer was washed with brine, dried over Na₂SO₄, and evaporated to afford Intermediate 92 (16 g, yield: 45%) as a yellow oil, used without further purification.

### Intermediate 93

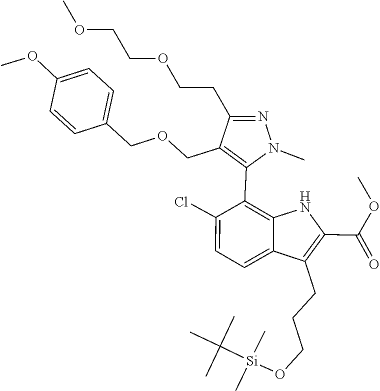 [SMILES: COCCOCCc1nn(C)c(-c2c(Cl)ccc3c(CCCO[Si](C)(C)C(C)(C)C)c(C(=O)OC)[nH]c23)c1COCc1ccc(OC)cc1]

A mixture of Intermediate 92 (3.996 g, 8.680 mmol, 2 eq.), Intermediate 1 (2 g, 4.340 mmol), Palladacycle Gen. 3 (CAS [1447963-73-6], 350 mg, 0.434 mmol, 0.1 eq.) and Na₂CO₃ (1380 mg, 13.018 mmol, 3 eq.) in dioxane (24 mL) and water (3 mL) was stirred at 55° C. for 30 min under nitrogen atmosphere in a microwave oven. The mixture was diluted with water (80 mL) and was extracted with EtOAc (100 mL×3). The organic layer was washed with brine (200 mL), dried over Na₂SO₄, and evaporated. The residue was purified with flash column chromatography (DCM/MeOH 10/1). to afford Intermediate 93 (11.6 g, 59% pure) as a yellow oil.

### Intermediate 94

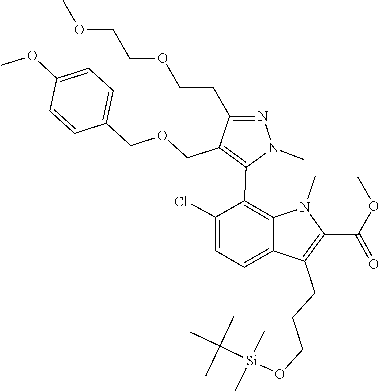 [SMILES: COCCOCCc1nn(C)c(-c2c(Cl)ccc3c(CCCO[Si](C)(C)C(C)(C)C)c(C(=O)OC)n(C)c23)c1COCc1ccc(OC)cc1]

Methyl iodide (4.61 g, 32.476 mmol, 2 eq.) and Cs₂CO₃ (10.58 g, 32.476 mmol, 2 eq.) were added to a solution of Intermediate 93 (11.6 g, 16.238 mmol) in DMF (100 mL). The reaction mixture was stirred at room temperature for 16 h. The reaction mixture was diluted with water (80 mL) and extracted with EtOAc (80 mL×3). The organic layer was washed with brine, dried over Na₂SO₄, and evaporated. The residue was purified by flash column chromatography (petroleum ether/EtOAc 1/2) to afford Intermediate 94 (4.1 g, yield: 32%) as a red oil.

### Intermediate 95

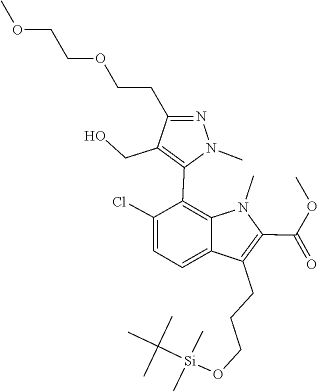 [SMILES: COCCOCCc1nn(C)c(-c2c(Cl)ccc3c(CCCO[Si](C)(C)C(C)(C)C)c(C(=O)OC)n(C)c23)c1CO]

DDQ (2.665 g, 11.738 mmol, 1.5 eq.) and water (13 mL) were added to a solution of Intermediate 94 (5.7 g, 7.826 mmol) in DCM (130 mL). The reaction mixture was stirred at room temperature for 2 h. The reaction mixture was diluted with DCM (50 mL) and washed with saturated aqueous NaHCO₃ (100 mL). The aqueous layer was extracted by DCM (100 mL×3). The combined organic layer was washed with brine (200 mL), dried over Na₂SO₄, filtered, and concentrated under reduced pressure. The residue was purified by flash column chromatography (DCM/MeOH 10/1) to afford Intermediate 95 (4.0 g, yield: 84%) as a brown oil.

### Intermediate 96

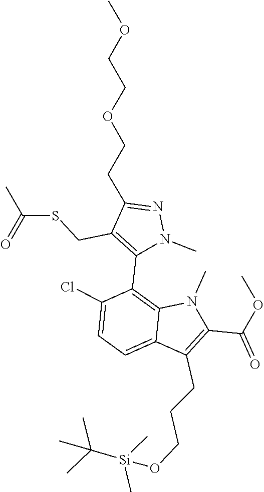 [SMILES: COCCOCCc1nn(C)c(-c2c(Cl)ccc3c(CCCO[Si](C)(C)C(C)(C)C)c(C(=O)OC)n(C)c23)c1CSC(C)=O]

A solution of Intermediate 95 (1 g, 1.644 mmol) in DCM (50 mL) was degassed by bubbling nitrogen through for 10 min. Et₃N (0.5 g, 4.932 mmol, 3 eq.) and MsCl (0.57 g, 4.932 mmol, 3 eq.) were added at 0° C. under nitrogen atmosphere and the reaction mixture was stirred for 10 min at 0° C. A solution of potassium thioacetate (1.88 g, 16.441 mmol, 10 eq.) in DMF (20 mL), degassed by bubbling nitrogen through for 20 min, was added to the reaction mixture and it was stirred at room temperature for 30 min. The mixture was diluted with water (50 mL) and extracted with DCM (50 mL×3). The combined organic layer was washed with brine (100 mL), dried over Na₂SO₄, filtered, and concentrated under reduced pressure. The residue was purified by flash column chromatography (petroleum ether/EtOAc 5/1) to afford Intermediate 96 (1.05 g, yield: 96%) as a yellow oil.

### Intermediate 97

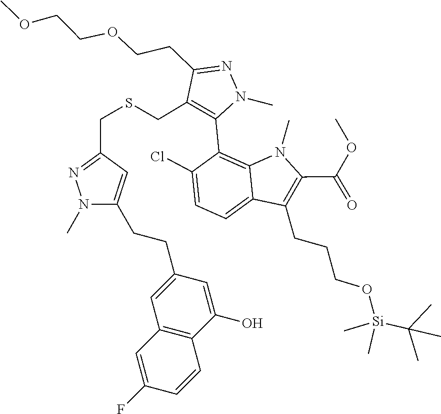 [SMILES: COCCOCCc1nn(C)c(-c2c(Cl)ccc3c(CCCO[Si](C)(C)C(C)(C)C)c(C(=O)OC)n(C)c23)c1CSCc1cc(CCc2cc(O)c3ccc(F)cc3c2)n(C)n1]

A solution of Intermediate 96 (1 g, 1.50 mmol) and Intermediate 49 (0.92 g, 1.65 mmol, 1.1 eq.) in MeOH (100 mL) was stirred at room temperature for 10 min under nitrogen atmosphere. K₂CO₃ (0.415 g, 3.00 mmol, 2 eq.) was added and the reaction mixture was stirred at room temperature for 16 h under nitrogen atmosphere. The mixture was concentrated under reduced pressure. The residue was diluted with water (50 mL) and this mixture was extracted with EtOAc (50 mL×3). The combined organic layer was washed with brine (100 mL), dried over Na₂SO₄, filtered, and concentrated under reduced pressure to afford Intermediate 97 (1.45 g, assumed quantitative) as a yellow oil, used without further purification.

### Intermediate 98

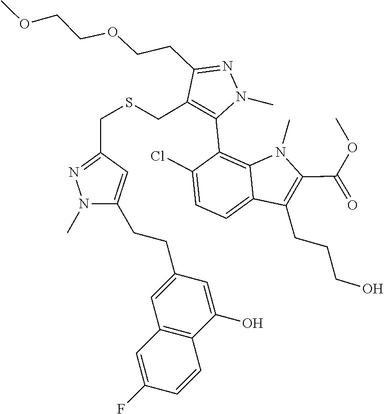 [SMILES: COCCOCCc1nn(C)c(-c2c(Cl)ccc3c(CCCO)c(C(=O)OC)n(C)c23)c1CSCc1cc(CCc2cc(O)c3ccc(F)cc3c2)n(C)n1]

PTSA·H₂O (353 mg, 1.85 mmol, 1.2 eq.). was added to a solution of Intermediate 97 (1.4 g, 1.54 mmol) in MeOH (150 mL). The reaction mixture was stirred at room temperature for 1 h. The mixture was diluted with water (200 mL) and extracted with EtOAc (200 mL×3). The combined organic layer was washed with brine (200 mL), dried over Na₂SO₄, filtered, and concentrated under reduced pressure. The residue was purified by flash column chromatography (DCM/MeOH 10/1) to afford Intermediate 98 (1 g, yield: 82%) as a yellow oil.

### Intermediate 99 and Intermediate 100

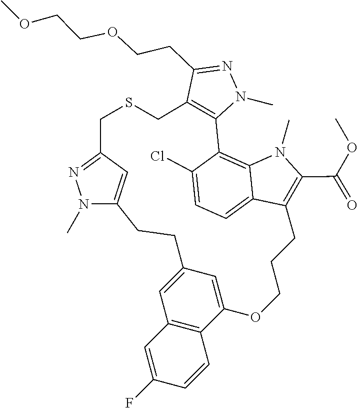 [SMILES: COCCOCCc1nn(C)c2c1CSCc1cc(n(C)n1)CCc1cc(c3ccc(F)cc3c1)OCCCc1c(C(=O)OC)n(C)c3c-2c(Cl)ccc13] [0461] Intermediate 99: Rₐ or Sₐ; one atropisomer but absolute stereochemistry undetermined
Intermediate 100: Sₐ or Rₐ; one atropisomer but absolute stereochemistry undetermined

A solution of Intermediate 98 (900 mg, 1.14 mmol) and di-tert-butyl azodicarboxylate (1.05 g, 4.54 mmol, 4 eq.) in toluene (25 mL) and THF (5 mL) was stirred at room temperature for 10 min under nitrogen atmosphere. This solution was added dropwise over 10 min to a solution of triphenylphosphine (1.19 g, 4.54 mmol, 4 eq.) in toluene (25 mL) at 70° C. under nitrogen atmosphere. After addition, the reaction mixture was further stirred at the same temperature for 30 min. The mixture was then concentrated under reduced pressure and the residue was purified by reverse phase flash chromatography (Column: C18 spherical, 20-35 um, 100 A, 330 g; Mobile Phase A: ACN, Mobile Phase B: H₂O (0.05% 0.5 M NH₄HCO₃—H₂O), Gradient: 30% to 100% in 30 min) followed by preparative chiral HPLC (Column: Chiralpak IF, 2*25 cm, 5 μm; Mobile Phase A: hexane:DCM=3:1 (0.5% 2 M NH₃-MeOH), Mobile Phase B: EtOH; Gradient: 50% B to 50% B in 14 min) to afford Intermediate 99 (60 mg, yield: 7%) and Intermediate 100 (70 mg, yield: 8%).

### Intermediate 101

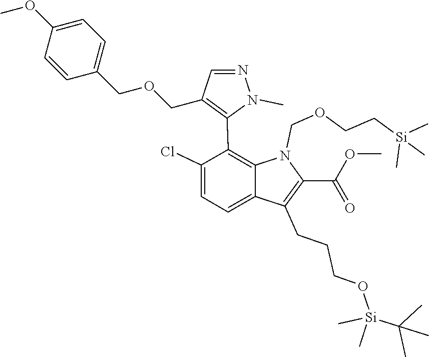 [SMILES: COC(=O)c1c(CCCO[Si](C)(C)C(C)(C)C)c2ccc(Cl)c(-c3c(COCc4ccc(OC)cc4)cnn3C)c2n1COCC[Si](C)(C)C]

Intermediate 24 (7.13 g, 9.67 mmol) was dissolved in dry DMF (200 mL) under nitrogen atmosphere and this solution was cooled to 0° C. NaH (60% dispersion in mineral oil, 425 mg, 10.63 mmol, 1.1 eq.) was added and the solution was stirred for 30 min at 0° C. 2-(trimethylsilyl)ethoxymethyl chloride (2.05 mL, 11.60 mmol, 1.2 eq.) was added and the reaction mixture was stirred at room temperature for 1.5 h. The reaction mixture was poured into brine and the mixture was extracted with EtOAc. The aqueous layer was extracted again twice with EtOAc. The combined organic layer was dried on MgSO₄, filtered, and evaporated. The residue was purified by column chromatography (Biotage Sfar 50 g; eluent: (EtOAc/EtOH 3/1):heptanes 0:100 to 40:60) to give Intermediate 101 (5.35 g, yield: 75%) as a black oil.

### Intermediate 102, Intermediate 102a, and Intermediate 102b

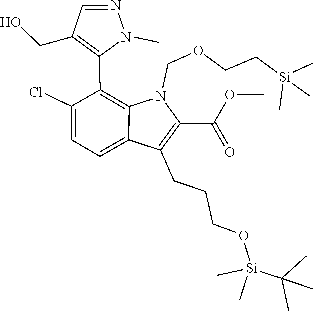 [SMILES: COC(=O)c1c(CCCO[Si](C)(C)C(C)(C)C)c2ccc(Cl)c(-c3c(CO)cnn3C)c2n1COCC[Si](C)(C)C] [0467] Intermediate 102: mixture of atropisomers
Intermediate 102a: Sₐ or Rₐ; one atropisomer but absolute stereochemistry undetermined
Intermediate 102b: Rₐ or Sₐ; one atropisomer but absolute stereochemistry undetermined

2,3-Dichloro-5,6-dicyano-1,4-benzoquinone (232 mg, 1.02 mmol, 1.5 eq.) was added to a solution of Intermediate 101 (703 mg, 0.68 mmol) in DCM (15 mL) and water (2 mL). The reaction mixture was stirred at room temperature for 3 h. The reaction mixture was diluted with DCM and saturated aqueous NaHCO₃ and the layers were separated. The aqueous layer was extracted again with DCM. The combined organic layer was dried by filtration on Extrelut NT3, and evaporated. The residue was purified by column chromatography (Biotage Sfar 25 g; (EtOAc/EtOH 3/1):heptanes 0:100->40:60) to give Intermediate 102 (406 mg, 84% pure, yield: 80%) as a black oil.

In another experiment, a 3.6 g batch of Intermediate 102 was separated into its atropisomers by preparative SFC (Stationary phase: Chiralpak Daicel IC 20×250 mm, Mobile phase: CO₂, EtOH+0.4% iPrNH₂) to give Intermediate 102a (1.24 g) and Intermediate 102b (1.22 g).

### Intermediate 103

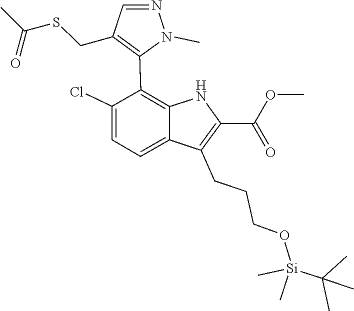 [SMILES: COC(=O)c1[nH]c2c(-c3c(CSC(C)=O)cnn3C)c(Cl)ccc2c1CCCO[Si](C)(C)C(C)(C)C]

Potassium thioacetate (1376 mg, 12.05 mmol, 5 eq.) was dissolved in DMF (previously degassed with nitrogen). Molecular sieves (500 mg) were added to this solution (solution A) and it was kept under nitrogen. DIPEA (0.623 mL, 3.62 mmol, 1.5 eq.) was added to a solution of Intermediate 102 (1.21 g, 2.41 mmol) in dry DCM (15 mL) at 0° C. under nitrogen atmosphere and this solution was degassed with nitrogen for 3 min. MsCl (0.225 mL, 2.89 mmol, 1.2 eq.) was then added at 0° C. and the reaction mixture (solution B) was stirred at 0° C. for 10 min. Solution B was then added in one portion to solution A, stirring at 30° C. Stirring was continued for 15 min at room temperature. The reaction mixture was diluted with EtOAc and was poured into saturated aqueous NaHCO₃. The combined organic layer was dried with MgSO₄, concentrated in vacuo, and the residue was purified by column chromatography (heptane/(EtOAc:EtOH 3:1) 100/0 to 10/90) to give Intermediate 103 (1.062 g, 80% pure, yield: 64%).

### Intermediate 104

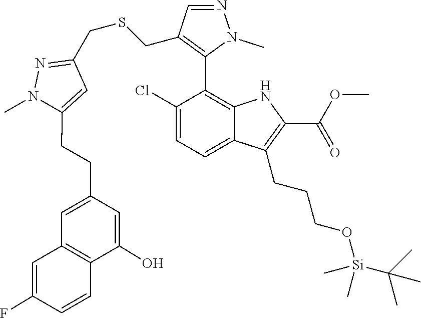 [SMILES: COC(=O)c1[nH]c2c(-c3c(CSCc4cc(CCc5cc(O)c6ccc(F)cc6c5)n(C)n4)cnn3C)c(Cl)ccc2c1CCCO[Si](C)(C)C(C)(C)C]

Intermediate 103 (1.062 g, 1.93 mmol), dissolved in dry and nitrogen-degassed MeOH (17 mL), was added dropwise to a solution of Intermediate 53 (1252 mg, 1.93 mmol, 1 eq.) and K₂CO₃ (320 mg, 2.316 mmol, 1.2 eq.) in nitrogen-degassed MeOH (17 mL) and nitrogen-degassed THE (9 mL). The reaction mixture was stirred at room temperature for 1 h. The reaction mixture was concentrated in vacuo and the residue was diluted with EtOAc and saturated aqueous NaHCO₃. The organic layer was washed with water, dried with MgSO₄, filtered, and evaporated. The residue was purified by column chromatography on silica gel (DCM/MeOH 100/0 to 90/10) to give Intermediate 104 (792 mg, 80% pure, yield: 41%) as an orange oil.

### Intermediate 105

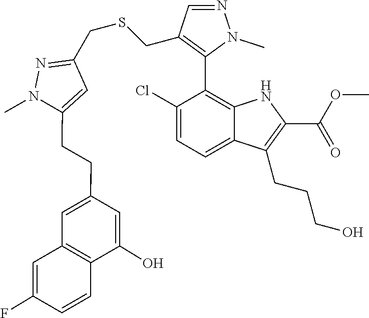 [SMILES: COC(=O)c1[nH]c2c(-c3c(CSCc4cc(CCc5cc(O)c6ccc(F)cc6c5)n(C)n4)cnn3C)c(Cl)ccc2c1CCCO]

PTSA·H₂O (67 mg, 0.391 mmol, 1.2 eq.) was added to a solution of Intermediate 104 (322 mg, 0.326 mmol) in MeOH (2 mL) and the reaction mixture was stirred at room temperature for 30 min. The solvent was evaporated and the residue was taken up in EtOAc and washed with saturated aqueous NaHCO₃. The organic layer was dried with MgSO₄, filtered, and evaporated. The residue was purified by column chromatography on silica gel (DCM/MeOH 9/1) to give Intermediate 105 (321 mg, quantitative yield) as a foam.

### Intermediate 106 and Intermediate 107

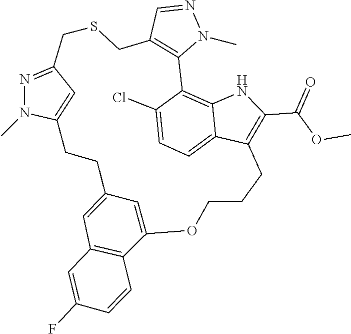

Intermediate 106: Rₐ or Sₐ; one atropisomer but absolute stereochemistry undetermined Intermediate 107: Sₐ or Rₐ; one atropisomer but absolute stereochemistry undetermined A solution of Intermediate 105 (321 mg, 0.475 mmol) and DTBAD (164 mg, 0.712 mmol, 1.5 eq.) in dry nitrogen-degassed toluene (19 mL) and dry nitrogen-degassed THF (1.5 mL) was added dropwise (0.1 mL/min) via a syringe pump to a solution of triphenylphosphine (249 mg, 0.949 mmol, 2 eq.) in nitrogen-degassed dry toluene (19 mL), while stirring at 70° C. under nitrogen atmosphere. After the addition was complete, stirring was continued at 70° C. for 10 min. The solvents were evaporated and the residue was purified by preparative HPLC (Stationary phase: RP XBridge Prep C18 OBD-10 μm, 50×150 mm, Mobile phase: 0.25% NH₄HCO₃ solution in water, CH₃CN), followed by preparative SFC (Stationary phase: Chiralpak Diacel AD 20×250 mm, Mobile phase: CO₂, EtOH+0.4% iPrNH₂) to afford Intermediate 106 (45 mg, yield: 14%) and Intermediate 107 (45 mg, yield: 14%)

### Intermediate 108

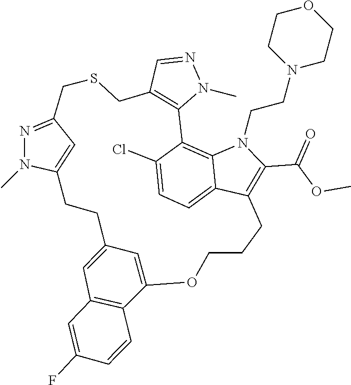 [0481] Rₐ or Sₐ; one atropisomer but absolute stereochemistry undetermined

4-(2-Bromoethyl)morpholine hydrobromide (16.3 mg, 0.059 mmol, 1.5 eq.) was added to a solution of Intermediate 106 (26 mg, 0.039 mmol) and Cs₂CO₃ (32.2 mg, 0.099 mmol, 2.5 eq.) in dry DMF (1 mL) at room temperature. The reaction mixture was stirred at 60° C. overnight. The solvent was evaporated and the residue was taken up in EtOAc and washed with water. The organic layer was evaporated and the residue was purified by column chromatography on silica gel (heptane/(EtOAc:EtOH 3:1) 100/0 to 0/100) to give Intermediate 108 (22 mg, yield: 72%).

### Intermediate 109

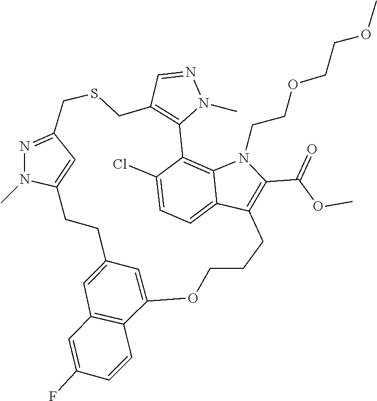 [0484] Rₐ or Sₐ; one atropisomer but absolute stereochemistry undetermined

1-Bromo-2-(2-methoxyethoxy)ethane (7 μL, 0.055 mmol, 1.5 eq.) was added to a solution of Intermediate 106 (24 mg, 0.036 mmol) and Cs₂CO₃ (30 mg, 0.091 mmol, 2.5 eq.) in DMF (1 mL) and the reaction mixture was stirred at 60° C. overnight. The solvent was evaporated and the residue was taken up in EtOAc and washed with water. The organic layer was evaporated and the residue was purified by column chromatography on silica gel (DCM/MeOH 100/0 to 90/10) to give Intermediate 109 (20 mg, yield: 65%).

### Intermediate 110

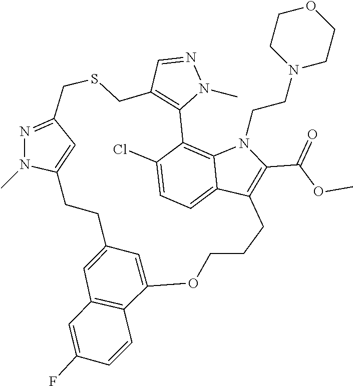 [0487] Sₐ or Rₐ; one atropisomer but absolute stereochemistry undetermined

Intermediate 110 was prepared according to an analogous procedure as for Intermediate 108, starting from Intermediate 107 instead of Intermediate 106.

### Intermediate 111

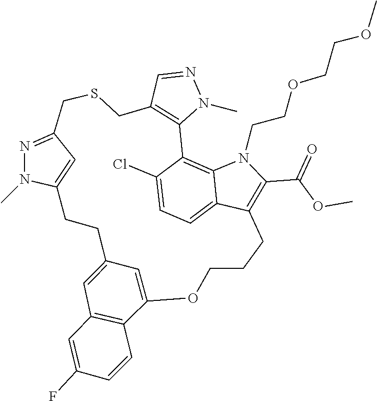 [0490] Sₐ or Rₐ; one atropisomer but absolute stereochemistry undetermined

Intermediate 111 was prepared according to an analogous procedure as for Intermediate 109, starting from Intermediate 107 instead of Intermediate 106.

### Intermediate 112

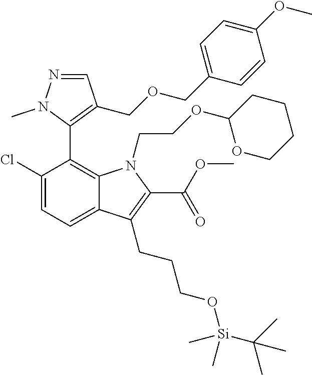

Cs₂CO₃ (7711 mg, 23.67 mmol, 2.5 eq.) and 2-(2-bromoethoxy)tetrahydro-2H-pyran (2.145 mL, 14.2 mmol, 1.5 eq.) were added to a solution of Intermediate 24 (8.28 g, 9.47 mmol) in DMF (73 mL) and the reaction mixture was stirred at 60° C. for 16 h. The reaction mixture was diluted with EtOAc and washed with water. The organic layer was dried with MgSO₄, filtered, and concentrated in vacuo. The residue was purified by column chromatography (Biotage Sfar column 200 g, heptane/(EtOAc:EtOH 3:1)) to give Intermediate 112 (8.4 g, 72% pure, yield: 86%) as a brown oil.

### Intermediate 113

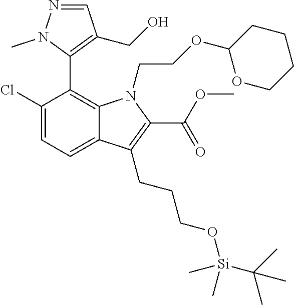 [SMILES: COC(=O)c1c(CCCO[Si](C)(C)C(C)(C)C)c2ccc(Cl)c(-c3c(CO)cnn3C)c2n1CCOC1CCCCO1]

2,3-Dichloro-5,6-dicyano-1,4-benzoquinone (2781 mg, 12.25 mmol, 1.5 eq.) was added to a solution of Intermediate 112 (8.4 g, 8.17 mmol) in DCM (157 mL) and water (18 mL) and the reaction mixture was stirred at room temperature for 2 h. Saturated aqueous NaHCO₃ (20 mL) was added to the reaction mixture and it was stirred vigorously for 5 min. The reaction mixture was diluted with DCM and the layers were separated. The organic layer was washed with brine, dried on MgSO₄, filtered, and evaporated. The residue was purified by column chromatography (Biotage Sfar 200 g; eluent: heptane/(EtOAc:EtOH 3:1) 100/0 to 60/40) to give Intermediate 113 (4.64 g, 82% pure, yield: 75%) as a light brown oil.

### Intermediate 114

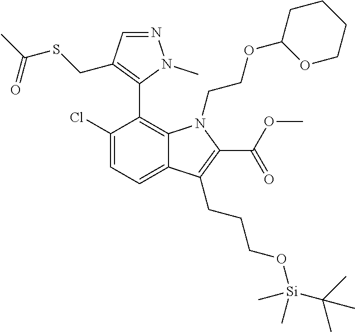 [SMILES: COC(=O)c1c(CCCO[Si](C)(C)C(C)(C)C)c2ccc(Cl)c(-c3c(CSC(C)=O)cnn3C)c2n1CCOC1CCCCO1]

Intermediate 114 was prepared according to an analogous procedure as for Intermediate 103, starting from Intermediate 113 instead of Intermediate 102.

### Intermediate 115

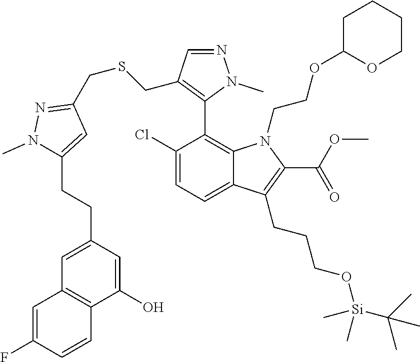 [SMILES: COC(=O)c1c(CCCO[Si](C)(C)C(C)(C)C)c2ccc(Cl)c(-c3c(CSCc4cc(CCc5cc(O)c6ccc(F)cc6c5)n(C)n4)cnn3C)c2n1CCOC1CCCCO1]

Intermediate 114 (3.55 g, 5.23 mmol), dissolved in dry nitrogen-degassed MeOH (42 mL), was added dropwise to a solution of Intermediate 53 (5092 mg, 7.85 mmol, 1.5 eq.) and K₂CO₃ (2170 mg, 15.70 mmol, 3 eq.) in nitrogen-degassed MeOH (47 mL) and nitrogen-degassed THF (23 mL). The reaction mixture was stirred at room temperature for 1 h. The reaction mixture was concentrated in vacuo and the residue was diluted with EtOAc and saturated aqueous NaHCO₃. The organic layer was washed with water, dried with MgSO₄, filtered, and evaporated. The residue was purified by column chromatography on silica gel (heptane/EtOAc 10/1) to give Intermediate 115 (5.62 g, 50% pure, yield: 58%) as an orange oil.

### Intermediate 116

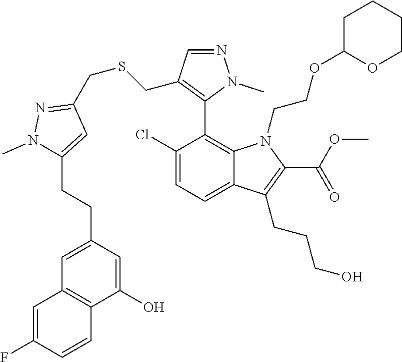 [SMILES: COC(=O)c1c(CCCO)c2ccc(Cl)c(-c3c(CSCc4cc(CCc5cc(O)c6ccc(F)cc6c5)n(C)n4)cnn3C)c2n1CCOC1CCCCO1]

TBAF (1 M in THF, 3.671 mL, 3.671 mmol, 1 eq.) was added to a solution of Intermediate 115 (5.62 g, 3.671 mmol) in dry degassed THF (30 mL). The reaction mixture was stirred at room temperature overnight. The reaction mixture was diluted with water and extracted with EtOAc. The organic layer was dried with MgSO₄, filtered, and evaporated. The residue was purified by column chromatography on silica gel (Biotage Sfar 100 g, heptane/(EtOAc:EtOH 3:1) 100/0 to 20/80) to give Intermediate 116 (2.36 g, 70% pure, yield: 56%) as a dark brown oil.

### Intermediate 117

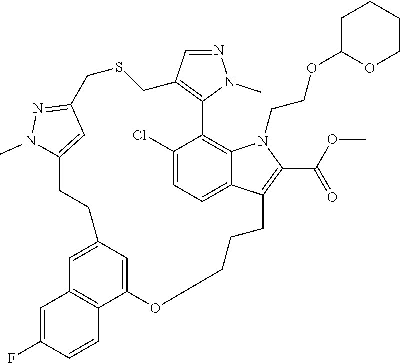 [SMILES: COC(=O)c1c2c3ccc(Cl)c(c3n1CCOC1CCCCO1)-c1c(cnn1C)CSCc1cc(n(C)n1)CCc1cc(c3ccc(F)cc3c1)OCCC2]

A solution of Intermediate 116 (260 mg, 0.226 mmol) and DTBAD (208 mg, 0.905 mmol, 4 eq.) in dry nitrogen-degassed toluene (7 mL) and dry nitrogen-degassed THF (1.3 mL) was added dropwise (0.1 mL/min) via a syringe pump to a solution of triphenylphosphine (237 mg, 0.905 mmol, 4 eq.) in nitrogen-degassed dry toluene (7 mL), while stirring at 70° C. under nitrogen atmosphere. After the addition was complete, stirring was continued at 70° C. for 10 min. The solvents were evaporated and the residue was purified by column chromatography on silica gel (10 g Biotage Sfar column, heptane/(EtOAc:EtOH 3:1) 100/0 to 70/30) to afford Intermediate 117 (120 mg, yield: 61%) as a white solid.

### Intermediate 118

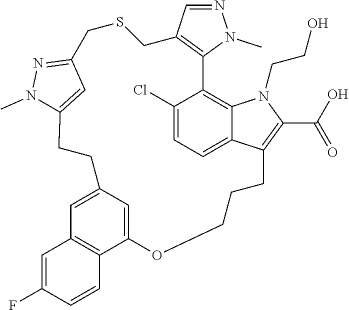 [SMILES: Cn1nc2cc1CCc1cc(c3ccc(F)cc3c1)OCCCc1c(C(=O)O)n(CCO)c3c(c(Cl)ccc13)-c1c(cnn1C)CSC2]

LiOH (33 mg, 1.373 mmol, 10 eq.) was added to a solution of Intermediate 117 (120 mg, 0.137 mmol) in MeOH (5.6 mL), THF (5.6 mL), and water (2.5 mL). The reaction mixture was stirred at 55° C. for 1 h. The solvents were evaporated and the remaining water layer was adjusted to pH 6 using aqueous HCl (1 M). The solution was then extracted with EtOAc. The organic layer was dried with MgSO₄, filtered, and evaporated to give Intermediate 118 (76 mg, yield: 72%), used without any further purification.

### Intermediate 119

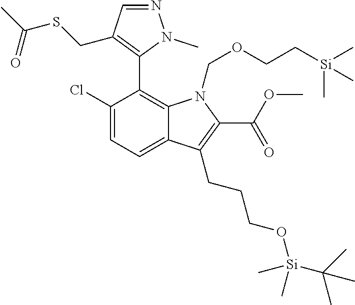 [SMILES: COC(=O)c1c(CCCO[Si](C)(C)C(C)(C)C)c2ccc(Cl)c(-c3c(CSC(C)=O)cnn3C)c2n1COCC[Si](C)(C)C]

DIPEA (0.181 mL, 1.05 mmol, 2 eq.) followed by Ms₂O (110 mg, 0.63 mmol, 1.2 eq.) were added to a solution of Intermediate 102 (389 mg, 0.525 mmol) in DCM (3 mL) at 0° C. under nitrogen atmosphere. The reaction mixture was stirred at 0° C. for 5 min. This solution was then transferred via syringe into a solution of potassium thioacetate (180 mg, 1.575 mmol, 3 eq.) in previously nitrogen-degassed DMF (6 mL). This mixture was stirred for at room temperature for 1 h. The reaction mixture was diluted with EtOAc and washed with water. The organic layer was dried with MgSO₄, filtered, and evaporated. The residue was purified by column chromatography on silica gel (Biotage Sfar 10 g, heptane/(EtOAc:EtOH 3:1)) to give Intermediate 119 (246 mg, yield: 69%).

### Intermediate 120

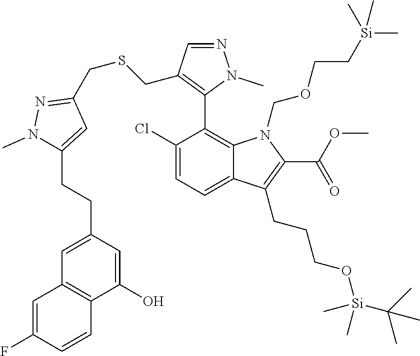 [SMILES: COC(=O)c1c(CCCO[Si](C)(C)C(C)(C)C)c2ccc(Cl)c(-c3c(CSCc4cc(CCc5cc(O)c6ccc(F)cc6c5)n(C)n4)cnn3C)c2n1COCC[Si](C)(C)C]

Intermediate 120 was prepared according to an analogous procedure as for Intermediate 115, starting from Intermediate 119 instead of Intermediate 114.

### Intermediate 121

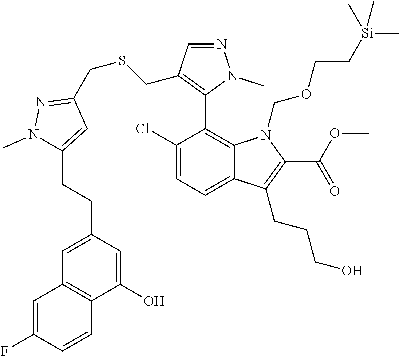

TBAF (1 M in THF, 0.391 mL, 0.391 mmol, 1.2 eq.) was added to a solution of Intermediate 120 (300 mg, 0.326 mmol) in TIE (2.6 mL). The reaction mixture was stirred at room temperature for 2 h. The solvent was evaporated and the residue was purified by column chromatography on silica gel (Biotage Sfar 10 g; heptane/(EtOAc:EtOH 3:1)) to give Intermediate 121 (115 mg, yield: 44%).

### Intermediate 122

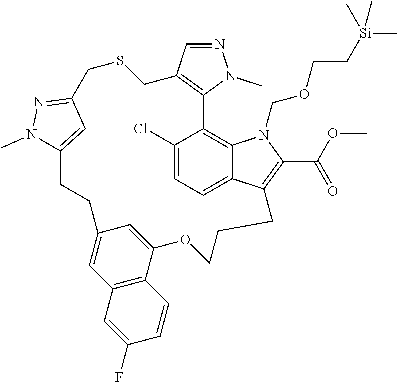

Intermediate 122 was prepared according to an analogous procedure as for Intermediate 117, starting from Intermediate 121 instead of Intermediate 116.

### Intermediate 106 and Intermediate 107 (Alternative Synthesis)

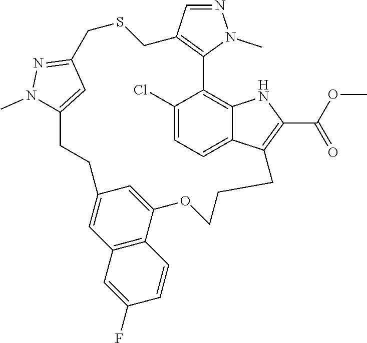 [0515] Intermediate 106: Rₐ or Sₐ; one atropisomer but absolute stereochemistry undetermined
Intermediate 107: Sₐ or Rₐ; one atropisomer but absolute stereochemistry undetermined

TFA (0.116 mL, 1.522 mmol, 20 eq.) was added dropwise to a solution of Intermediate 122 (60 mg, 0.076 mmol) in dry DCM (1 mL) at room temperature. The reaction mixture was stirred at room temperature for 16 h. The solvent was evaporated and the residue was purified by preparative SFC (Stationary phase: Chiralpak Diacel AD 20×250 mm, Mobile phase: CO₂, EtOH+0.4% iPrNH₂) to give impure Intermediate 106 and pure Intermediate 107 (9 mg, yield: 18%). The impure Intermediate 106 was purified again by preparative SFC (Stationary phase: Chiralpak Diacel IC 20×250 mm, Mobile phase: CO₂, EtOH+0.4% iPrNH₂) to give pure Intermediate 106 (10 mg, yield: 20%).

### Intermediate 123

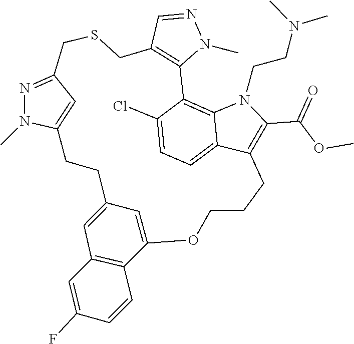 [0519] Intermediate 123: Sₐ or Rₐ; one atropisomer but absolute stereochemistry undetermined

Cs₂CO₃ (99 mg, 0.304 mmol, 2.5 eq.) and 2-bromo-N,N-dimethylethanamine hydrobromide (CAS [2862-39-7], 42 mg, 0.182 mmol, 1.5 eq.) were added to a solution of Intermediate 107 (80 mg, 0.122 mmol) in DMF (4 mL) and the reaction mixture was stirred at 80° C. for 16 h. The reaction mixture was diluted with EtOAc (10 mL) and washed with water (2×5 mL). The combined organic layer was dried with MgSO₄, filtered, and evaporated. The residue was purified by column chromatography (Biotage Sfar 10 g; heptane/(EtOAc:EtOH 3:1) 100/0 to 10/90) to give impure Intermediate 123 (44 mg, 50% pure, yield: 25%) as an oil, used without further purification.

### Preparation of Compounds

### Compound 1

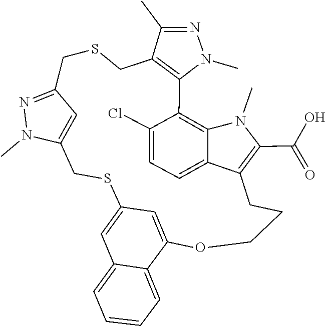 [SMILES: Cc1nn(C)c2c1CSCc1cc(n(C)n1)CSc1cc(c3ccccc3c1)OCCCc1c(C(=O)O)n(C)c3c-2c(Cl)ccc13] [0522] Rₐ or Sₐ; one atropisomer but absolute stereochemistry undetermined

A solution of LiOH·H₂O (9 mg, 3 eq.) in water (0.3 mL) was added to a solution of Intermediate 41 (50 mg, 0.073 mmol) in THF/MeOH (0.9 mL/0.3 mL). The reaction mixture was stirred at 50° C. under nitrogen atmosphere for 3 h. The pH of the reaction mixture was adjusted to 5 with aqueous HCl (1 N) and the mixture was extracted with DCM (10 mL×2). The combined organic layer was washed with brine (5 mL), dried over Na₂SO₄, and evaporated. The residue was purified by preparative HPLC (Column: Xtimate C18 100×30 mm×3 um; eluent: water (0.225% formic acid v/v)/ACN 47/53 to 17/83) to afford Compound 1 (31 mg, yield: 62%) as a white solid after lyophilization.

¹H NMR (400 MHz, MeOH-d₄) δ ppm 2.26 (s, 3H), 2.30-2.52 (m, 2H), 2.82-2.91 (m, 2H), 2.94-3.01 (m, 1H), 3.16-3.30 (m, 2H), 3.44 (s, 3H), 3.50 (s, 3H), 3.59-3.67 (m, 1H), 3.77 (s, 3H), 3.91-4.11 (m, 4H), 4.70 (s, 1H), 6.53 (s, 1H), 7.06 (d, J=8.78 Hz, 1H), 7.30 (s, 1H), 7.45-7.52 (m, 2H), 7.68 (dd, J=6.40, 2.89 Hz, 1H), 7.86 (d, J=8.53 Hz, 1H), 8.13-8.21 (m, 1H)

### Compound 2

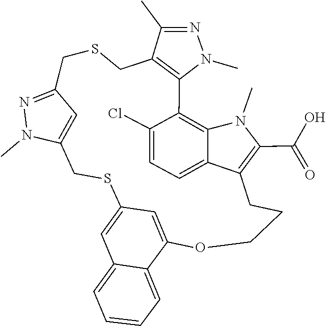 [SMILES: Cc1nn(C)c2c1CSCc1cc(n(C)n1)CSc1cc(c3ccccc3c1)OCCCc1c(C(=O)O)n(C)c3c-2c(Cl)ccc13] [0526] Sₐ or Rₐ; one atropisomer but absolute stereochemistry undetermined

A solution of LiOH (9 mg, 3 eq.) in water (0.3 mL) was added to a solution of Intermediate 42 (50 mg, 0.073 mmol) in THF/MeOH (0.9 mL/0.3 mL). The reaction mixture was stirred at 50° C. under nitrogen atmosphere for 3 h. The pH of the reaction mixture was adjusted to 5 with aqueous HCl (1 N) and the mixture was extracted with DCM (10 mL×2). The combined organic layer was washed with brine (5 mL), dried over Na₂SO₄, and evaporated. The residue was purified by preparative HPLC (Column: Xtimate C18 100×30 mm×3 m; eluent: water (0.225% formic acid v/v)/ACN 47/53 to 17/83) to afford Compound 2 (27 mg, yield 55%) as a white solid after lyophilization.

¹H NMR (400 MHz, METHANOL-d₄) δ ppm 2.26 (s, 3H), 2.31-2.55 (m, 2H), 2.83-2.92 (m, 2H), 2.94-3.02 (m, 1H), 3.18-3.30 (m, 2H), 3.44 (s, 3H), 3.50 (s, 3H), 3.58-3.69 (m, 1H), 3.77 (s, 3H), 3.91-4.12 (m, 4H), 4.70 (s, 1H), 6.53 (d, J=1.25 Hz, 1H), 7.06 (d, J=8.53 Hz, 1H), 7.30 (s, 1H), 7.44-7.52 (m, 2H), 7.64-7.71 (m, 1H), 7.85 (d, J=8.53 Hz, 1H), 8.13-8.20 (m, 1H)

### Compound 3

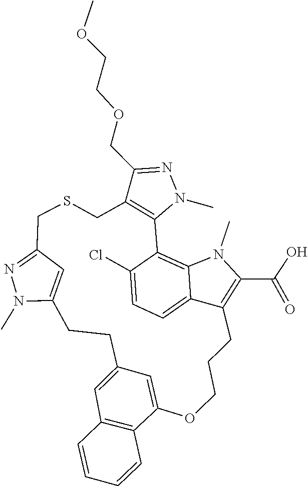 [SMILES: COCCOCc1nn(C)c2c1CSCc1cc(n(C)n1)CCc1cc(c3ccccc3c1)OCCCc1c(C(=O)O)n(C)c3c-2c(Cl)ccc13] [0530] Rₐ or Sₐ; one atropisomer but absolute stereochemistry undetermined

LiOH (75 mg, 15 eq.) was added to a solution of Intermediate 20 (155 mg, 0.21 mmol) in a mixture of MeOH (1.4 mL), THF (1.4 mL) and water (0.7 mL). The reaction mixture was stirred at 60° C. for 3 h. The reaction mixture was cooled down, diluted with MeOH, and purified by preparative HPLC (Stationary phase: RP XBridge Prep C18 OBD—10 μm, 30×150 mm, Mobile phase: 0.25% NH₄HCO₃ solution in water, CH₃CN) to afford Compound 3 (117 mg; yield: 77%) as a white solid.

¹H NMR (400 MHz, DMSO-d₆) δ ppm 2.15-2.36 (m, 2H), 2.76-2.91 (m, 3H), 2.95-3.08 (m, 3H), 3.10-3.22 (m, 2H), 3.24 (s, 3H), 3.36 (d, J=14.1 Hz, 1H), 3.40 (s, 3H), 3.43 (s, 3H), 3.44-3.53 (m, 8H), 3.67-3.76 (m, 1H), 3.77-3.86 (m, 1H), 4.46 (d, J=12.0 Hz, 1H), 4.54 (d, J=12.0 Hz, 1H), 4.92 (s, 1H), 6.18 (s, 1H), 7.05 (d, J=8.6 Hz, 1H), 7.19 (s, 1H), 7.39-7.51 (m, 2H), 7.70-7.76 (m, 1H), 7.81 (d, J=8.6 Hz, 1H), 8.10-8.19 (m, 1H).

### Compound 4

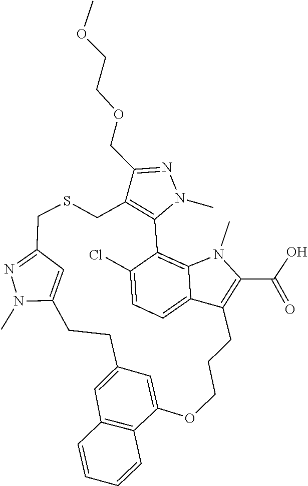 [SMILES: COCCOCc1nn(C)c2c1CSCc1cc(n(C)n1)CCc1cc(c3ccccc3c1)OCCCc1c(C(=O)O)n(C)c3c-2c(Cl)ccc13] [0534] Sₐ or Rₐ; one atropisomer but absolute stereochemistry undetermined

Compound 4 was prepared according to an analogous procedure as for Compound 3, starting from Intermediate 21 instead of Intermediate 20.

¹H NMR (400 MHz, DMSO-d₆) δ ppm 2.14-2.32 (m, 2H), 2.76-2.91 (m, 3H), 2.95-3.09 (m, 3H), 3.10-3.22 (m, 2H), 3.24 (s, 3H), 3.35 (d, J=13.9 Hz, 1H), 3.40 (s, 3H), 3.43 (s, 3H), 3.44-3.53 (m, 8H), 3.68-3.77 (m, 1H), 3.78-3.86 (m, 1H), 4.46 (d, J=11.9 Hz, 1H), 4.54 (d, J=11.9 Hz, 1H), 4.92 (s, 1H), 6.18 (s, 1H), 7.05 (d, J=8.6 Hz, 1H), 7.19 (s, 1H), 7.40-7.52 (m, 2H), 7.70-7.77 (m, 1H), 7.81 (d, J=8.6 Hz, 1H), 8.10-8.18 (m, 1H).

### Compound 5

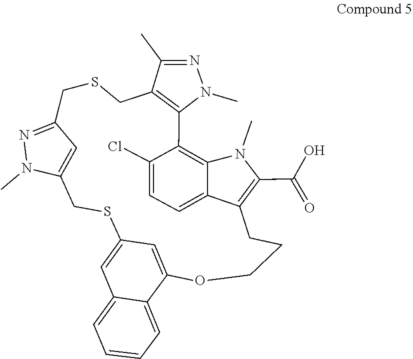 [SMILES: Cc1nn(C)c2c1CSCc1cc(n(C)n1)CSc1cc(c3ccccc3c1)OCCCc1c(C(=O)O)n(C)c3c-2c(Cl)ccc13] [0538] Rₐ or Sₐ; one atropisomer but absolute stereochemistry undetermined

LiOH·H₂O (61 mg, 15 eq.) was added to a stirred solution of Intermediate 30 (65 mg, 0.097 mmol) in THF/MeOH/water 2/1/1 (4 mL) and the reaction mixture was stirred at room temperature overnight. The solvents were evaporated and water (3 mL) was added to the residue. Aqueous HCl (1 M) was added to reach pH˜5. The mixture was extracted with EtOAc (10 mL×3). The combined organic layer was washed by brine, dried with Na₂SO₄, filtered, and evaporated to give Compound 5 (60 mg, yield: 93%) as a white solid.

¹H NMR (400 MHz, METHANOL-d₄) δ ppm 2.41 (br d, J=3.53 Hz, 2H) 2.67-2.80 (m, 2H) 2.87 (d, J=14.55 Hz, 1H) 3.16 (d, J=14.55 Hz, 1H) 3.22 (br dd, J=9.81, 4.96 Hz, 1H) 3.40 (s, 3H) 3.54 (s, 3H) 3.62 (dt, J=13.73, 4.60 Hz, 1H) 3.77 (s, 3H) 3.91-4.07 (m, 4H) 4.67 (s, 1H) 6.52 (s, 1H) 7.06 (d, J=8.60 Hz, 1H) 7.18 (s, 1H) 7.40-7.46 (m, 2H) 7.51 (s, 1H) 7.62 (dd, J=6.50, 2.98 Hz, 1H) 7.85 (d, J=8.60 Hz, 1H) 8.07 (dd, J=6.28, 3.42 Hz, 1H)

### Compound 6

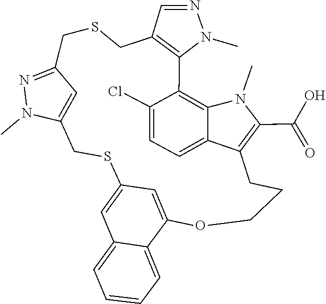 [SMILES: Cn1nc2cc1CSc1cc(c3ccccc3c1)OCCCc1c(C(=O)O)n(C)c3c(c(Cl)ccc13)-c1c(cnn1C)CSC2] [0542] Sₐ or Rₐ; one atropisomer but absolute stereochemistry undetermined

Compound 6 was prepared according to an analogous procedure as for Compound 5, starting from Intermediate 31 instead of Intermediate 30.

¹H NMR (400 MHz, METHANOL-d₄) δ ppm 2.41 (br s, 2H) 2.75 (q, J=14.31 Hz, 2H) 2.87 (d, J=14.31 Hz, 1H) 3.12-3.25 (m, 21H) 3.40 (s, 31H) 3.54 (s, 31H) 3.59-3.68 (m, 1H) 3.77 (s, 3H) 3.92-4.07 (m, 41H) 4.67 (s, 1H) 6.52 (s, 1H) 7.06 (d, J=8.53 Hz, 1H) 7.18 (s, 1H) 7.39-7.47 (m, 2H) 7.51 (s, 1H) 7.62 (dd, J=6.53, 3.01 Hz, 1H) 7.85 (d, J=8.78 Hz, 1H) 8.04-8.11 (m, 1H)

### Compound 7

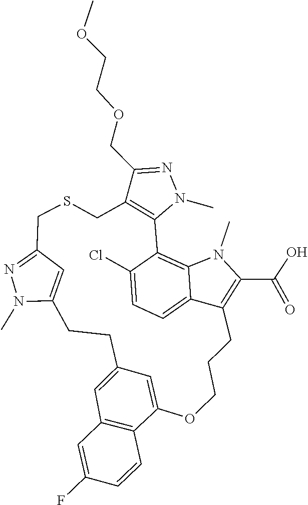 [SMILES: COCCOCc1nn(C)c2c1CSCc1cc(n(C)n1)CCc1cc(c3ccc(F)cc3c1)OCCCc1c(C(=O)O)n(C)c3c-2c(Cl)ccc13] [0546] Sₐ or Rₐ; one atropisomer but absolute stereochemistry undetermined

A solution of LiOH (10 mg, 15 eq.) in water (0.2 mL) was added to a solution of Intermediate 51 (21 mg, 0.028 mmol) in THF (0.3 mL) and MeOH (0.3 mL). The reaction mixture was stirred at 60° C. for 3 h. The reaction mixture was cooled to room temperature and concentrated under reduced pressure. The residue was partitioned between DCM (15 mL) and water (5 mL) and was treated with HCl (1 N in water) until pH 2-3. The organic layer was separated and the aqueous one was extracted with DCM (2×10 mL). The combined organic layer was dried over MgSO₄, filtered, and evaporated. The residue was dissolved in MeOH and evaporated twice to obtain Compound 7 (12 mg, yield: 58%) as an off-white solid.

¹H NMR (400 MHz, DMSO-d₆) δ ppm 2.13-2.34 (m, 2H), 2.76-2.88 (m, 3H), 2.92-3.06 (m, 3H), 3.11-3.21 (m, 2H), 3.23 (s, 3H), 3.33 (d, J=14.5 Hz, 1H), 3.39 (s, 3H), 3.41 (s, 3H), 3.43-3.52 (m, 8H), 3.66-3.75 (m, 1H), 3.76-3.84 (m, 1H), 4.45 (d, J=11.9 Hz, 1H), 4.53 (d, J=11.9 Hz, 1H), 4.91 (s, 1H), 6.12 (s, 1H), 7.08 (d, J=8.6 Hz, 1H), 7.16 (s, 1H), 7.30 (td, J=8.9, 2.6 Hz, 1H), 7.49 (dd, J=10.5, 2.6 Hz, 1H), 7.82 (d, J=8.6 Hz, 1H), 8.18 (dd, J=9.2, 5.9 Hz, 1H).

### Compound 8

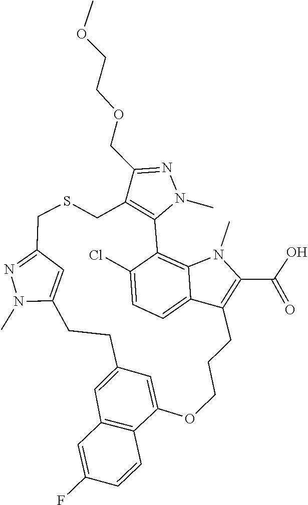 [SMILES: COCCOCc1nn(C)c2c1CSCc1cc(n(C)n1)CCc1cc(c3ccc(F)cc3c1)OCCCc1c(C(=O)O)n(C)c3c-2c(Cl)ccc13] [0550] Rₐ or Sₐ; one atropisomer but absolute stereochemistry undetermined

Compound 8 was prepared according to an analogous procedure as for Compound 7, starting from Intermediate 52 instead of Intermediate 51.

¹H NMR (400 MHz, DMSO-d₆) δ ppm 2.13-2.35 (m, 2H), 2.76-2.88 (m, 3H), 2.92-3.07 (m, 3H), 3.12-3.21 (m, 2H), 3.23 (s, 3H), 3.33 (d, J=14.3 Hz, 1H), 3.39 (s, 3H), 3.41 (s, 3H), 3.43-3.53 (m, 8H), 3.66-3.74 (m, 1H), 3.75-3.84 (m, 1H), 4.45 (d, J=11.9 Hz, 1H), 4.53 (d, J=11.9 Hz, 1H), 4.91 (s, 1H), 6.12 (s, 1H), 7.08 (d, J=8.6 Hz, 1H), 7.16 (s, 1H), 7.30 (td, J=8.9, 2.7 Hz, 1H), 7.49 (dd, J=10.5, 2.7 Hz, 1H), 7.82 (d, J=8.6 Hz, 1H), 8.18 (dd, J=9.2, 5.9 Hz, 1H).

### Compound 9

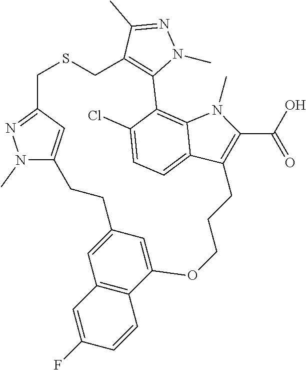 [SMILES: Cc1nn(C)c2c1CSCc1cc(n(C)n1)CCc1cc(c3ccc(F)cc3c1)OCCCc1c(C(=O)O)n(C)c3c-2c(Cl)ccc13] [0554] Rₐ or Sₐ; one atropisomer but absolute stereochemistry undetermined

NaOH (30 mg, 5 eq.) was slowly added to a solution of Intermediate 56 (105 mg, 0.153 mmol) in MeOH/water (1/1, 5 mL). The reaction mixture was stirred at room temperature for 3 h. The reaction mixture was extracted with EtOAc (5 mL×2), and the combined organic layer was washed with brine (5 mL), dried on Na₂SO₄, and evaporated. The residue was purified by preparative HPLC (Column: Boston Green ODS 150×30 mm×5 um; water (0.05% ammonia hydroxide v/v)/ACN 40/60 to 10/90) to afford Compound 9 (46 mg, yield: 45%) as a white solid.

¹H NMR (400 MHz, Chloroform-d) δ ppm 8.28 (dd, J=5.8, 9.2 Hz, 1H), 7.63 (d, J=8.4 Hz, 1H), 7.30 (dd, J=2.1, 10.0 Hz, 1H), 7.22-7.17 (m, 1H), 7.12 (s, 1H), 7.06 (br d, J=8.6 Hz, 1H), 5.54 (br s, 1H), 5.39 (br s, 1H), 3.58 (s, 3H), 3.57-3.51 (m, 1H), 3.51-3.41 (m, 6H), 3.25-3.15 (m, 2H), 3.11 (br s, 3H), 2.98 (br d, J=12.8 Hz, 1H), 2.92-2.77 (m, 5H), 2.29 (s, 5H)

### Compound 10 (Free Base) and Compound 10a (Na Salt)

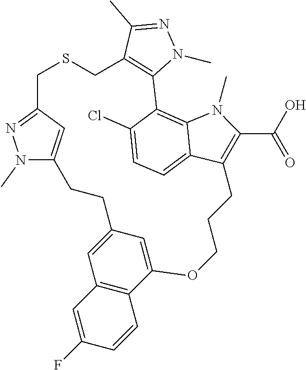 [SMILES: Cc1nn(C)c2c1CSCc1cc(n(C)n1)CCc1cc(c3ccc(F)cc3c1)OCCCc1c(C(=O)O)n(C)c3c-2c(Cl)ccc13] [0558] Sₐ or Rₐ; one atropisomer but absolute stereochemistry undetermined

Compound 10 was prepared according to an analogous procedure as for Compound 9, starting from Intermediate 57 instead of Intermediate 56.

A sample of Compound 10 (29.6 mg, 0.0425 mmol) was dissolved in MeOH (1 mL) and NaOH (1 M in water, 43.5 μL, 0.0435 mmol, 1 eq.) was added. The mixture was stirred for a few min, then volatiles were removed under reduced pressure. The residue was suspended in DIPE (2 mL) and evaporated to dryness. The residue was then triturated with DIPE, filtered, and dried to afford the sodium salt Compound 10a (29 mg, Na salt, yield: 96%) as an off-white solid.

Compound 10: ¹H NMR (400 MHz, Chloroform-d) δ ppm 8.30 (dd, J=5.8, 9.2 Hz, 1H), 7.61 (d, J=8.6 Hz, 1H), 7.30 (dd, J=2.3, 10.0 Hz, 1H), 7.23-7.17 (m, 1H), 7.11 (s, 1H), 7.00 (br d, J=8.6 Hz, 1H), 5.62 (br s, 1H), 5.28 (br s, 1H), 3.60-3.51 (m, 5H), 3.50-3.40 (m, 5H), 3.27-3.12 (m, 5H), 3.04 (br d, J=12.6 Hz, 1H), 2.90-2.75 (m, 5H), 2.29 (s, 5H)

Compound 10a: ¹H NMR (400 MHz, DMSO-d₆) δ ppm 2.16 (s, 3H), 2.19-2.27 (m, 2H), 2.81-2.96 (m, 4H), 2.96-3.08 (m, 2H), 3.12 (d, J=13.8 Hz, 1H), 3.19 (d, J=13.6 Hz, 1H), 3.29 (d, J=13.6 Hz, 1H), 3.35 (s, 3H), 3.39 (s, 3H), 3.40-3.46 (m, 1H), 3.48 (s, 3H), 3.76-3.92 (m, 2H), 4.94 (s, 1H), 6.33 (s, 1H), 6.93 (d, J=8.5 Hz, 1H), 7.18 (s, 1H), 7.29 (td, J=8.9, 2.6 Hz, 1H), 7.48 (dd, J=10.5, 2.6 Hz, 1H), 7.59 (d, J=8.5 Hz, 1H), 8.17 (dd, J=9.2, 5.9 Hz, 1H).

### Compound 11 (Free Base) and Compound 11a (Na Salt)

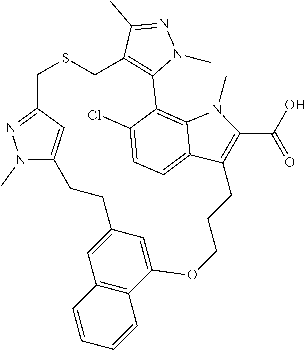 [SMILES: Cc1nn(C)c2c1CSCc1cc(n(C)n1)CCc1cc(c3ccccc3c1)OCCCc1c(C(=O)O)n(C)c3c-2c(Cl)ccc13] [0564] Rₐ or Sₐ; one atropisomer but absolute stereochemistry undetermined

Compound 11 was prepared according to an analogous procedure as for Compound 1, starting from Intermediate 61 instead of Intermediate 41.

A sample of Compound 11 (48 mg, 0.0734 mmol) was suspended in MeOH (3 mL) and NaOH (2.95 mg, 0.074 mmol, 1 eq.) was added. After the mixture became a clear solution, volatiles were removed in vacuo. The residue was triturated with DIPE, filtered, and dried to afford the sodium salt Compound 11a (44 mg, Na salt, yield: 89%) as an off-white solid.

Compound 11: ¹H NMR (400 MHz, Chloroform-d) δ ppm 8.33-8.28 (m, 1H), 7.73-7.68 (m, 1H), 7.63 (d, J=8.4 Hz, 1H), 7.49-7.41 (m, 2H), 7.18 (s, 1H), 6.99 (d, J=8.6 Hz, 1H), 5.64 (s, 1H), 5.30 (s, 1H), 3.57 (s, 3H), 3.54 (br s, 2H), 3.47 (s, 3H), 3.42 (br s, 1H), 3.22 (br s, 2H), 3.17 (br s, 3H), 3.02 (br d, J=13.2 Hz, 1H), 2.86 (br d, J=7.1 Hz, 4H), 2.78 (br d, J=12.8 Hz, 1H), 2.34-2.29 (m, 2H), 2.28 (s, 3H)

Compound 11a: ¹H NMR (400 MHz, DMSO-d₆) δ ppm 2.16 (s, 3H), 2.19-2.29 (m, 2H), 2.81-2.92 (m, 4H), 2.94-3.09 (m, 2H), 3.13 (d, J=13.9 Hz, 1H), 3.20 (d, J=13.4 Hz, 1H), 3.28 (d, J=13.4 Hz, 1H), 3.35 (s, 3H), 3.39 (s, 3H), 3.40-3.46 (m, 1H), 3.49 (s, 3H), 3.78-3.93 (m, 2H), 4.93 (s, 1H), 6.38 (s, 1H), 6.89 (d, J=8.4 Hz, 1H), 7.19 (s, 1H), 7.38-7.48 (m, 2H), 7.57 (d, J=8.4 Hz, 1H), 7.68-7.74 (m, 1H), 8.09-8.15 (m. 1H).

### Compound 12 (Free Base) and Compound 12a (Na Salt)

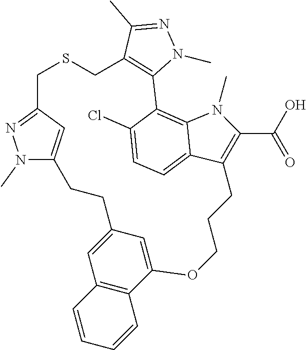 [SMILES: Cc1nn(C)c2c1CSCc1cc(n(C)n1)CCc1cc(c3ccccc3c1)OCCCc1c(C(=O)O)n(C)c3c-2c(Cl)ccc13] [0570] Sₐ or Rₐ; one atropisomer but absolute stereochemistry undetermined

Compound 12 was prepared according to an analogous procedure as for Compound 2, starting from Intermediate 62 instead of Intermediate 42.

A sample of Compound 12 (13.5 mg, 0.021 mmol) was dissolved in MeOH (1 mL) and NaOH (1 M in water, 20.6 μL, 0.0206 mmol, 1 eq.) was added. The mixture was stirred for a few min, then volatiles were removed under reduced pressure. The residue was suspended in DIPE (2 mL) and evaporated to dryness. The residue was triturated with DIPE, filtered, and dried to afford the sodium salt Compound 12a (10 mg, Na salt, yield: 72%) as an off-white solid.

Compound 12: ¹H NMR (400 MHz, Chloroform-d) δ ppm 8.33-8.27 (m, 1H), 7.72-7.67 (m, 1H), 7.63 (d, J=8.6 Hz, 1H), 7.45 (td, J=2.2, 4.9 Hz, 2H), 7.18 (s, 1H), 7.00 (d, J=8.4 Hz, 1H), 5.63 (s, 1H), 5.31 (s, 1H), 3.57 (s, 3H), 3.53 (br d, J=6.0 Hz, 2H), 3.47 (s, 3H), 3.42 (br s, 2H), 3.28-3.19 (m, 2H), 3.16 (s, 3H), 3.02 (br d, J=12.1 Hz, 1H), 2.86 (br d, J=6.8 Hz, 4H), 2.79 (br d, J=12.6 Hz, 1H), 2.33-2.29 (m, 2H), 2.29 (s, 3H)

Compound 12a: ¹H NMR (400 MHz, DMSO-d₆) δ ppm 2.16 (s, 3H), 2.20-2.29 (m, 2H), 2.79-2.95 (m, 4H), 2.97-3.09 (m, 2H), 3.13 (d, J=13.9 Hz, 1H), 3.20 (d, J=13.6 Hz, 1H), 3.27 (d, J=13.9 Hz, 1H), 3.37 (s, 3H), 3.39 (s, 3H), 3.42-3.50 (m, 4H), 3.76-3.92 (m, 2H), 4.90 (s, 1H), 6.35 (br s, 1H), 6.91 (d, J=8.4 Hz, 1H), 7.19 (s, 1H), 7.35-7.51 (m, 2H), 7.61 (d, J=8.4 Hz, 1H), 7.71 (d, J=7.5 Hz, 1H), 8.12 (br d, J=7.9 Hz, 1H).

### Compound 13

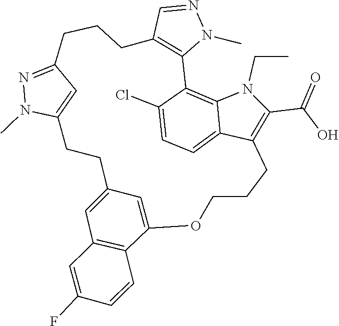 [SMILES: CCn1c(C(=O)O)c2c3ccc(Cl)c(c31)-c1c(cnn1C)CCCc1cc(n(C)n1)CCc1cc(c3ccc(F)cc3c1)OCCC2] [0576] Sₐ or Rₐ; one atropisomer but absolute stereochemistry undetermined

Compound 13 was prepared according to an analogous procedure as for Compound 5, starting from Intermediate 71 instead of Intermediate 30.

¹H NMR (400 MHz, Chloroform-d, 27° C.) δ ppm 0.99 (t, J=7.0 Hz, 3H) 1.39-1.54 (m, 2H) 2.02-2.11 (m, 1H) 2.16-2.45 (m, 5H) 2.78-2.90 (m, 2H) 2.91-3.01 (m, 2H) 3.17-3.26 (m, 1H) 3.27 (s, 3H) 3.61-3.70 (m, 4H) 3.72 (s, 3H) 4.28 (dq, J=14.4, 7.1 Hz, 1H) 5.44 (s, 1H) 5.85 (s, 1H) 7.12 (d, J=8.6 Hz, 2H) 7.20 (td, J=8.8, 2.6 Hz, 1H) 7.31 (dd, J=10.0, 2.5 Hz, 1H) 7.47 (s, 1H) 7.68 (d, J=8.6 Hz, 1H) 8.29 (dd, J=9.1, 5.8 Hz, 1H).

### Compound 14

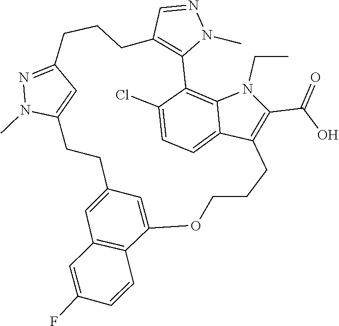 [SMILES: CCn1c(C(=O)O)c2c3ccc(Cl)c(c31)-c1c(cnn1C)CCCc1cc(n(C)n1)CCc1cc(c3ccc(F)cc3c1)OCCC2] [0580] Rₐ or Sₐ; one atropisomer but absolute stereochemistry undetermined

Compound 14 was prepared according to an analogous procedure as for Compound 5, starting from Intermediate 72 instead of Intermediate 30.

¹H NMR (400 MHz, Chloroform-d, 27° C.) δ ppm 0.98 (t, J=7.2 Hz, 3H) 1.42-1.52 (m, 2H) 2.07-2.16 (m, 1H) 2.20-2.41 (m, 5H) 2.80-2.90 (m, 2H) 2.90-3.00 (m, 2H) 3.17-3.28 (m, 1H) 3.32 (s, 3H) 3.62-3.77 (m, 4H) 3.71 (s, 3H) 4.28 (dq, J=14.4, 7.1 Hz, 1H) 5.42 (s, 1H) 5.90 (s, 1H) 7.10 (d, J=8.6 Hz, 1H) 7.12 (s, 1H) 7.20 (td, J=8.8, 2.6 Hz, 1H) 7.30 (dd, J=10.0, 2.5 Hz, 1H) 7.47 (s, 1H) 7.68 (d, J=8.6 Hz, 1H) 8.29 (dd, J=9.1, 5.8 Hz, 1H).

### Compound 15

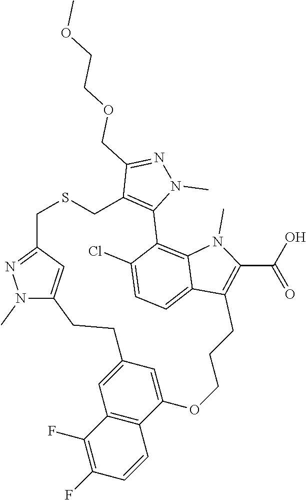 [SMILES: COCCOCc1nn(C)c2c1CSCc1cc(n(C)n1)CCc1cc(c3ccc(F)c(F)c3c1)OCCCc1c(C(=O)O)n(C)c3c-2c(Cl)ccc13] [0584] Rₐ or Sₐ; one atropisomer but absolute stereochemistry undetermined

A solution of LiOH (32.8 mg, 1.368 mmol, 15 eq.) in water (0.7 mL) was added to a solution of Intermediate 86 (71 mg, 0.0912 mmol) in THE (1.0 mL) and MeOH (1.0 mL). The reaction mixture was stirred at 60° C. for 2 h. The reaction mixture was cooled to room temperature, diluted with MeOH, and directly injected into preparative HPLC (Stationary phase: RP XBridge Prep C18 OBD-10 μm, 30×150 mm, Mobile phase: 0.25% NH₄HCO₃ solution in water, CH₃CN). The obtained product was triturated with DIPE and evaporated to give Compound 15 (57 mg, yield: 82%) as a white solid.

¹H NMR (400 MHz, DMSO-d₆) δ ppm 2.14-2.33 (m, 2H), 2.80 (d, J=13.5 Hz, 1H), 2.81-2.93 (m, 2H), 2.97-3.08 (m, 3H), 3.09-3.18 (m, 2H), 3.23 (s, 3H), 3.31 (br d, J=13.3 Hz, 1H), 3.38 (s, 3H), 3.42 (s, 3H), 3.43-3.47 (m, 3H), 3.47-3.53 (m, 5H), 3.67-3.76 (m, 1H), 3.79-3.87 (m, 1H), 4.45 (d, J=11.9 Hz, 1H), 4.54 (d, J=11.9 Hz, 1H), 4.92 (s, 1H), 6.22 (s, 1H), 7.08 (d, J=8.6 Hz, 1H), 7.30 (s, 1H), 7.43-7.52 (m, 1H), 7.79 (d, J=8.6 Hz, 1H), 7.97 (dd, J=9.2, 4.8 Hz, 1H).

### Compound 16

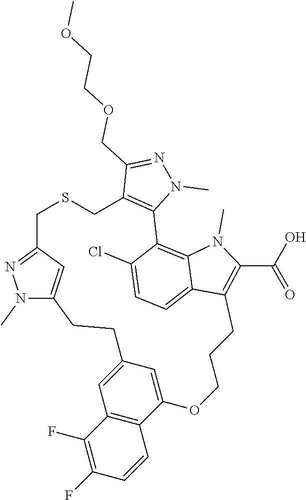 [SMILES: COCCOCc1nn(C)c2c1CSCc1cc(n(C)n1)CCc1cc(c3ccc(F)c(F)c3c1)OCCCc1c(C(=O)O)n(C)c3c-2c(Cl)ccc13] [0588] Sₐ or Rₐ; one atropisomer but absolute stereochemistry undetermined

Compound 16 was prepared according to an analogous procedure as for Compound 15, starting from Intermediate 87 instead of Intermediate 86.

¹H NMR (400 MHz, DMSO-d₆) δ ppm 2.14-2.33 (m, 2H), 2.79 (d, J=13.6 Hz, 1H), 2.80-2.92 (m, 2H), 2.98-3.08 (m, 3H), 3.10-3.18 (m, 2H), 3.23 (s, 3H), 3.30 (br d, J=13.3 Hz, 1H), 3.38 (s, 3H), 3.42 (s, 3H), 3.43-3.47 (m, 3H), 3.48-3.52 (m, 5H), 3.66-3.77 (m, 1H), 3.78-3.87 (m, 1H), 4.45 (d, J=11.9 Hz, 1H), 4.54 (d, J=11.9 Hz, 1H), 4.91 (s, 1H), 6.21 (s, 1H), 7.09 (d, J=8.6 Hz, 1H), 7.30 (s, 1H), 7.43-7.52 (m, 1H), 7.81 (d, J=8.6 Hz, 1H), 7.97 (dd, J=9.2, 4.8 Hz, 1H).

### Compound 17

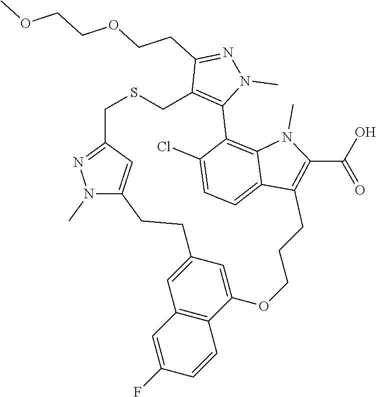 [SMILES: COCCOCCc1nn(C)c2c1CSCc1cc(n(C)n1)CCc1cc(c3ccc(F)cc3c1)OCCCc1c(C(=O)O)n(C)c3c-2c(Cl)ccc13] [0592] Rₐ or Sₐ; one atropisomer but absolute stereochemistry undetermined

LiOH (12 mg, 0.465 mmol, 6 eq.) in water (3 mL) was added to a solution of Intermediate 99 (60 mg, 0.077 mmol) in THF (3 mL). The reaction mixture was stirred at 40° C. for 16 h. The reaction mixture was concentrated to the aqueous layer under reduced pressure. This aqueous layer was acidified with aqueous HCl (2 M) to pH=3. The solid was collected by filtration and was purified by preparative HPLC (Column: XBridge Prep OBD C18 Column, 30×150 mm 5 um; Mobile Phase A: Water (50 mmol/L NH₄HCO₃), Mobile Phase B: ACN; Gradient: 43% B to 63% B in 7 min) to afford Compound 17 (24.2 mg, yield: 40%) as a white solid.

¹H NMR (300 MHz, Chloroform-d) δ ppm 8.32 (m, 1H), 7.67 (d, J=89 Hz, 1H), 7.34-7.08 (m, 4H), 5.50 (d, J=30 Hz, 2H), 3.91-3.74 (m, 2H), 3.70-3.43 (m, 14H), 3.39 (s, 3H), 3.23-3.07 (m, 5H), 3.06-2.78 (m, 811), 2.32 (s, 2H).

¹⁹F NMR (282 MHz, Chloroform-d) δ ppm −114.633.

### Compound 18

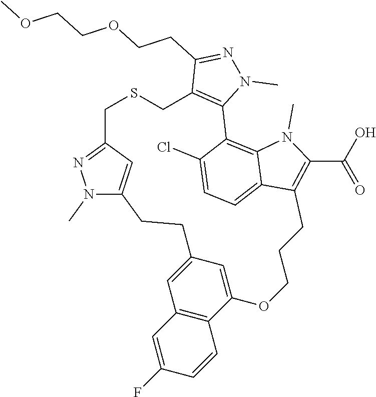 [SMILES: COCCOCCc1nn(C)c2c1CSCc1cc(n(C)n1)CCc1cc(c3ccc(F)cc3c1)OCCCc1c(C(=O)O)n(C)c3c-2c(Cl)ccc13] [0597] Sₐ or Rₐ; one atropisomer but absolute stereochemistry undetermined

Compound 18 was prepared according to an analogous procedure as for Compound 17, starting from Intermediate 100 instead of Intermediate 99.

¹H NMR (300 MHz, Chloroform-d) δ ppm 8.29 (s, 1H), 7.59 (d, J=9 Hz, 1H), 7.33 (d, J=9 Hz, 1H), 7.25-7.08 (m, 2H), 6.98 (d, J=6 Hz, 1H), 5.67 (s, 1H), 5.21 (s, 1H), 3.91-3.71 (m, 3H), 3.64 (s, 3H), 3.53 (d, J=15 Hz 6H), 3.46-3.34 (m, 4H), 3.30 (s, 3H), 3.25 (s, 5H), 3.14-2.93 (m, 3H), 2.92-2.67 (m, 5H), 2.30 (s, 2H).

¹⁹F NMR (282 MHz, Chloroform-d) δ ppm −114.726.

### Compound 19

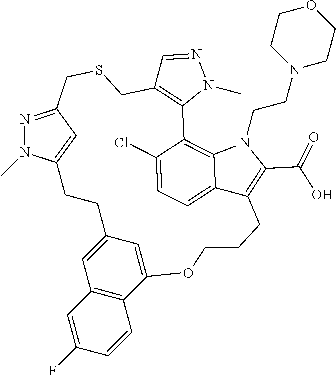 [SMILES: Cn1nc2cc1CCc1cc(c3ccc(F)cc3c1)OCCCc1c(C(=O)O)n(CCN3CCOCC3)c3c(c(Cl)ccc13)-c1c(cnn1C)CSC2] [0602] Rₐ or Sₐ; one atropisomer but absolute stereochemistry undetermined

LiOH (6.8 mg, 0.285 mmol, 10 eq.) was added to a solution of Intermediate 108 (22 mg, 0.028 mmol) in water (0.5 mL), MeOH (1 mL), and THF (1 mL) and the reaction mixture was stirred for 16 h at 55° C. The solvents were evaporated and water (2 mL) was added. Aqueous HCl (1 N) was added to reach pH=5-6. The water layer was extracted with EtOAc (3×5 mL) and the combined organic layer was dried with MgSO₄, filtered, and concentrated in vacuo. The residue was purified by preparative SFC (Stationary phase: Chiralcel Diacel OJ 20×250 mm, Mobile phase: CO₂, EtOH+0.4% iPrNH₂) to afford Compound 19 (9.1 mg, yield: 42%).

1H NMR (400 MHz, Chloroform-d, 27° C.) δ ppm 2.30-2.43 (m, 2H) 2.43-2.51 (m, 2H) 2.55-2.75 (m, 4H) 2.80 (br d, J=9.5 Hz, 2H) 2.87-2.98 (m, 3H) 3.09-3.20 (m, 2H) 3.26 (d, J=3.2 Hz, 1H) 3.35 (d, J=3.3 Hz, 1H) 3.46-3.56 (m, 1H) 3.52 (s, 3H) 3.57-3.68 (m, 2H) 3.65 (s, 3H) 3.71-3.82 (m, 4H) 3.89-3.99 (m, 1H) 4.34-4.48 (m, 2H) 5.08 (s, 1H) 5.91 (s, 1H) 6.98 (br d, J=8.5 Hz, 1H) 7.05 (s, 1H) 7.20 (td, J=8.7, 2.5 Hz, 1H) 7.31 (dd, J=10.1, 2.5 Hz, 1H) 7.59 (d, J=7.6 Hz, 1H) 7.59 (s, 1H) 8.27 (dd, J=9.0, 5.8 Hz, 1H).

### Compound 20

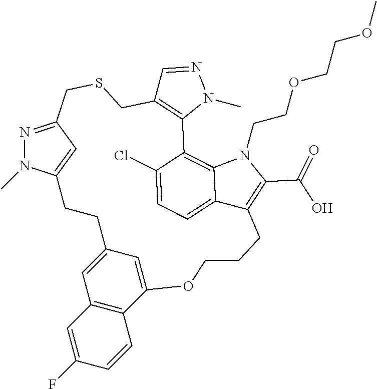 [0606] Rₐ or Sₐ; one atropisomer but absolute stereochemistry undetermined

LiOH (4 mg, 0.166 mmol, 10 eq.) was added to a solution of Intermediate 109 (14 mg, 0.017 mmol) in MeOH (0.7 mL), water (0.3 mL), and THE (0.7 mL) and the solution was stirred for 1 h at 60° C. The solvents were evaporated and the residue was purified by preparative SFC (Stationary phase: Chiralcel Diacel IH 20×250 mm, Mobile phase: CO₂, EtOH+0.4% iPrNH₂) to afford Compound 20 (10.8 mg, yield: 87%).

¹H NMR (400 MHz, Chloroform-d, 51° C.) δ ppm 2.11-2.27 (m, 1H) 2.30-2.42 (m, 1H) 2.62-2.76 (m, 2H) 2.80-2.94 (m, 3H) 3.08-3.19 (m, 3H) 3.19-3.26 (m, 11H) 3.21 (s, 3H) 3.31-3.40 (m, 3H) 3.41 (s, 3H) 3.43-3.50 (m, 3H) 3.58 (s, 3H) 3.61-3.68 (m, 2H) 3.80 (br s, 1H) 4.11-4.25 (m, 1H) 4.71 (br s, 1H) 5.00 (br s, 1H) 5.89 (s, 1H) 6.88 (d, J=8.6 Hz, 1H) 7.04 (s, 1H) 7.15 (td, J=8.7, 2.4 Hz, 1H) 7.30 (dd, J=10.1, 2.6 Hz, 1H) 7.53 (t, J=4.3 Hz, 2H) 8.27 (dd, J=9.2, 5.7 Hz, 1H).

### Compound 21

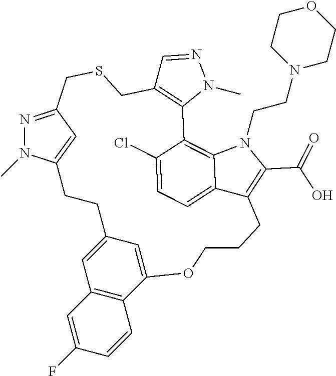 [SMILES: Cn1nc2cc1CCc1cc(c3ccc(F)cc3c1)OCCCc1c(C(=O)O)n(CCN3CCOCC3)c3c(c(Cl)ccc13)-c1c(cnn1C)CSC2] [0610] Sₐ or Rₐ; one atropisomer but absolute stereochemistry undetermined

LiOH (6.2 mg, 0.259 mmol, 10 eq.) was added to a solution of Intermediate 110 (20 mg, 0.026 mmol) in THF (1 mL), water (0.5 mL), and MeOH (1 mL) and the reaction mixture was stirred at 60° C. for 3 h. The solvents were evaporated and the residue was purified by preparative HPLC (Stationary phase: RP XBridge Prep C18 OBD—5 μm, 50×250 mm, Mobile phase: 0.25% NH₄HCO₃ solution in water, CH₃CN) to afford Compound 21 (9.6 mg, yield: 49%)

¹H NMR (400 MHz, Chloroform-d, 27° C.) δ ppm 2.33-2.48 (m, 4H) 2.50-2.63 (m, 2H) 2.65-2.73 (m, 1H) 2.75-2.86 (m, 21H) 2.89-3.00 (m, 31H) 3.08-3.18 (m, 2H) 3.23 (d, J=14.5 Hz, 1H) 3.36 (d, J=15.0 Hz, 1H) 3.44-3.57 (m, 1H) 3.52 (s, 3H) 3.58-3.68 (m, 2H) 3.63-3.66 (m, 3H) 3.73 (br s, 4H) 3.87-4.01 (m, 1H) 4.37 (br s, 2H) 5.11 (s, 1H) 5.92 (s, 1H) 7.00 (d, J=9.2 Hz, 1H) 7.04 (s, 1H) 7.18 (td, J=8.9, 2.3 Hz, 1H) 7.30 (dd, J=10.2, 2.5 Hz, 1H) 7.58 (s, 1H) 7.62 (br d, J=8.8 Hz, 1H) 8.25 (dd, J=8.9, 6.1 Hz, 1H).

### Compound 22

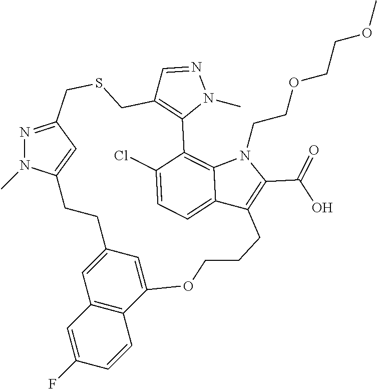 [0614] Sₐ or Rₐ; one atropisomer but absolute stereochemistry undetermined

Compound 22 was prepared according to an analogous procedure as for Compound 21, starting from Intermediate 111 instead of Intermediate 110.

¹H NMR (400 MHz, Chloroform-d, 51° C.) δ ppm 2.26-2.41 (m, 2H) 2.87 (br s, 4H) 3.00 (d, J=13.0 Hz, 1H) 3.10 (d, J=12.8 Hz, 1H) 3.14-3.22 (m, 2H) 3.24 (s, 3H) 3.34-3.43 (m, 10H) 3.59-3.78 (m, 2H) 3.67 (s, 3H) 3.94-4.08 (m, 1H) 4.54 (dt, J=15.6, 5.2 Hz, 1H) 5.35 (s, 1H) 5.81 (s, 1H) 7.07 (s, 1H) 7.09 (d, J=8.6 Hz, 1H) 7.20 (td, J=8.6, 2.4 Hz, 1H) 7.31 (dd, J=10.0, 2.5 Hz, 2H) 7.56 (s, 1H) 7.66 (d, J=8.6 Hz, 1H) 8.28 (dd, J=9.2, 5.7 Hz, 1H).

### Compound 23 and Compound 24

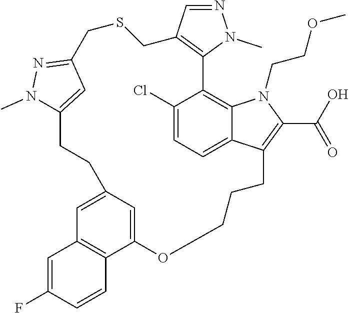 [0618] Compound 23: Rₐ or Sₐ; one atropisomer but absolute stereochemistry undetermined
Compound 24: Sₐ or Rₐ; one atropisomer but absolute stereochemistry undetermined

NaH (60% dispersion in mineral oil, 13.2 mg, 0.331 mmol, 3 eq.) was added to a solution of Intermediate 118 (76 mg, 0.11 mmol) in dry THF (1 mL) cooled to 0° C. under nitrogen atmosphere. The reaction mixture was stirred at 0° C. for 5 min before MeI (0.027 mL, 0.442 mmol, 4 eq.) was added. The reaction mixture was then stirred at room temperature for 16 h. The reaction mixture was diluted with water and extracted with EtOAc. The water layer was acidified with aqueous HCl (1 M), to reach pH=4-5. This mixture was extracted with EtOAc and the combined organic layer was evaporated. The residue was purified by preparative SFC (Stationary phase: Chiralpak Diacel AD 20×250 mm, Mobile phase: CO₂, EtOH-iPrOH (50-50)+0.4% iPrNH₂) to give Compound 23 (14.6 mg, yield: 19%) and Compound 24 (18.8 mg, yield: 24%).

Compound 23: ¹H NMR (400 MHz, Chloroform-d, 27° C.) δ ppm 2.24-2.35 (m, 2H) 2.80-2.93 (m, 41H) 2.97 (s, 21H) 3.10 (br d, J=14.5 Hz, 1H) 3.11 (s, 3H) 3.18-3.24 (m, 1H) 3.27 (s, 3H) 3.31 (t, J=5.3 Hz, 2H) 3.48 (br d, J=15.0 Hz, 1H) 3.55-3.70 (m, 4H) 3.69 (s, 3H) 3.93 (dt, J=15.3, 5.6 Hz, 1H) 4.51 (dt, J=15.1, 4.9 Hz, 1H) 5.41 (s, 1H) 5.66 (s, 1H) 7.09 (s, 1H) 7.13 (d, J=8.6 Hz, 1H) 7.17-7.24 (m, 1H) 7.31 (dd, J=10.0, 2.5 Hz, 1H) 7.56 (s, 1H) 7.69 (d, J=8.6 Hz, 1H) 8.27 (dd, J=9.0, 5.9 Hz, 1H).

Compound 24: ¹H NMR (400 MHz, Chloroform-d, 27° C.) δ ppm 2.23-2.33 (m, 2H) 2.85 (s, 4H) 2.94 (br d, J=13.2 Hz, 1H) 3.04 (dd, J=13.4, 1.0 Hz, 1H) 3.08 (s, 3H) 3.13 (d, J=14.7 Hz, 1H) 3.18 (br d, J=9.5 Hz, 1H) 3.23-3.33 (m, 2H) 3.41 (d, J=15.0 Hz, 1H) 3.36 (s, 3H) 3.56-3.75 (m, 4H) 3.66 (s, 3H) 3.88-3.99 (m, 1H) 4.54 (dt, J=15.1, 4.9 Hz, 1H) 5.30 (s, 1H) 5.76 (s, 1H) 7.05 (d, J=6.6 Hz, 1H) 7.06 (s, 1H) 7.19 (td, J=8.7, 2.8 Hz, 1H) 7.30 (dd, J=9.9, 2.4 Hz, 1H) 7.55 (s, 1H) 7.63 (d, J=8.6 Hz, 1H) 8.26 (dd, J=9.1, 5.8 Hz, 1H).

### Compound 25

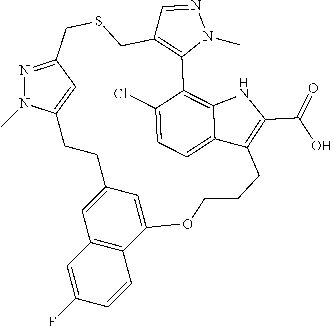 [SMILES: Cn1nc2cc1CCc1cc(c3ccc(F)cc3c1)OCCCc1c(C(=O)O)[nH]c3c(c(Cl)ccc13)-c1c(cnn1C)CSC2] [0624] Rₐ or Sₐ; one atropisomer but absolute stereochemistry undetermined

LiOH (3.6 mg, 0.152 mmol, 10 eq.) was added to a solution of Intermediate 106 (10 mg, 0.0152 mmol) in THF (0.6 mL), MeOH (0.6 mL), and water (0.3 mL) and the reaction mixture was stirred at 60° C. for 2 h. The solvents were evaporated and the residue was purified by column chromatography on silica gel (Reveleris column 4 g; DCM/MeOH 100/0 to 90/10) to give Compound 25 (4.5 mg, yield: 46%) as a white solid.

NMR: ¹H NMR (400 MHz, Chloroform-d, 27° C.) δ ppm 2.25-2.46 (m, 2H) 2.64 (d, J=13.2 Hz, 1H) 2.67-2.78 (m, 1H) 2.78-2.89 (m, 1H) 2.90-3.02 (m, 2H) 3.11 (s, 3H) 3.16 (d, J=13.4 Hz, 1H) 3.25 (ddd, J=13.4, 8.7, 4.5 Hz, 1H) 3.40-3.48 (m, 2H) 3.51 (d, J=15.0 Hz, 1H) 3.59 (s, 3H) 3.64-3.77 (m, 2H) 5.34 (s, 1H) 5.76 (s, 1H) 7.07 (d, J=8.8 Hz, 1H) 7.16 (s, 1H) 7.20-7.24 (m, 1H) 7.33-7.38 (m, 1H) 7.36 (s, 1H) 7.63 (d, J=8.6 Hz, 1H) 8.35 (dd, J=9.2, 5.9 Hz, 1H) 10.45 (br s, 1H).

### Compound 26

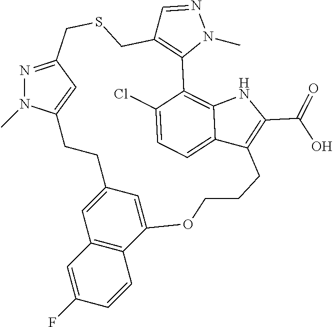 [SMILES: Cn1nc2cc1CCc1cc(c3ccc(F)cc3c1)OCCCc1c(C(=O)O)[nH]c3c(c(Cl)ccc13)-c1c(cnn1C)CSC2] [0628] Sₐ or Rₐ; one atropisomer but absolute stereochemistry undetermined

Compound 26 was prepared according to an analogous procedure as for Compound 25, starting from Intermediate 107 instead of Intermediate 106.

NMR: ¹H NMR (400 MHz, Chloroform-d, 27° C.) δ ppm 2.28-2.46 (m, 2H) 2.65 (d, J=13.2 Hz, 1H) 2.68-2.78 (m, 1H) 2.80-2.89 (m, 1H) 2.91-3.03 (m, 2H) 3.12 (s, 3H) 3.17 (d, J=13.2 Hz, 1H) 3.21-3.31 (m, 1H) 3.39-3.47 (m, 2H) 3.48-3.55 (m, 1H) 3.60 (s, 3H) 3.63-3.77 (m, 2H) 5.34 (s, 1H) 5.77 (s, 1H) 7.08 (d, J=8.6 Hz, 1H) 7.16 (s, 1H) 7.21-7.25 (m, 1H) 7.36 (dd, J=10.2, 2.5 Hz, 1H) 7.38 (s, 1H) 7.63 (d, J=8.6 Hz, 1H) 8.36 (dd, J=9.1, 5.8 Hz, 1H) 10.44 (br s, 1H).

### Compound 27

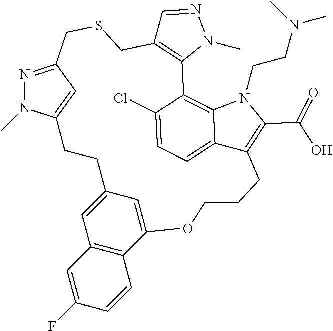 [0632] Sₐ or Rₐ; one atropisomer but absolute stereochemistry undetermined

LiOH (13 mg, 0.548 mmol, 10 eq.) was added to a solution of Intermediate 123 (40 mg, 0.0548 mmol) in THF (2 mL), MeOH (2 mL), and water (1 mL) and the reaction mixture was stirred at 55° C. for 2 h. The solvents were evaporated and the residue was purified by preparative HPLC (Stationary phase: RP XBridge Prep C18 OBD-10 μm, 30×150 mm, Mobile phase: 0.25% NH₄HCO₃ solution in water, CH₃CN) to give Compound 27 (3 mg, yield: 8%).

¹H NMR (600 MHz, Chloroform-d, 27° C.) δ ppm 2.34 (ddd, J=14.4, 9.7, 5.0 Hz, 1H) 2.43 (br s, 1H) 2.43 (s, 6H) 2.73-2.79 (m, 2H) 2.81 (br d, J=13.7 Hz, 1H) 2.90-2.96 (m, 2H) 2.91-2.94 (m, 1H) 3.11-3.17 (m, 3H) 3.32 (d, J=14.5 Hz, 1H) 3.40 (d, J=14.5 Hz, 1H) 3.47 (s, 3H) 3.57-3.61 (m, 1H) 3.62 (s, 3H) 3.65-3.70 (m, 1H) 3.85-3.91 (m, 1H) 4.38 (br s, 1H) 4.71 (br s, 1H) 5.06 (s, 1H) 5.90 (s, 1H) 6.91 (d, J=8.4 Hz, 1H) 7.06 (s, 1H) 7.21 (td, J=8.8, 2.6 Hz, 1H) 7.32 (dd, J=9.9, 2.5 Hz, 1H) 7.58 (s, 1H) 7.59 (d, J=7.4 Hz, 1H) 8.33 (dd, J=9.2, 5.8 Hz, 1H).

### Analytical Analysis

### LCMS Methods

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl), the reported value is the one obtained for the lowest isotope mass. All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica.

LCMS Method Codes (Flow expressed in mL/min; column temperature (T) in ° C.; Run time in minutes)

| Method | | | | | Flow/ | Run |
|---|---|---|---|---|---|---|
| Code | Instrument | column | mobile phase | gradient | Col T | time |
| 1 | Agilent | Waters | A: water with | 100% A held for 1 min. | 0.8/ | 10 |
| | 1200 | XB ridge | 0.05% NH₃•H₂O; | Then to 40% A and 60% | 40 | |
| | equiped | ShieldRP 18 | B: CH₃CN) | B in 4 min | | |
| | with MSD | column (5 m, | | Then to 5% A and 95% | | |
| | 6110 or | 2.1 × 50 mm) | | B in 2.5 min. | | |
| | equivalent | | | Return to 100% | | |
| | | | | A in 2 min and | | |
| | | | | hold for 0.5 min | | |
| 2 | Agilent | Waters | A: water with | 90% A held for 0.8 min. | 0.8/ | 10 |
| | 1200 | Xbridge-C18 | 0.04% TFA | Then to 20% A and 80% | 50 | |
| | equiped | column (5 μm, | B: CH₃CN with | B in 3.7 minutes, | | |
| | with MSD | 2.0 × 50 mm) | 0.02% TFA | held for 3 min. | | |
| | 6110 or | | | Return to 90% A | | |
| | equivalent | | | in 2 min and hold | | |
| | | | | for 0.5 min. | | |
| 3 | Waters: | Waters: | A: 10 mM CH₃COONH₄ | From 100% A to 5% A | 0.7/ | 3.5 |
| | Acquity ® | BEH (1.8 μm, | in 95% H₂O + | in 2.1 min, | 55 | |
| | UPLC ® - | 2.1 × 100 mm) | 5% CH₃CN | to 0% A in 0.9 min, | | |
| | DAD and SQD | | B: CH₃CN | to 5% A in 0.5 min | | |
| 4 | Waters: | Waters: | A: 10 mM CH₃COONH₄ | From 100% A to 5% A | 0.6/ | 3.5 |
| | Acquity ® | BEH (1.8 μm, | in 95% H₂O + | in 2.1 min, | 55 | |
| | UPLC ® - | 2.1 × 100 mm) | 5% CH₃CN | to 0% A in 0.9 min, | | |
| | DAD and SQD | | B: CH₃CN | to 5% A in 0.5 min | | |
| 5 | Waters: | Waters: | A: 10 mM CH₃COONH₄ | From 95% A to 5% A | 0.8/ | 2 |
| | Acquity ® | BEH C18 (1.7 μm, | in 95% H₂O + | in 1.3 min, | 55 | |
| | UPLC ® - | 2.1 × 50 mm) | 5% CH₃CN | held for 0.7 min | | |
| | DAD and SQD | | B: CH₃CN | | | |
| 6 | Shimadzu | HALO 90 A | A: Water/0.05% TFA | 30% B to 95% B | 1.5/ | 3 |
| | LCMS- | C18, 3.0 * | B: ACN/0.05% TFA | in 2 min, hold | 40 | |
| | 2020 | 30 mm, 2.0 μm | | 0.7 min at 95% B | | |
| 7 | Waters: | Waters: | A: 10 mM CH₃COONH₄ | From 100% A to 5% A | 0.6/ | 3.5 |
| | Acquity ® | BEH (1.8 μm, | in 95% H₂O + | in 2.10 min, to 0% A | 55 | |
| | UPLC ® - | 2.1*100 mm) | 5% CH₃CN | in 0.90 min, to 5% | | |
| | DAD and SQD | | B: CH₃CN | A in 0.5 min | | |
| 8 | Waters: | Waters: | A: 0.1% NH₄HCO₃ | From 100% A to 5% | 0.6/ | 4.5 |
| | Acquity ® | BEH (1.8 μm, | in 95% H₂O + | A in 2.10 min, | 55 | |
| | UPLC ® - | 2.1 * 100 mm) | 5% CH3CN | to 0% A in 0.9 min, | | |
| | DAD and SQD | | B: CH₃CN | to 5% A in 0.5 min | | |

### LCMS Results (RT Means Retention Time)

| Co. No. | LCMS results |
|---|---|
| 1 | confirms the MW (RT: 3.68, [M + H]+ 672, LCMS Method 1) |
| 2 | confirms the MW (RT: 4.14, [M + H]+ 672, LCMS Method 2) |
| 3 | confirms the MW (RT: 1.79, [M + H]+ 728, LCMS Method 3) |
| 4 | confirms the MW (RT: 1.79, [M + H]+ 728, LCMS Method 3) |
| 5 | confirms the MW (RT: 4.05, [M + H]+ 659, LCMS Method 2) |
| 6 | confirms the MW (RT: 4.05, [M + H]+ 659, LCMS Method 2) |
| 7 | confirms the MW (RT: 1.86, [M + H]+ 746, LCMS Method 4) |
| 8 | confirms the MW (RT: 1.81, [M + H]+ 746, LCMS Method 4) |
| 9 | confirms the MW (RT: 4.17, [M + H]+ 672, LCMS Method 2) |
| 10 | confirms the MW (RT: 4.17, [M + H]+ 672, LCMS Method 2) |
| 10a | confirms the MW (RT: 1.85, [M + H]+ 672, LCMS Method 4) |
| 11 | confirms the MW (RT: 4.13, [M + H]+ 654, LCMS Method 2) |
| 11a | confirms the MW (RT: 1.82, [M + H]+ 654, LCMS Method 4) |
| 12 | confirms the MW (RT: 4.13, [M + H]+ 654, LCMS Method 2) |
| 12a | confirms the MW (RT: 1.84, [M + H]+ 654, LCMS Method 4) |
| 13 | confirms the MW (RT: 0.96, [M + H]+ 654, LCMS Method 5) |
| 14 | confirms the MW (RT: 0.96, [M + H]+ 654, LCMS Method 5) |
| 15 | confirms the MW (RT: 1.89, [M + H]+ 764, LCMS Method 4) |
| 16 | confirms the MW (RT: 1.84, [M + H]+ 764, LCMS Method 4) |
| 17 | confirms the MW (RT: 1.31, [M + H]+ 760, LCMS Method 6) |
| 18 | confirms the MW (RT: 1.31, [M + H]+ 760, LCMS Method 6) |
| 19 | confirms the MW (RT: 1.90, [M + H]+ 757, LCMS Method 7) |
| 20 | confirms the MW (RT: 1.80, [M + H]+ 746, LCMS Method 4) |
| 21 | confirms the MW (RT: 1.73, [M + H]+ 757, LCMS Method 4) |
| 22 | confirms the MW (RT: 1.74, [M + H]+ 746, LCMS Method 4) |
| 23 | confirms the MW (RT: 1.78, [M + H]+ 702, LCMS Method 7) |
| 24 | confirms the MW (RT: 1.78, [M + H]+ 702, LCMS Method 7) |
| 25 | confirms the MW (RT: 1.00, [M + H]+ 645, LCMS Method 5) |
| 26 | confirms the MW (RT: 1.00, [M + H]+ 645, LCMS Method 5) |
| 27 | confirms the MW (RT: 1.83, [M + H]+ 715, LCMS Method 8) |

### SFC-MS Methods

The SFC measurement was performed using an Analytical Supercritical fluid chromatography (SFC) system composed by a binary pump for delivering carbon dioxide (CO2) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software. Analytical SFC-MS Methods (Flow expressed in mL/min; column temperature (Col T) in C; Run time in minutes, Backpressure (BPR) in bars. “iPrNH₂” means isopropylamine, “iPrOH” means 2-propanol, “DEA” means diethylamine, “EtOH” means ethanol, “m” mean minutes.

### SFC Methods:

| | | | | | Run |
|---|---|---|---|---|---|
| SFC | | | | Flow | time |
| Method | Column | mobile phase | gradient | Col T | BPR |
| 1 | Brand | A: CO₂ | 5% B to 40% in 2 min | 4 | 4 |
| | Chiralpak ® AD-3 | B: EtOH + 0.05% | 40% B hold 1.2 min | 35 | 100 |
| | column (3.0 μm, | DEA | 5% B hold 0.8 min | | |
| | 50 × 4.6 mm) | | | | |
| 2 | Brand | A: CO₂ | 5% B to 40% in 5 min | 2.5 | 7 |
| | Chiralcel ® OJ-3 | B: EtOH + 0.05% | 40% B to 5% in 0.5 min | 35 | 100 |
| | column (3.0 μm, | DEA | 5% B hold 1.5 min | | |
| | 150 × 4.6 mm) | | | | |
| 3 | Brand | A: CO₂ | 5% B to 40% in 2 min | 4 | 4 |
| | Chiralpak ® OD-3 | B: EtOH + 0.05% | and 40% B hold 1.2 min, | 35 | 100 |
| | column (3.0 μm, | DEA | 5% B hold 0.8 min | | |
| | 50 × 4.6 mm) | | | | |
| 4 | Brand | A: CO₂ | 5% B to 40% in 4.5 min | 2.8 | 8 |
| | Chiralcel ®OD-3 | B: EtOH + 0.05% | and 40% B hold 2.5 min, | 40 | 100 |
| | column (3.0 μm, | DEA | 5% B hold 1 min | | |
| | 50 × 4.6 mm) | | | | |
| 5 | Daicel | A: CO₂ | 10%-50% B in 6 min, | 2.5 | 9.5 |
| | Chiralpak ® IH3 | B: EtOH + 0.2% | hold 3.5 min | 40 | 130 |
| | column (3.0 μm, | iPrNH₂ | | | |
| | 150 × 4.6 mm) | | | | |
| 6 | Daicel | A: CO₂ | 10% to 50% B in | 2.0 | 3.0 |
| | Chiralpak ® IH3 | B: iPrOH + 0.1% | 2 min, hold 1 min | 35 | 103 |
| | column (3.0 * 50 | Et₂NH | | | |
| | mm, 3 μm) | | | | |
| 7 | Daicel | A: CO₂ | 10%-50% B in | 2.5 | 9.5 |
| | Chiralpak ® AD-H | B: EtOH-iPrOH + 0.2% | 6 min, hold 3.5 min | 40 | 110 |
| | column (3.0 μm, | iPrNH₂ | | | |
| | 150 × 4.6 mm) | | | | |
| 8 | Daicel | A: CO₂ | 10%-50% B in | 2.5 | 9.5 |
| | Chiralpak ® AD-H | B: EtOH + 0.2% | 6 min, hold 3.5 min | 40 | 110 |
| | column (3.0 μm, | iPrNH₂ | | | |
| | 150 × 4.6 mm) | | | | |

TABLE
Analytical SFC data - Rₜ means retention time (in minutes),
[M + H]⁺ means the protonated mass of the
compound, method refers to the method used for (SFC)MS
analysis of enantiomerically pure compounds. No. means number.

| Co. No. | SFC Method | Rt | [M + H]⁺ |
|---|---|---|---|
| 3 | 1 | 1.77 | 672 |
| 4 | 1 | 1.82 | 672 |
| 7 | 2 | 5.42 | 658 |
| 8 | 2 | 4.58 | 658 |
| 9 | 3 | 1.90 | 672 |
| 10 | 3 | 1.82 | 672 |
| 10a | 8 | 4.13 | 672 |
| 11 | 4 | 4.47 | 654 |
| 12 | 4 | 4.22 | 654 |
| 15 | 5 | 3.73 | 764 |
| 16 | 5 | 4.20 | 764 |
| 17 | 6 | 1.89 | 760 |
| 18 | 6 | 1.68 | 760 |
| 23 | 7 | 4.95 | 702 |
| 24 | 7 | 5.81 | 702 |

### NMR

¹H NMR and ¹⁹F NMR spectra were recorded on Bruker Avance III 400 MHz and Avance NEO 400 MHz spectrometers. CDCl₃ was used as solvent, unless otherwise mentioned. The chemical shifts are expressed in ppm relative to tetramethylsilane.

### Pharmacological Analysis

### Biological Example 1

Terbium labeled Myeloid Cell Leukemia 1 (Mcl-1) homogeneous time-resolved fluorescence (HTRF) binding assay utilizing the BIM BH3 peptide (H2N-(C/Cy5Mal) WIAQELRRIGDEFN-OH) as the binding partner for Mcl-1.

Apoptosis, or programmed cell death, ensures normal tissue homeostasis, and its dysregulation can lead to several human pathologies, including cancer. Whilst the extrinsic apoptosis pathway is initiated through the activation of cell-surface receptors, the intrinsic apoptosis pathway occurs at the mitochondrial outer membrane and is governed by the binding interactions between pro- and anti-apoptotic Bcl-2 family proteins, including Mcl-1. In many cancers, the anti-apoptotic Bcl-2 protein(s), such as the Mcl-1, are upregulated, and in this way the cancer cells can evade apoptosis. Thus, inhibition of the Bcl-2 protein(s), such as Mcl-1, may lead to apoptosis in cancer cells, providing a method for the treatment of said cancers.

This assay evaluated inhibition of the BH3 domain: Mcl-1 interaction by measuring the displacement of Cy5-labeled BIM BH3 peptide (H₂N—(C/Cy5Mal) WIAQELRRIGDEFN-OH) in the HTRF assay format.

### Assay Procedure

The following assay and stock buffers were prepared for use in the assay: (a) Stock buffer: 10 mM Tris-HCl, pH=7.5+150 mM NaCl, filtered, sterilized, and stored at 4° C.; and (b) 1×assay buffer, where the following ingredients were added fresh to stock buffer: 2 mM dithiothreitol (DTT), 0.0025% Tween-20, 0.1 mg/mL bovine serum albumin (BSA). The 1×Tb-Mcl-1+Cy5 Bim peptide solution was prepared by diluting the protein stock solution using the 1×assay buffer (b) to 25 pM Tb-Mcl-1 and 8 nM Cy5 Bim peptide.

Using the Acoustic ECHO, 100 nL of 100×test compound(s) were dispensed into individual wells of a white 384-well Perkin Elmer Proxiplate, for a final compound concentration of 1× and final DMSO concentration of 1%. Inhibitor control and neutral control (NC, 100 nL of 100% DMSO) were stamped into columns 23 and 24 of assay plate, respectively. Into each well of the plate was then dispensed 10 μL of the 1×Tb-Mcl-1+Cy5 Bim peptide solution. The plate was centrifuged with a cover plate at 1000 rpm for 1 minute, then incubated for 60 minutes at room temperature with plates covered.

The TR-FRET signal was read on an BMG PHERAStar FSX MicroPlate Reader at room temperature using the HTRF optic module (HTRF: excitation: 337 nm, light source: laser, emission A: 665 nm, emission B: 620 nm, integration start: 60 μs, integration time: 400 μs).

### Data Analysis

The BMG PHERAStar FSX MicroPlate Reader was used to measure fluorescence intensity at two emission wavelengths—665 nm and 620 nm—and report relative fluorescence units (RFU) for both emissions, as well as a ratio of the emissions (665 nm/620 nm)*10,000. The RFU values were normalized to percent inhibition as follows:

% inhibition=(((NC−IC)−(compound−IC))/(NC−IC))*100

where IC (inhibitor control, low signal)=mean signal of 1×Tb-MCl-1+Cy5 Bim peptide+inhibitor control or 100% inhibition of Mcl-1; NC (neutral control, high signal)=mean signal 1×Tb-MCl-1+Cy5 Bim peptide with DMSO only or 0% inhibition

An 11-point dose response curve was generated to determine IC₅₀ values (using GenData) based on the following equation:

Y=Bottom+(Top−Bottom)/(1+10\hat{ }((log IC₅₀−X)*HillSlope))

where Y=% inhibition in the presence of X inhibitor concentration; Top=100% inhibition derived from the IC (mean signal of Mcl-1+inhibitor control); Bottom=0% inhibition derived from the NC (mean signal of Mcl-1+DMSO); Hillslope=Hill coefficient; and IC₅₀=concentration of compound with 50% inhibition in relation to top/neutral control (NC).

Kᵢ=IC₅₀/(1+[L]/Kd)

In this assay [L]=8 nM and Kd=10 nM

Representative compounds of the present invention were tested according to the procedure as described above, with results as listed in the Table below (n.d. means not determined).

| Compound | Tb-MCL1 Kᵢ (nM) |
|---|---|
| 1 | 0.746 |
| 2 | 0.039 |
| 3 | 98.25 |
| 4 | 0.023 |
| 5 | 9.54 |
| 6 | 0.062 |
| 7 | 52.26 |
| 8 | 0.025 |
| 9 | 18.66 |
| 10 | 0.307 |
| 10a | 0.041 |
| 11 | 3.02 |
| 11a | 3.68 |
| 12 | n.d. |
| 12a | 0.031 |
| 13 | 0.051 |
| 14 | 6.15 |
| 15 | 2.58 |
| 16 | 0.021 |
| 17 | 0.011 |
| 18 | 2.1 |
| 19 | 50.1 |
| 20 | 199.5 |
| 21 | 0.020 |
| 22 | 0.029 |
| 23 | 0.024 |
| 24 | 2.15 |
| 25 | 0.807 |
| 26 | 0.045 |
| 27 | 0.031 |

### Biological Example 2

MCL-1 is a regulator of apoptosis and is highly over-expressed in tumor cells that escape cell death. The assay evaluates the cellular potency of small-molecule compounds targeting regulators of the apoptosis pathway, primarily MCL-1, Bfl-1, Bcl-2, and other proteins of the Bcl-2 family. Protein-protein inhibitors disrupting the interaction of anti-apoptotic regulators with BH3-domain proteins initiate apoptosis.

The Caspase-Glo® 3/7 Assay is a luminescent assay that measures caspase-3 and -7 activities in purified enzyme preparations or cultures of adherent or suspension cells. The assay provides a proluminescent caspase-3/7 substrate, which contains the tetrapeptide sequence DEVD. This substrate is cleaved to release aminoluciferin, a substrate of luciferase used in the production of light. Addition of the single Caspase-Glo® 3/7 Reagent in an “add-mix-measure” format results in cell lysis, followed by caspase cleavage of the substrate and generation of a “glow-type” luminescent signal.

This assay uses the MOLP-8 human multiple myeloma cell line, which is sensitive to MCL-1 inhibition.

### Materials:

Perkin Elmer Envision
Multidrop 384 and small volume dispensing cassettes
Centrifuge
Countess automated cell counter
Countess counting chamber slides
Assay plate: ProxiPlate-384 Plus, White 384-shallow well Microplate
Sealing tape: Topseal A plus
T175 culture flask

| | | |
|---|---|---|
| Product | Units | Storage |

| | | | | |
|---|---|---|---|---|
| RPMI1640 (no L-Glutamine, | 500 | mL | 4° | C. |
| no phenol red) | | | | |
| Foetal Bovine Serum (FBS) | 500 | mL | 4° | C. |
| (Heat inactivated) | | | | |
| L-Glutamine (200 mM) | 100 | ml | −20° | C. |
| Gentamicin (50 mg/mL) | 100 | mL | 4° | C. |
| Caspase 3/7 Detection kit | 100 | mL | −20° | C. |

10 × 10 mL

### Cell Culture Media:

| MOLP8 | | |
|---|---|---|
| RPMI-1640 medium | 500 | mL |
| 20% FBS (heat inactivated) | 120 | mL |
| 2 mM L-Glutamine | 6.2 | mL |
| 50 μg/mL Gentamicin | 620 | μL |
| Assay media | | |
| RPMI-1640 medium | 500 | mL |
| 10% FBS (Heat inactivated) | 57 | mL |
| 2 mM L-Glutamine | 5.7 | mL |
| 50 μg/mL Gentamicin | 570 | μL |

### Cell Culture:

Cell cultures were maintained between 0.2 and 2.0×10⁶ cells/mL. The cells were harvested by collection in 50 mL conical tubes. The cells were then pelleted at 500 g for 5 mins before removing supernatant and resuspension in fresh pre-warmed culture medium. The cells were counted and diluted as needed.

### Caspase-Glo Reagent:

The assay reagent was prepared by transferring the buffer solution to the substrate vial and mixing. The solution may be stored for up to 1 week at 4° C. with negligible loss of signal.

### Assay Procedure:

Compounds were delivered in assay-ready plates (Proxiplate) and stored at −20° C.

Assays always include 1 reference compound plate containing reference compounds. The plates were spotted with 40 nL of compounds (0.5% DMSO final in cells; serial dilution; 30 μM highest conc. 1/3 dilution, 10 doses, duplicates). The compounds were used at room temperature and 4 μL of pre-warmed media was added to all wells except column 2 and 23. The negative control was prepared by adding 1% DMSO in media. The positive control was prepared by adding the appropriate positive control compound in final concentration of 60 μM in media. The plate was prepared by adding 4 μL negative control to column 23, 4 μL positive control to column 2 and 4 μL cell suspension to all wells in the plate. The plate with cells was then incubated at 37° C. for 2 hours. The assay signal reagent is the Caspase-Glo solution described above, and 8 μL was added to all wells. The plates were then sealed and measured after 30 minutes.

The activity of a test compound was calculated as percent change in apoptosis induction as follows:

$$\begin{matrix}LC = median of the Low Control values \\ = Central Reference in Screener \\ = DMSO \\ = 0 \%\end{matrix}$$

$$\begin{matrix}HC = Median of the High Control values \\ = Scale Reference in Screener \\ = 30 μ M of positive control \\ = 100 \% apoptosis induction\end{matrix}$$

% Effect (AC₅₀)=100−((sample−LC)/(HC−LC))*100

% Control=(sample/HC)*100

% Control min=(sample−LC)/(HC−LC)*100

TABLE
Measured AC₅₀ for Representative Compounds of Formula
(I). Averaged values are reported over all runs
on all batches of a particular compound.

| | MOLP8 Caspase-Glo |
|---|---|
| Compound | AC₅₀ (μM) |
| 1 | 1.22 |
| 2 | 0.054 |
| 3 | >30 |
| 4 | 0.046 |
| 5 | 10.20 |
| 6 | 0.058 |
| 7 | 20.39 |
| 8 | 0.015 |
| 9 | 8.96 |
| 10 | 0.010 |
| 10a | 0.068 |
| 11 | 1.62 |
| 11a | 2.38 |
| 12 | n.d. |
| 12a | 0.029 |
| 13 | 0.022 |
| 14 | 3.46 |
| 15 | 1.40 |
| 16 | 0.013 |
| 17 | 0.015 |
| 18 | 2.43 |
| 19 | 29.5 |
| 20 | 30.0 |
| 21 | 0.082 |
| 22 | 0.022 |
| 23 | 0.027 |
| 24 | 2.53 |
| 25 | 4.13 |
| 26 | 0.235 |
| 27 | 0.117 |

1. A compound of Formula (I): 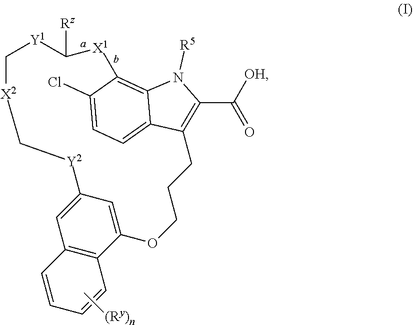
or a tautomer or a stereoisomeric form thereof, wherein:
X¹ is 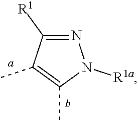
wherein ‘a’ and ‘b’ indicate how variable X¹ is attached to the remainder of the molecule;
R^{y} is halo;
n is 0, 1 or 2;
R^{z} is hydrogen or C₁₋₄alkyl;
X² is 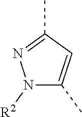
which is attached to the remainder of the molecule in both directions;
R¹ is hydrogen; or C₁₋₆alkyl optionally substituted with one substituent that is Het¹, NR^{6a}R^{6b}, or —OR³;
R² is hydrogen; methyl; or C₂₋₆alkyl optionally substituted with one substituent that is Het¹, NR^{6a}R^{6b}, or —OR³;
R^{1a} is methyl or ethyl;
R³ is hydrogen, C₁₋₄alkyl, or —C₂₋₄alkyl-O—C₁₋₄alkyl;
R₅ is hydrogen; or C₁₋₄alkyl optionally substituted with one substituent that is C₃₋₆cycloalkyl, Het¹, OR⁴, or NR^{6a}R^{6b};
Het¹ is morpholinyl or tetrahydropyranyl;
R⁴ is hydrogen, C₁₋₄alkyl, or —C₂₋₄alkyl-O—C₁₋₄alkyl;
R^{6a} and R^{6b} are each independently hydrogen or C₁₋₄alkyl;
Y¹ is —S—, —O—, —CH₂—;
Y² is —(CH₂)ₘ— or —S—; and
m is 1 or 2;
or a pharmaceutically acceptable salt, or a solvate thereof.

2. The compound according to claim 1, wherein:
R¹ is hydrogen; or C₁₋₆alkyl optionally substituted with one substituent that is Het¹ or —OR³; and
R² is hydrogen; methyl; or C₂₋₆alkyl optionally substituted with one substituent that is Het¹ or —OR³.

3. The compound according to claim 1, wherein R⁵ is C₁₋₄alkyl optionally substituted with one substituent that is C₃₋₆cycloalkyl or Het¹.

4. The compound according to claim 1, wherein
n is 0 or 1;
R^{z} is hydrogen;
R¹ is hydrogen; or C₁₋₆alkyl optionally substituted with one —OR³;
R² is methyl;
R^{1a} is methyl;
R³ is —C₂₋₄alkyl-O—C₁₋₄alkyl;
R⁵ is C₁₋₄alkyl;
Y¹ is —S— and;
m is 1.

5. The compound according to claim 1, wherein R^{z} is hydrogen.

6. The compound according to claim 1, wherein n is 1.

7. The compound according to claim 1, wherein Y¹ is —S—.

8. The compound according to claim 1, wherein R^{y} is fluoro.

9. The compound according to claim 1, wherein:
R¹ is methylene substituted with one —OR³;
R² is methyl; and
R³ is —C₂₋₄alkyl-O—C₁₋₄alkyl.

10. A pharmaceutical composition comprising the compound claim 1 and a pharmaceutically acceptable carrier or diluent.

11. A process for preparing the pharmaceutical composition of claim 10 comprising mixing a pharmaceutically acceptable carrier with a therapeutically effective amount of the compound of claim 1.

12-13. (canceled)

14. The method of claim 15, wherein cancer is prostate cancer, lung cancer, pancreatic cancer, breast cancer, ovarian cancer, cervical cancer, melanoma, B-cell chronic lymphocytic leukemia (CLL), acute myeloid leukemia (AML), or acute lymphoblastic leukemia (ALL).

15. A method of treating or preventing cancer, comprising administering to a subject in need thereof, a therapeutically effective amount of a compound of claim 1 to the subject.
